(19) United States
(12) Patent Application Publication
(10) Pub. No.: US 2014/0121201 A1
(43) Pub. Date: May 1, 2014

(54) REGULATORY NETWORK FOR Th17 SPECIFICATION AND USES THEREOF

(71) Applicants: Dan Littman, New York, NY (US); Maria Ciofani, New York, NY (US); Richard A. Bonneau, JR., New York, NY (US)

(72) Inventors: Dan Littman, New York, NY (US); Maria Ciofani, New York, NY (US); Richard A. Bonneau, JR., New York, NY (US)

(21) Appl. No.: 14/035,533

(22) Filed: Sep. 24, 2013

Related U.S. Application Data
(60) Provisional application No. 61/704,803, filed on Sep. 24, 2012.

(51) Int. Cl. G01N 33/50; A61K 31/55

(52) U.S. Cl. CPC G01N 33/505; A61K 31/55
USPC 514/217.01; 435/29

(57) ABSTRACT

Screening assays and methods of using same for screening to identify modulator agents or compounds that affect Th17 cell specification are described herein. Pharmaceutical compositions comprising agents or compounds that modulate Th17 cell specification are also encompassed. Methods for modulating Th17 cell specification using agents identified using assays described herein in pharmaceutical compositions are also envisioned. Such pharmaceutical compositions are useful for treating inflammatory conditions and autoimmune diseases associated with Th17 cell mediated pathology.

### CROSS REFERENCE TO RELATED APPLICATION

This application claims priority under 35 USC §119(e) from U.S. Provisional Application Ser. No. 61/704,803, filed Sep. 24, 2012, which application is herein specifically incorporated by reference in its entirety.

### GOVERNMENTAL SUPPORT

The research leading to the present invention was supported, at least in part, by National Institutes of Health Grant Nos. RC1 AI087266, RC4 AI092765, PN2 EY016586, IU54CA143907-01, and EY016586-06; and National Science Foundation grant IOS-1126971. Accordingly, the Government has certain rights in the invention.

### FIELD OF THE INVENTION

The present invention relates to methods for screening to identify modulator agents or compounds that affect Th17 cell specification. Also encompassed herein are methods for modulating Th17 cell specification. Pharmaceutical compositions comprising agents or compounds that modulate (inhibit or enhance) Th17 cell specification are also encompassed herein. Autoimmune and inflammatory disorders are exemplary conditions for which such pharmaceutical compositions would confer benefit to patients. The invention further relates to modulator agents or compounds that reduce or inhibit Th17 cell specification and methods of using such agents or compound or pharmaceutical compositions thereof to treat autoimmune and inflammatory disorders.

### BACKGROUND OF THE INVENTION

The citation of references herein shall not be construed as an admission that such is prior art to the present invention.

The vertebrate immune system, composed of numerous phenotypically well-defined cell types, is ideally suited for studying the combinatorial action of transcription factors (TFs) and epigenetic regulators whose target gene products confer unique cellular functions. TFs that are selectively expressed in subsets of myeloid and lymphoid lineage have been designated “master regulators” if they are both essential and sufficient to induce defined cell fates. It is becoming clear, however, that networks of multiple TFs are required to achieve the full differentiation programs (Mattick et al., 2010; Novershtern et al., 2011). How such factors cooperate to determine specific programs remains poorly understood.

CD4-expressing T lymphocytes are among the best-characterized immune system cells (Zhu et al., 2010). They develop in the thymus and acquire the potential to become T-helper cells that guide B lymphocytes to produce distinct classes of antibody and carry out multiple other effector functions; or they up-regulate the TF Foxp3 and become anti-inflammatory regulatory T cells (Treg). T-helper cells differentiate further in the periphery following induction or activation of TFs in response to signals from the T cell antigen receptor (TCR), cytokines, and other ligands in the microenvironment. T-helper effector subsets include Th1 cells, which produce interferon-γ and control infections with intracellular microbes, Th2 cells, which secrete IL-4, IL-5, and IL-13 and are required for clearance of helminths, and Th17 cells, producers of IL-17A, IL-17F, and IL-22 that protect mucosa from bacterial and fungal infection (Korn et al., 2009). In addition, follicular helper T cells (T_{FH}) provide B cells with signals for immunoglobulin class switching and affinity maturation (Crotty, 2011). CD4⁺ T cell subsets exhibit plasticity, but are considered distinct lineages based on expression of TFs with properties of “master regulators”. Th1 cells are defined by their expression of T-bet (Tbx21), Th2 cells by GATA3, Th17 cells by RORγt, and T_{FH} cells by Bcl6. Effector T cells expressing distinct subset-specific cytokines are common in vivo, although cells with combinations of such cytokines are often observed. Differentiation of naïve CD4⁺ T cells into Th1, Th2, Th17, or Treg cells can be mimicked in vitro by TCR stimulation and combinations of defined cytokines. Genome-wide histone modifications, chromatin accessibility, and occupancy by lineage-specifying TFs have been studied in such models (Durant et al., 2010; Kwon et al., 2009; Wei et al., 2009).

Th17 cells have critical functions in many autoimmune diseases and in cancer (Korn et al., 2009). The orphan nuclear receptor RORγt is required for the differentiation of Th17 cells and for inflammatory diseases in mice. Its forced expression in mouse and human T cells induces transcripts present in Th17 cells, including those coding for the key cytokines, for the IL-23 receptor, and for the chemokine receptor CCR6 (Ivanov et al., 2006; Manel et al., 2008). However, RORγt is not sufficient to specify the full Th17 program, and other TFs, including STAT3, IRF4, BATF, and IκB ζ, are required for induction of RORγt and IL-17A in vivo and upon polarization in vitro with IL-6, TGF-β, with or without IL-1β and IL-23 (Brustle et al., 2007; Okamoto et al., 2010; Schraml et al., 2009; Yang et al., 2007). Multiple other TFs are also involved in Th17 cell differentiation, including c-Maf, Runx1, and Ahr (Bauquet et al., 2009; Veldhoen et al., 2008; Zhang et al., 2008). RORα, which is closely related to RORγt, can also contribute to IL-17 expression in the absence of RORγt (Yang et al., 2008).

### SUMMARY OF THE INVENTION

Investigation of TF functions in Th17 cell differentiation has been limited to how single factors affect expression of a limited number of targets (e.g. IL-17A). However, the Th17 differentiation program extends beyond functions of individual cytokines, as highlighted by studies showing Th17-mediated pathogenesis in the absence of IL-17A and IL-17F (Codarri et al., 2011; Leppkes et al., 2009). We therefore wished to examine how multiple TFs regulate each other and their targets in order to model a transcriptional network and identify novel critical factors in Th17 cell differentiation. New regulatory interactions can be found using genome-wide methods to learn networks from time series and genetic and environmental perturbations (Bonneau et al., 2007; Faith et al., 2007; Greenfield et al., 2010). Integrating multiple data types and analytical methods for network inference using meta-analyses (averaging over several data types and computational approaches) can leverage the complementary weaknesses and strengths of each component to produce higher accuracy networks (Marbach et al., 2012).

As described herein, we have applied an integrative approach, with meta-analysis of genome occupancy of multiple TFs, RNA-seq of TF-deficient T cells and immune cell transcriptome data, to build a network model for Th17 cells. We find in Th17 cell differentiation early cooperative binding of BATF and IRF4 that governs chromatin accessibility and subsequent recruitment of RORγt to regulate a select set of Th17-relevant genes. We used the network model to identify additional candidate genes that were in turn incorporated through an iterative process, and validated several genes critical in Th17 cell differentiation, including TFs that influence the expression of >2,000 genes. We found that the AP-1 family member Fos12 (confidently predicted to be a core Th17 factor) has a key role in a mouse model of autoimmune disease, limiting plasticity of T helper cell differentiation. In addition, loci implicated in genome-wide association studies (GWAS) to have roles in autoimmune disease were enriched in the Th17 network. This analysis can therefore identify candidate genes that serve as cogs in functional specialization of Th17 cells and that have potential to be valuable for new therapeutic approaches.

In accordance with the present findings, a method for screening to identify a modulator of a Th17 regulatory network (TRN) protein is presented, the method comprising: contacting a population of naïve CD4⁺ T cells polarized under Th17 conditions with at least one candidate agent and assessing at least one transcriptional readout of TRN protein activity in the presence or absence of the at least one candidate agent, wherein a change in level of the at least one transcriptional readout indicates that the least one candidate agent is a TRN protein modulator.

In a particular embodiment thereof, wherein the change detected in the presence of the candidate modulator agent is a reduction in the at least one transcriptional readout, the candidate modulator agent is identified as an inhibitor of the TRN protein activity.

In another particular embodiment thereof, wherein the change detected in the presence of the candidate modulator agent is an increase in the at least one transcriptional readout, the candidate modulator agent is identified as an enhancer of the TRN protein activity.

In a further embodiment, the at least one transcriptional readout involves detecting expression of at least one of a transcription factor-dependent loci selected from the group consisting of Il17a, Il17f, Il22, Illr1, Il23r, Il10, Il24, Il9, Ccl20, Il4, Ifng, Gata3, Foxp3, and Tbx21. In a more particular embodiment, the at least one transcription factor-dependent loci is selected from the group consisting of Il17a and Il17f.

In a further embodiment, the at least one transcriptional readout involves detecting expression of at least one of a lineage-specializing gene selected from the group consisting of Rorc, Gata3, Foxp3, Tbx21, 114, and Ifng. In a more particular embodiment, the at least one lineage-specializing gene is Rorc.

In a still further embodiment, the at least one transcriptional readout is detecting expression of a Th17 cell cytokine. In a more particular embodiment, the Th17 cell cytokine is IL17A, IL17F, IL22, or IL21.

In yet another embodiment, the at least one transcriptional readout is an exogenous marker whose expression is regulated by the TRN protein activity.

In a further aspect of the method, the candidate modulator agent is a small organic molecule, a protein, a peptide, a nucleic acid, a carbohydrate, or an antibody.

As detailed herein, the TRN protein is selected from those proteins identified as playing a role in a Th17 cell regulatory network. In a particular embodiment, the TRN protein is selected from those listed in Table S4. In a more particular embodiment, the TRN protein is selected from those listed in Table 1. In a still more particular embodiment, the TRN protein is selected from those listed in Table 2. In an even more particular embodiment, the TRN protein is selected from Fos12 [National Center for Biotechnology Information (NCBI) Gene ID Nos. 14284 and 2355, mouse and human, respectively], Etv6 NCBI Gene ID Nos. 14011 and 2120, mouse and human, respectively), Nfatc2 (NCBI Gene ID Nos. 18019 and 4773, mouse and human, respectively), Crem (NCBI Gene ID Nos. 12916 and 1390, mouse and human, respectively), Satb1 (NCBI Gene ID Nos. 20230 and 6304, mouse and human, respectively), Bcl11b (NCBI Gene ID Nos. 58208 and 64919, mouse and human, respectively), Jmjd3 (NCBI Gene ID Nos. 216850 and 23135, mouse and human, respectively), Ncoa2 (NCBI Gene ID Nos. 17978 and 10499, mouse and human, respectively), Skil (NCBI Gene ID Nos. 20482 and 6498, mouse and human, respectively), and/or Trib3 (NCBI Gene ID Nos. 228775 and 57761, mouse and human, respectively), which proteins are implicated in Th17 specification for the first time herein. It is to be understood that methods TRN proteins described herein also include Smad3 and Hif1a. Even more particularly, the TRN protein is selected from Etv6, Nfatc2, Bcl11b, Crem, Satb1, or Kdm6b (Jmjd3). In even more particular embodiments, the TRN protein is Fos12 or JMJD3. Exemplary nucleic and amino acid sequences for mouse Fos12 (GenBank: AK089371.1) are designated herein as SEQ ID NOs: 1 and 2, respectively. Exemplary nucleic and amino acid sequences for human Fos12 (GenBank: NM_{—}005253) are designated herein as SEQ ID NOs: 3 and 4, respectively. In further embodiments, the TRN protein is Fos12 and the method indicates that a modulator thereof is an inhibitor of Fos12 activity. Accordingly, the method encompasses identification of Fos12 inhibitors which can be used, for example, to inhibit Th17 mediated immune responses and thereby, reduce inflammatory responses to which Th17 cells contribute. In still further embodiments, the TRN protein is JMJD3 and the method indicates that a modulator thereof is an inhibitor of JMJD3 activity. Accordingly, the method encompasses identification of JMJD3 inhibitors which can be used, for example, to inhibit Th17 mediated immune responses and thereby, reduce inflammatory responses to which Th17 cells contribute.

The methods may further comprise a determination of the mechanism whereby the modulator interacts with the TRN to promote or inhibit TRN activity. Such assays may be performed as described herein to determine if the modulator (e.g., an inhibitor) binds directly to the TRN and if so, to what domain of the TRN.

In another aspect, a method for inhibiting Th17 cell specification is presented, the method comprising contacting CD4⁺ T cells polarized under Th17 conditions with at least one inhibitor of the TRN protein activity identified using methods described herein, or expressing a nucleic acid sequence encoding same in the CD4⁺ T cells polarized under Th17 conditions, wherein the contacting or the expressing inhibits a Th17 regulatory network protein activity.

In a particular embodiment, the CD4⁺ T cells polarized under Th17 conditions are in a mammalian subject. In a more particular embodiment thereof, the mammalian subject is afflicted with an inflammatory condition or autoimmune disease linked to Th17 cell-mediated pathology. In an even more particular embodiment, the inflammatory condition or autoimmune disease is Crohn's disease, ulcerative colitis, multiple sclerosis, rheumatoid arthritis, or psoriasis. Even more particularly, the inflammatory condition or autoimmune disease is multiple sclerosis.

In an aspect of the above method, the mammalian subject is a human.

In yet another aspect, a method for screening to identify a modulator of Th17 cell specification is presented, the method comprising contacting a population of naïve CD4⁺ T cells polarized under Th17 conditions with at least one candidate agent and assessing activity of at least one Th17 regulatory network (TRN) protein in the naïve CD4⁺ T cells in the presence or absence of the at least one candidate agent, wherein a change in activity level of the at least one TRN protein identifies the at least one candidate agent as a TRN protein activity modulator and therefore, a modulator of Th17 cell specification. At least one TRN protein may refer to one, two, three, four, five, six, seven, eight, nine, ten, fifteen, twenty, twenty-five, thirty, thirty-five, forty, forty-five, or fifty of the TRN proteins described herein, including whole numbers between the aforementioned numbers of TRN proteins.

In a particular embodiment, the at least one TRN protein is selected from those listed in Table S4. In a more particular embodiment, the at least one TRN protein is selected from those listed in Table 1. In a still more particular embodiment, the at least one TRN protein is selected from those listed in Table 2. In an even more particular embodiment, the at least one TRN protein is selected from Fos12, Etv6, Nfatc2, Crem, Satb1, Bcl11b, Jmjd3, Ncoa2, Skil, and/or Trib3, which proteins are implicated in Th17 specification for the first time herein. It is to be understood that methods TRN proteins described herein may also include Smad3 and Hif1a. Even more particularly, the TRN protein is selected from at least one of Etv6, Nfatc2, Bcl11b, Crem, Satb1, or Kdm6b (Jmjd3). In even more particular embodiments, the at least one TRN protein comprises Fos12 or JMJD3. In further embodiments, the at least one TRN protein comprises Fos12 and the method indicates that a modulator thereof is an inhibitor of Fos12 activity. Accordingly, the method encompasses identification of Fos12 inhibitors which can be used, for example, to inhibit Th17 cell specification and thus, immune responses and inflammatory responses to which Th17 cells contribute. In still further embodiments, the at least one TRN protein comprises JMJD3 and the method indicates that a modulator thereof is an inhibitor of JMJD3 activity. Accordingly, the method encompasses identification of JMJD3 inhibitors which can be used, for example, to inhibit Th17 cell specification and thus, immune responses and inflammatory responses to which Th17 cells contribute.

In a particular embodiment thereof, the activity of the at least one TRN protein is measured by determining levels of at least one transcriptional readout as described herein. Accordingly, in one embodiment, the presence of the candidate modulator agent leads to a reduction in the activity of the at least one TRN protein as reflected by a reduced level of at least one transcriptional readout, thereby identifying the candidate modulator agent as an inhibitor of the at least one TRN protein activity. In another particular embodiment, the presence of the candidate modulator agent leads to an increase in the activity of the at least one TRN protein as reflected by an increased level of at least one transcriptional readout, thereby identifying the candidate modulator agent as an enhancer of the at least one TRN protein activity.

Also encompassed herein is a method for treating a subject afflicted with an inflammatory condition or autoimmune disease associated with Th17 cell mediated pathology, the method comprising administering the at least one inhibitor of the TRN protein activity identified using methods described herein to the subject, wherein the at least one inhibitor of the TRN protein activity reduces Th17 cell activity in the subject and thereby treats the subject.

Also envisioned herein is a method for treating a subject afflicted with an inflammatory condition or autoimmune disease associated with Th17 cell mediated pathology, the method comprising administering GSK-J1 to the subject, wherein the GSK-J1 reduces Th17 cell activity in the subject and thereby treats the subject.

Other objects and advantages will become apparent to those skilled in the art from a review of the ensuing detailed description, which proceeds with reference to the following illustrative drawings, and the attendant claims.

### BRIEF DESCRIPTION OF THE DRAWINGS

FIG. 1. Genome-wide co-occupancy of Th17 lineage TFs.

A. ChIP-seq binding tracks for core TFs, p300, and CTCF at selected Th17 loci in Th0 and Th17 cells at 48 h (visualized with IGV, Broad Institute).
B. A clustered heat map of pCRM regions (rows) based on TF ChIP signals and the associated gene expression fold changes (FC) in Th17 vs. Th0 cells. A schematic illustration of the clustering approach is shown (top panel).
C. Numbers of pCRMs with >500 occurrences in Th17 cells (bottom) with associated distributions of TF ChIP p-values (top). Boxplots (top) show median (line), 25^{th} to 75^{th} percentile (box)±1.5 interquartile range. * denotes that we observed significantly more pCRMs than expected by chance based on 10000 simulations (P value<0.001).
D. Luciferase reporter assay of enhancer activity for selected pCRM DNA regions in CD4 T cells cultured under Th2 and Th17 conditions. Y-axis: luciferase activity relative to that of pGL4-minP with a minimal promoter. X-axis: TF occupancy order of tested pCRMs (1 to 5 TFs); 1117a-5 and 1117a-19 serve as positive and negative controls, respectively. Error bars=SD of two experiments.

FIG. 2. Cooperative occupancy by BATF and IRF4.

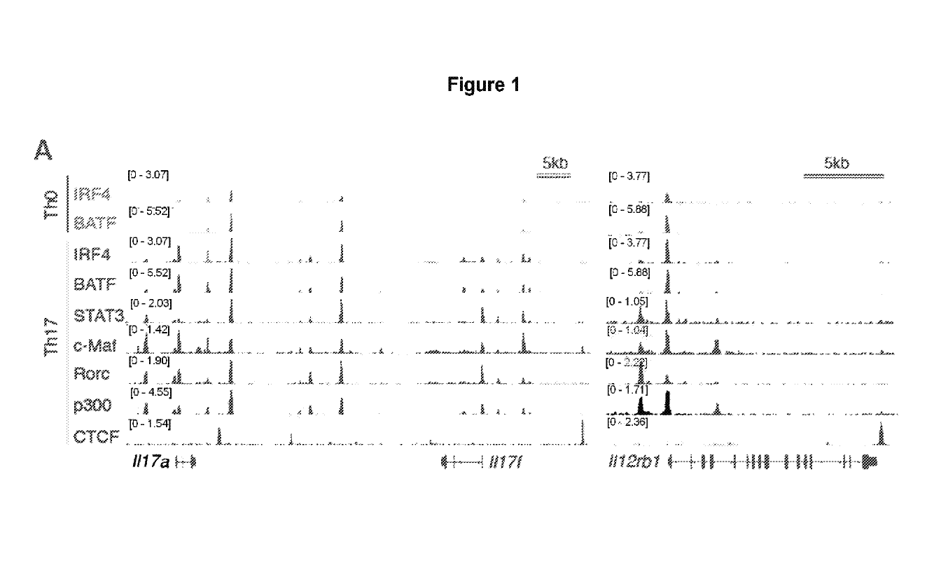

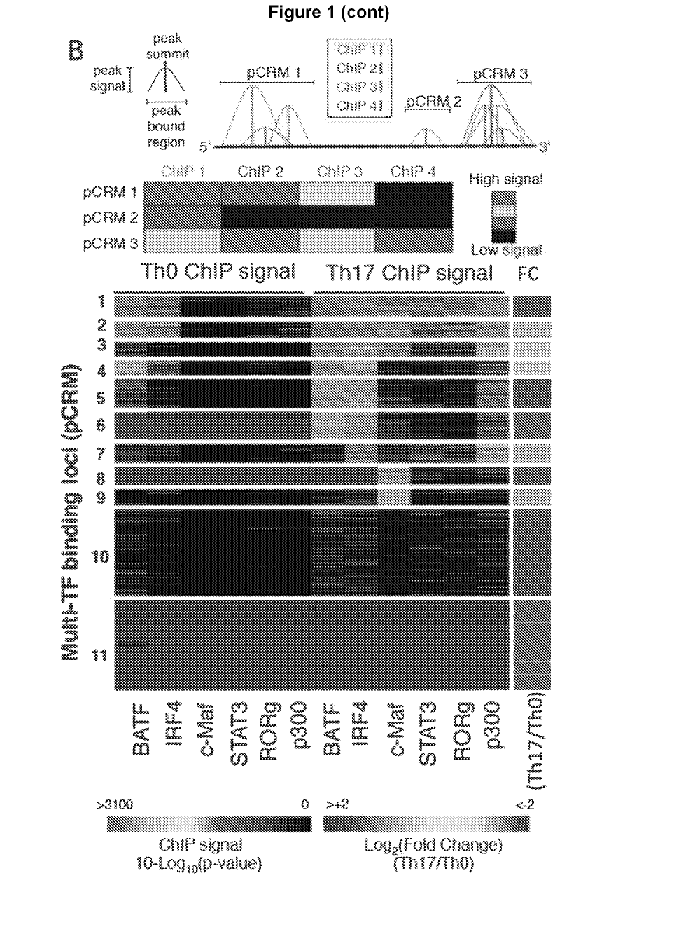

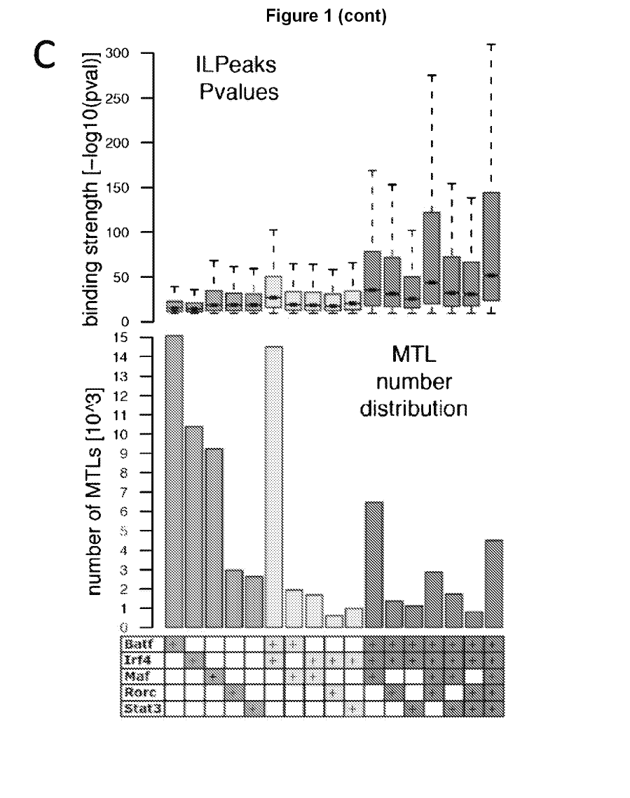

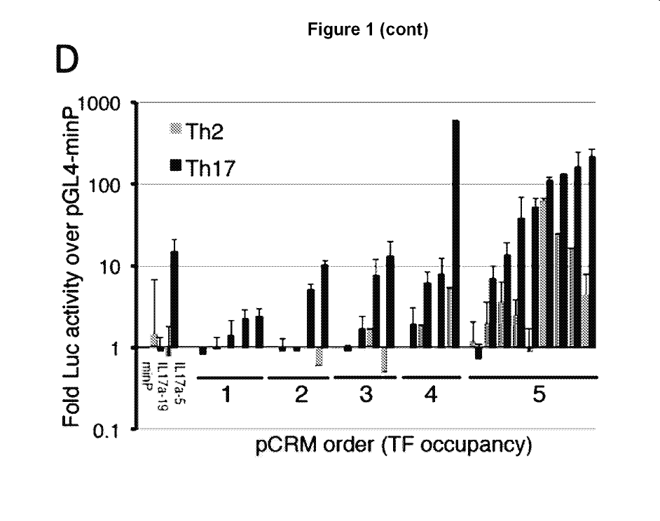

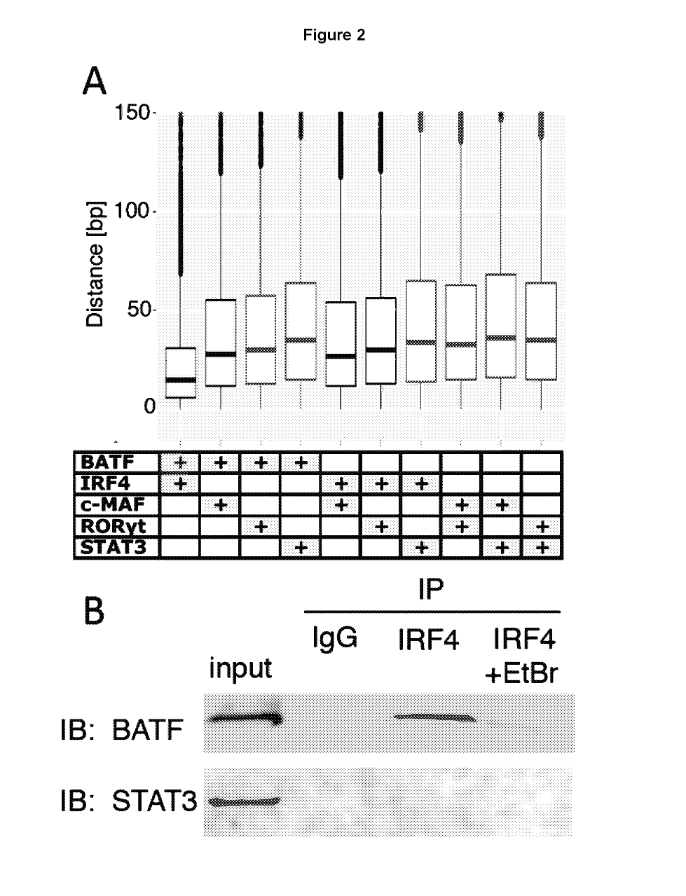

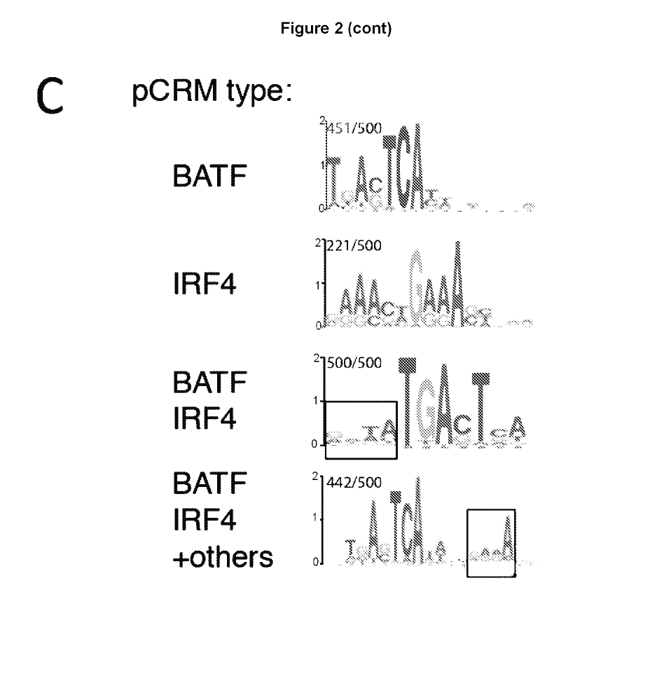

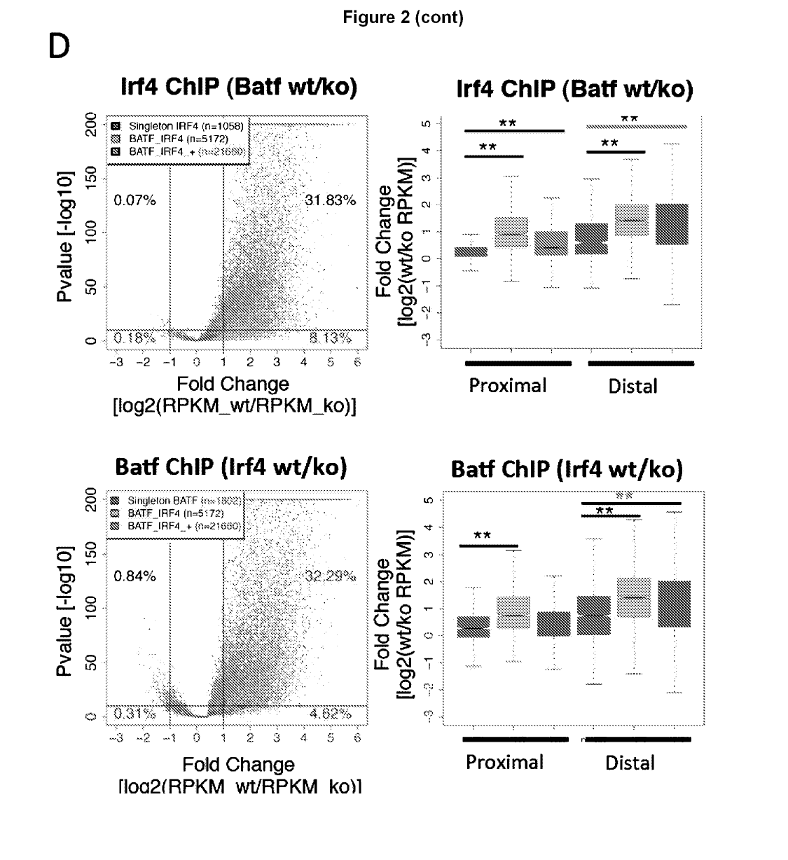

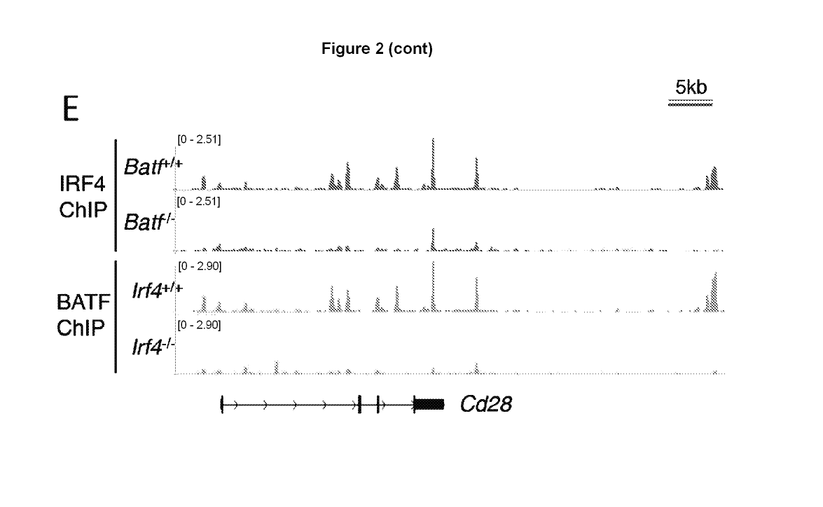

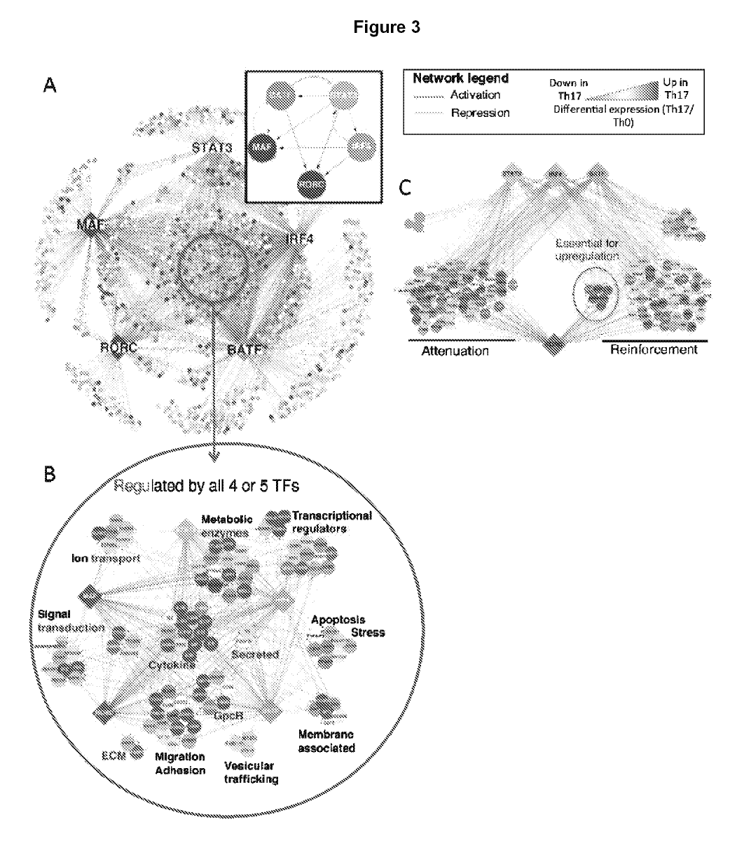

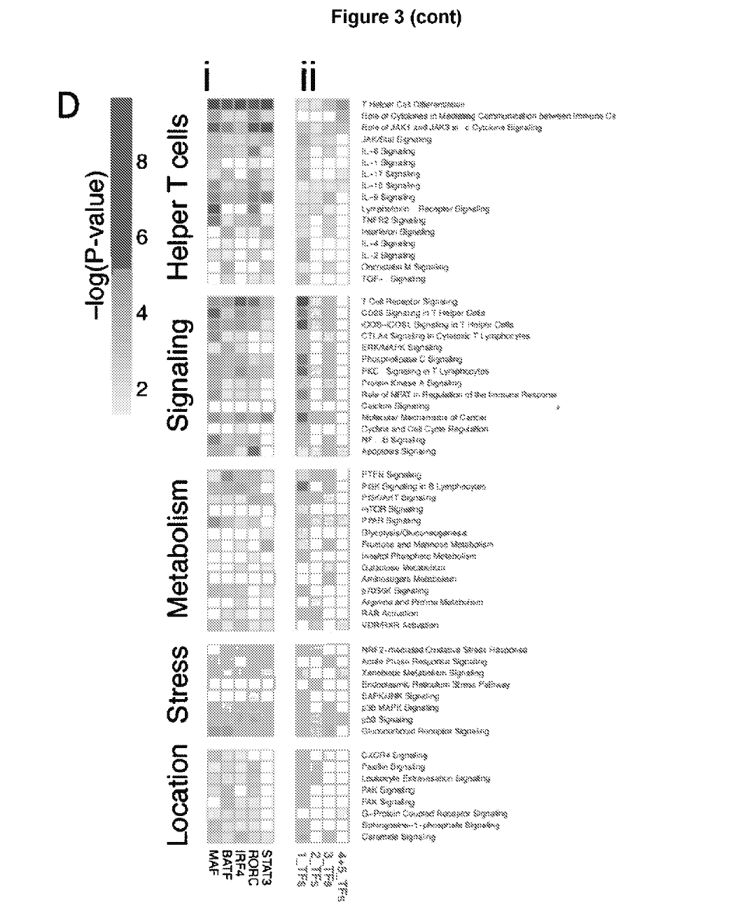

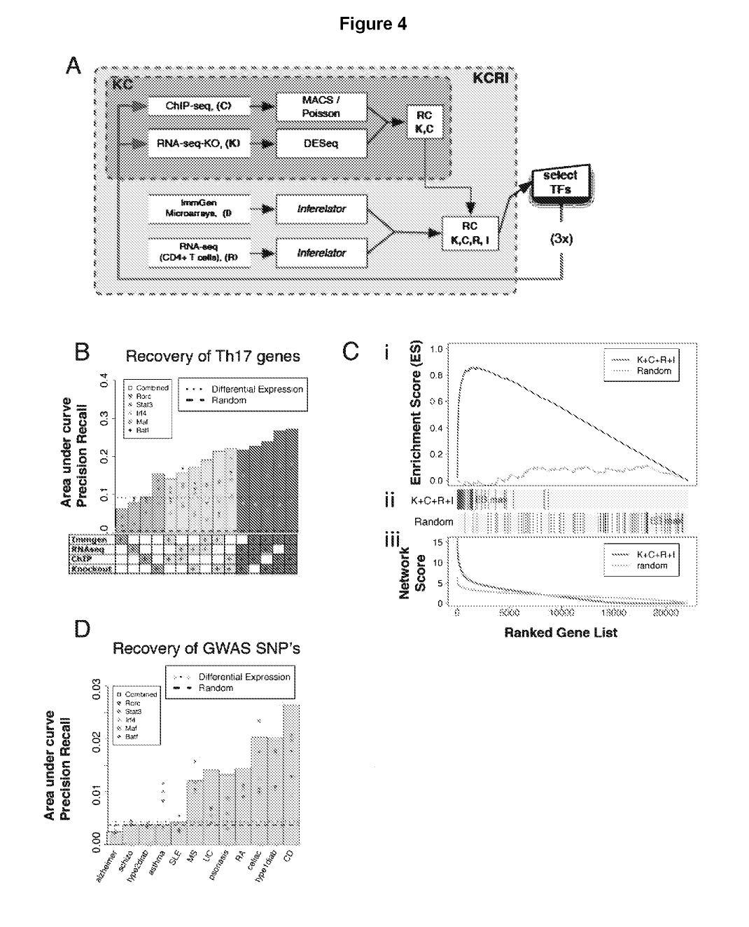

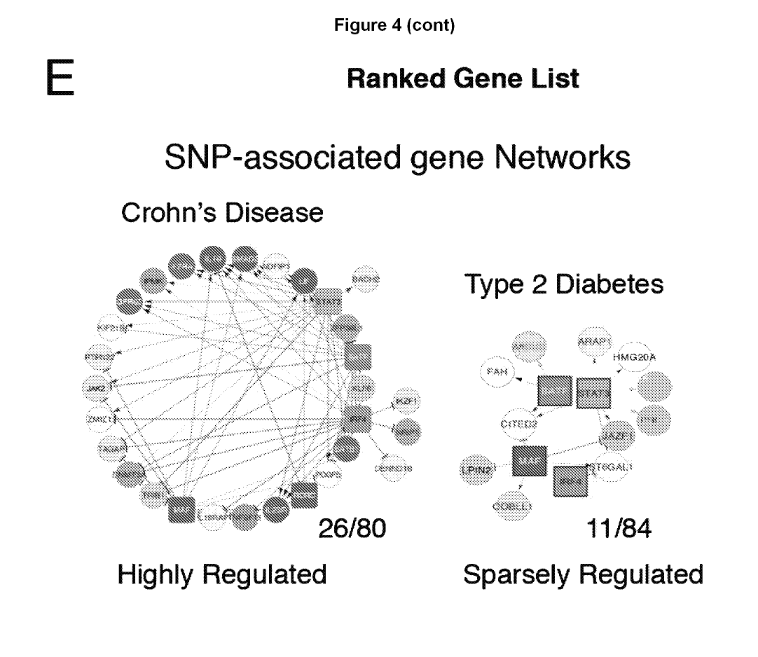

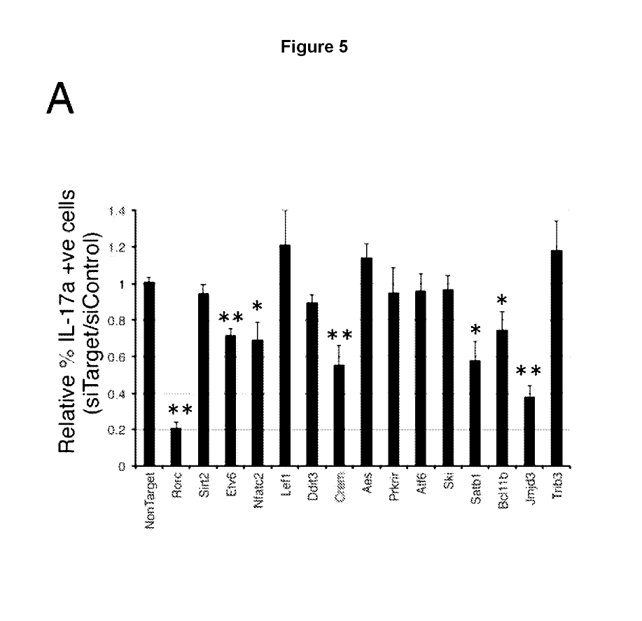

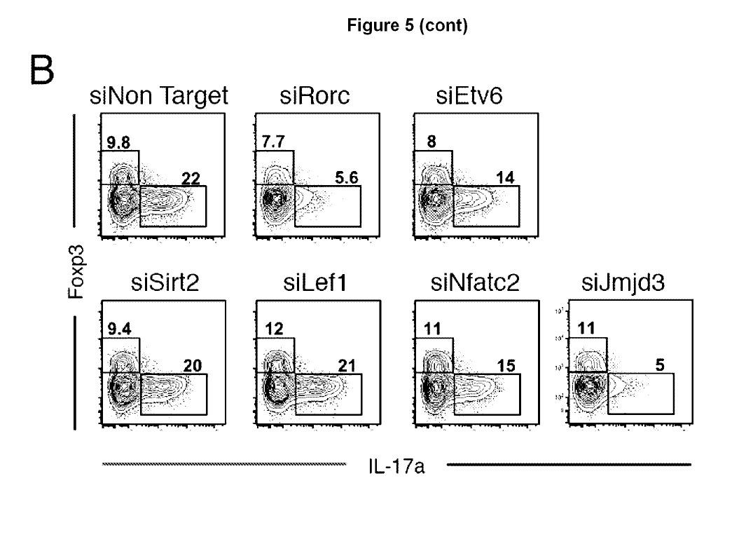

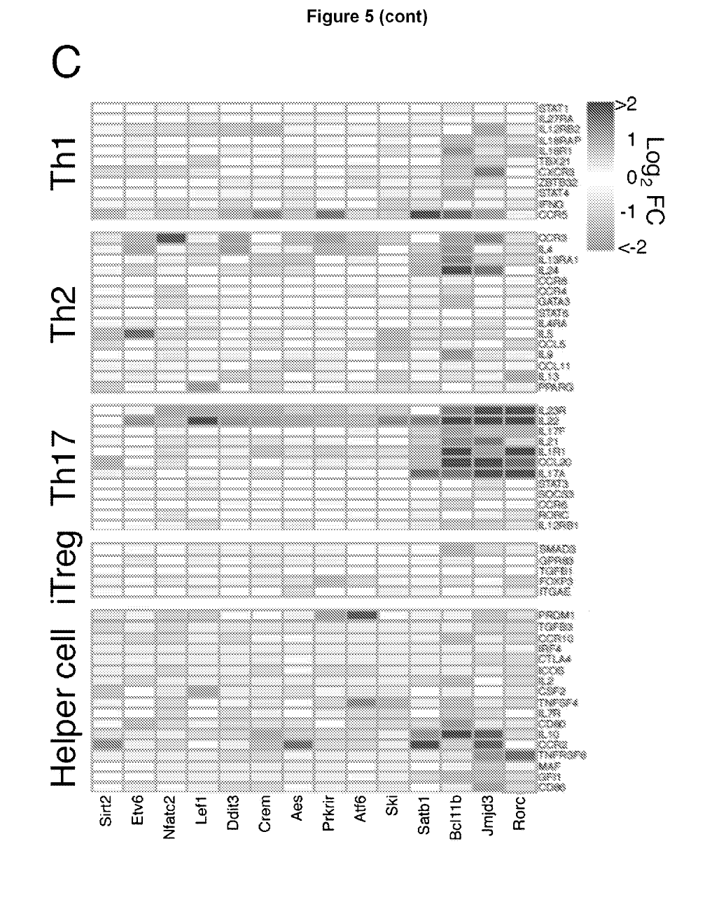

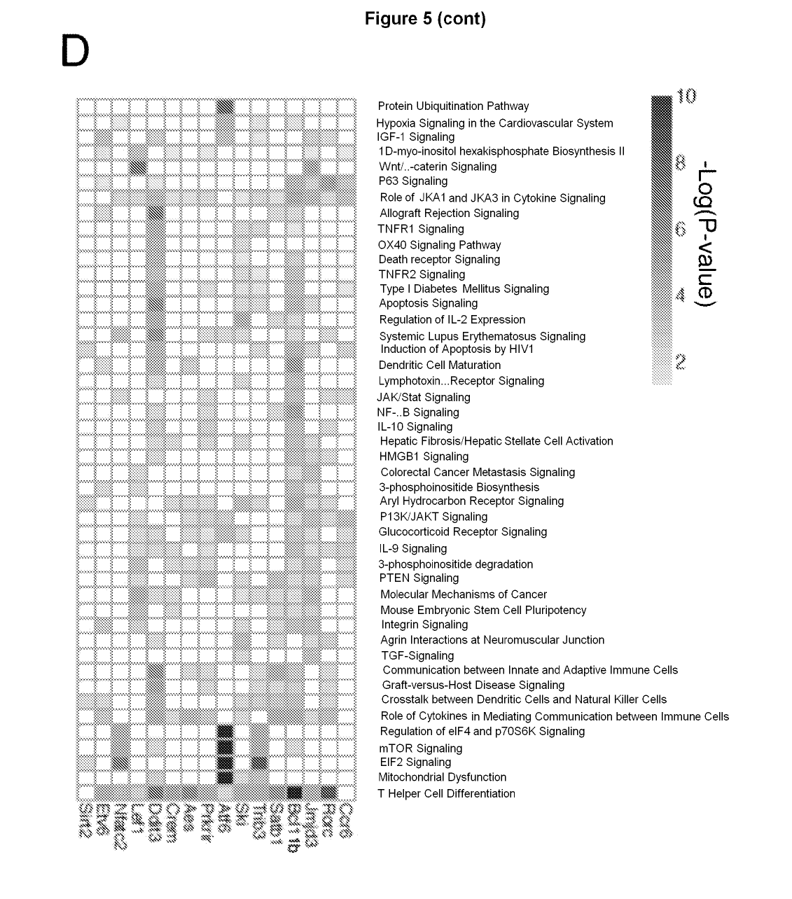

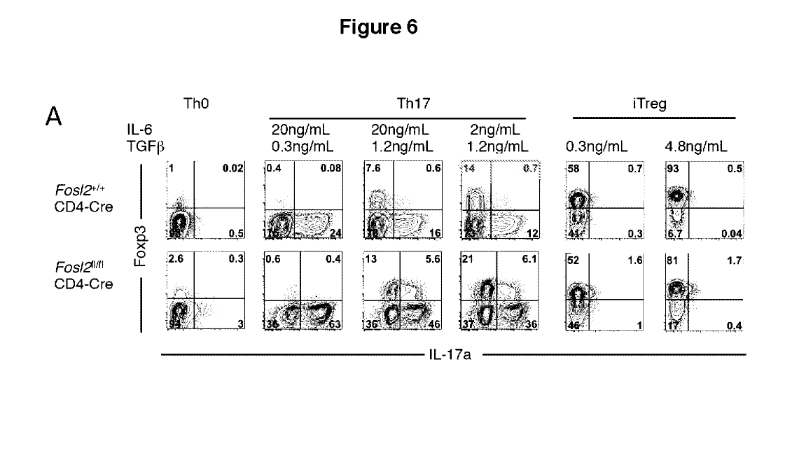

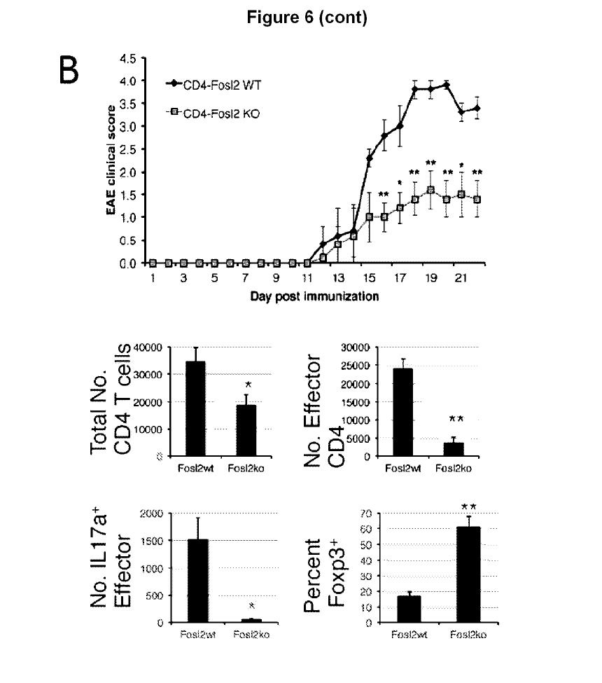

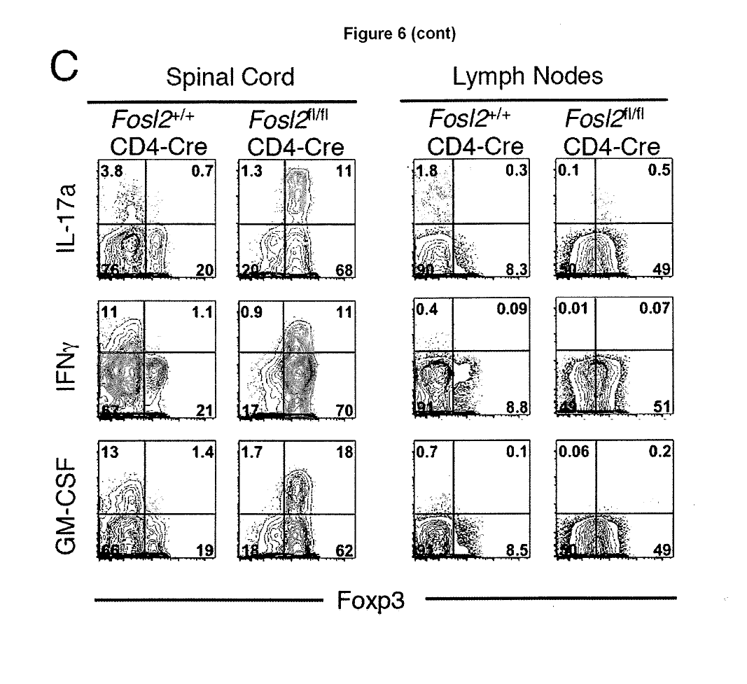

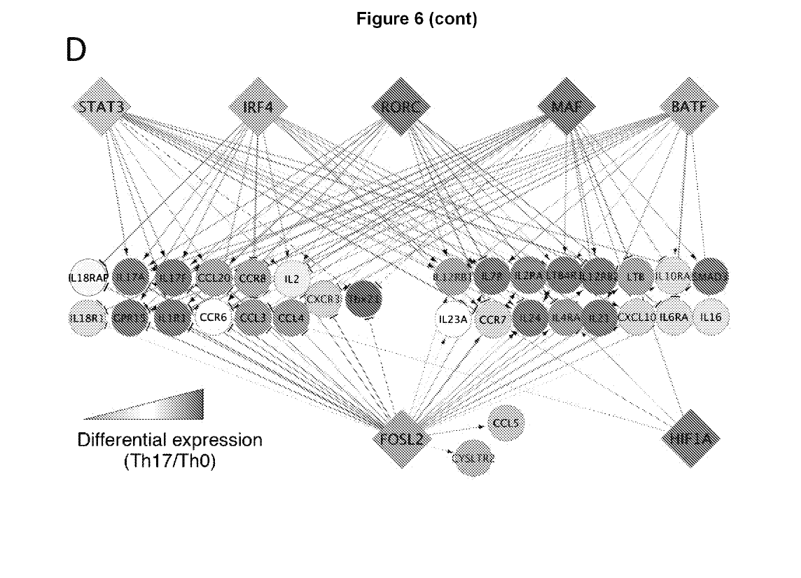

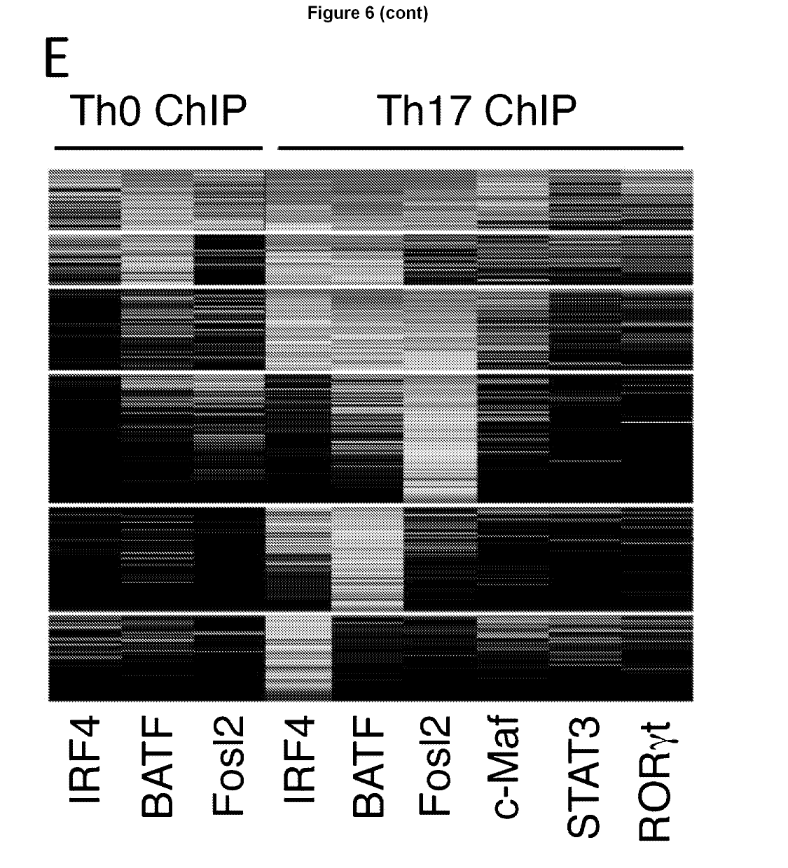

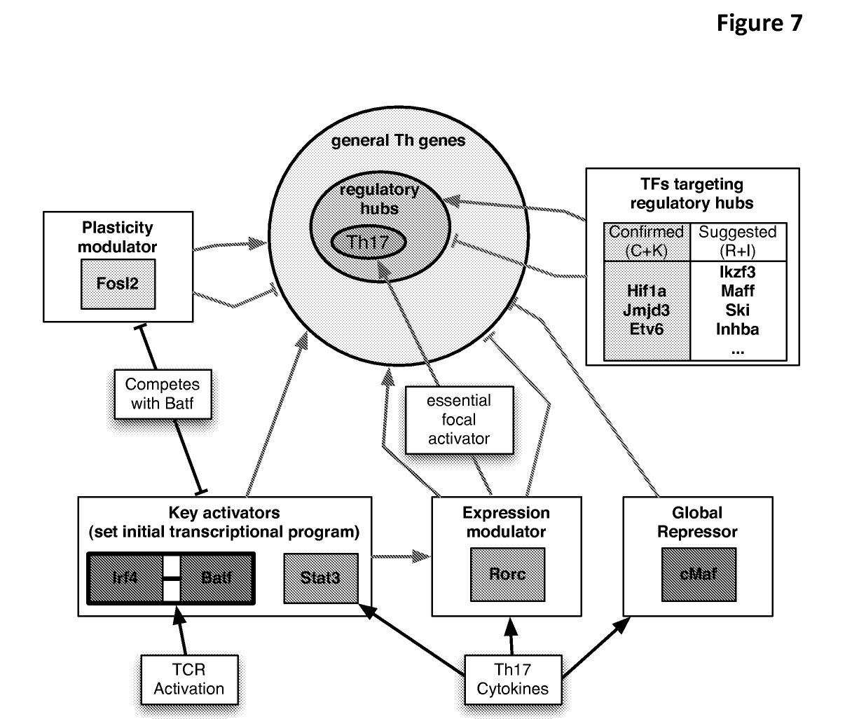

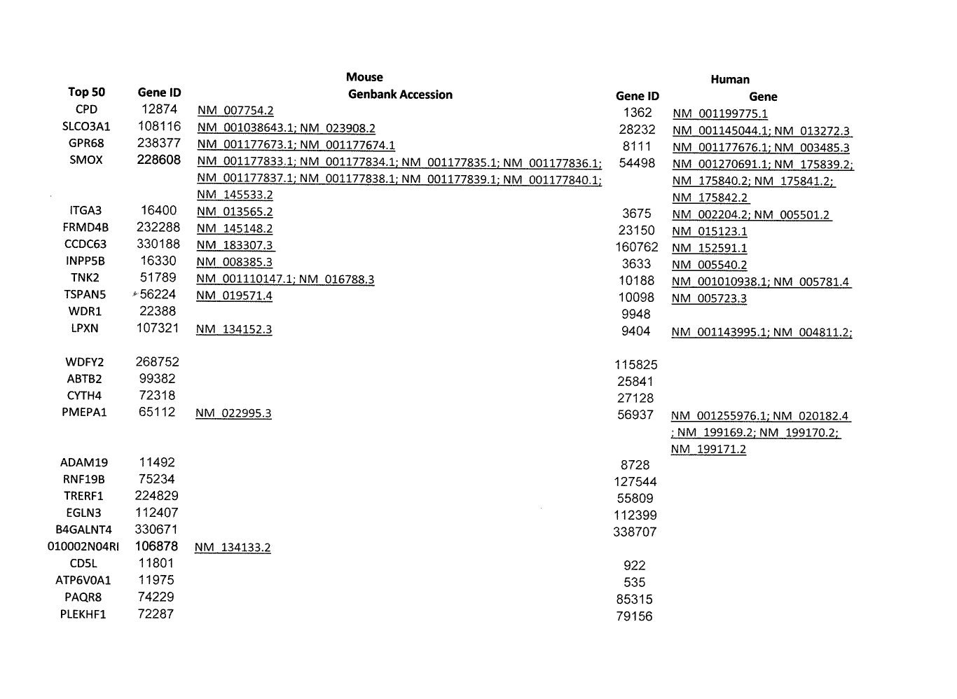

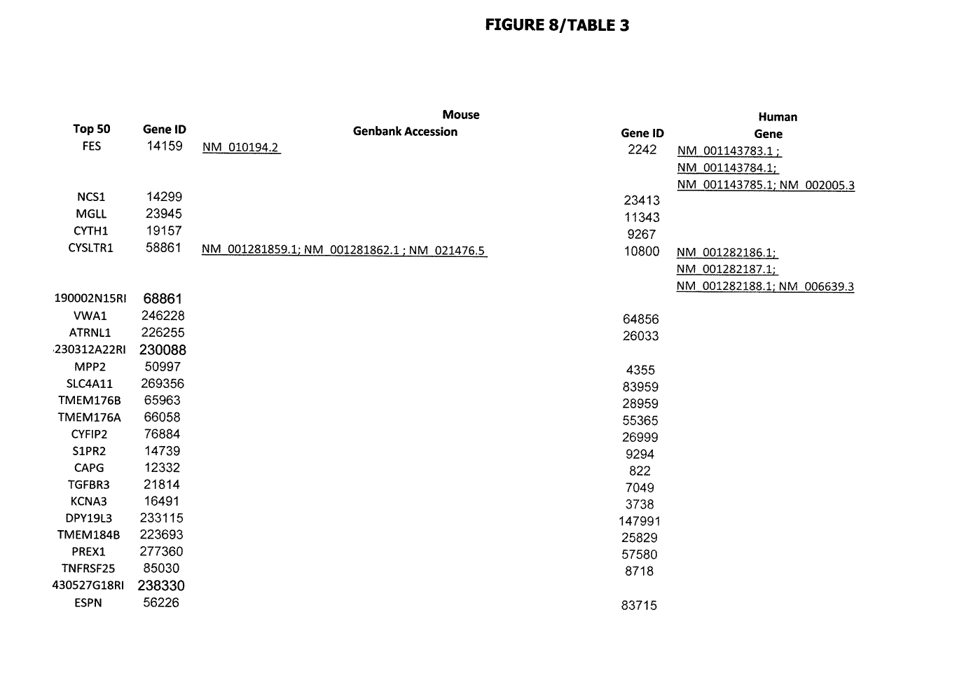

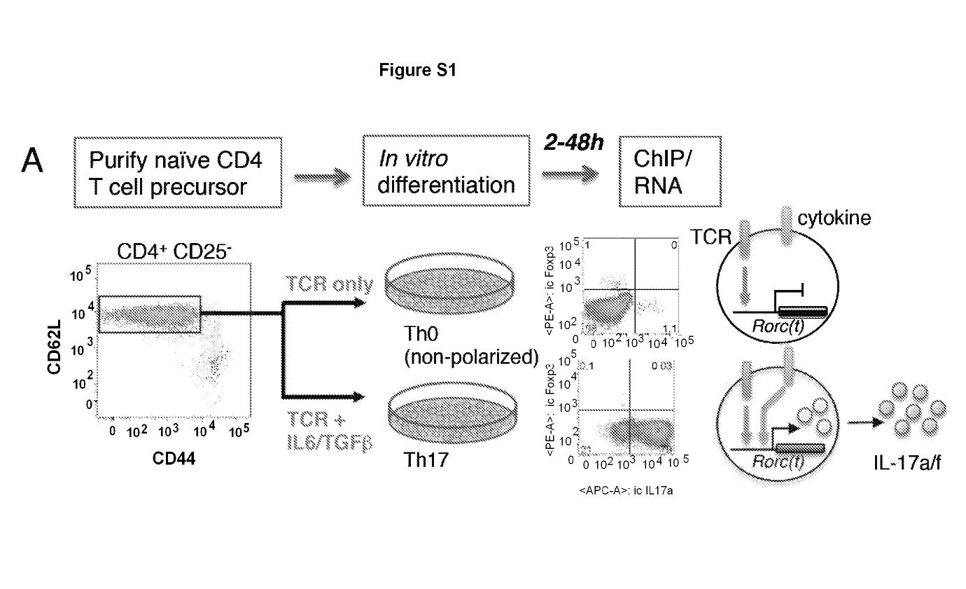

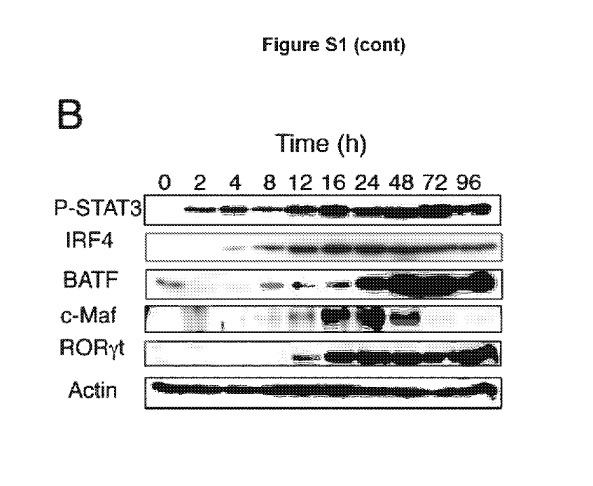

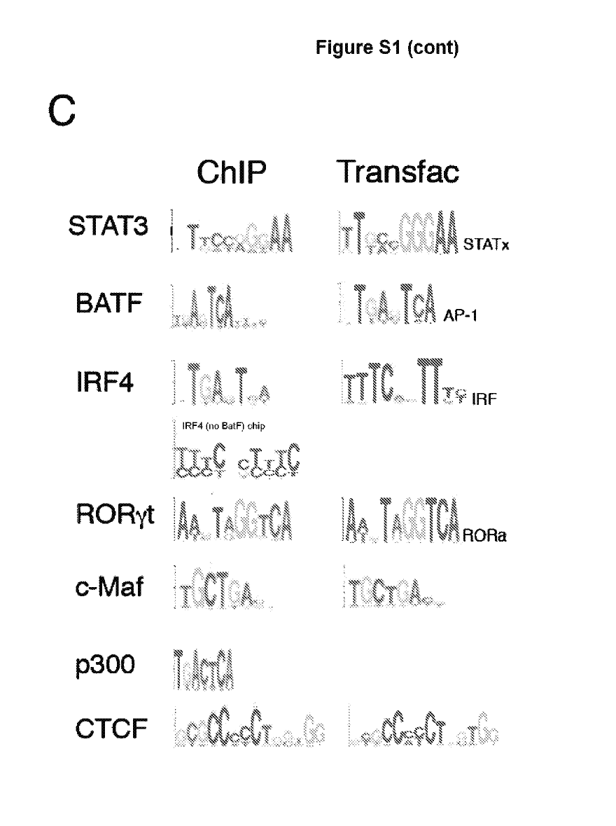

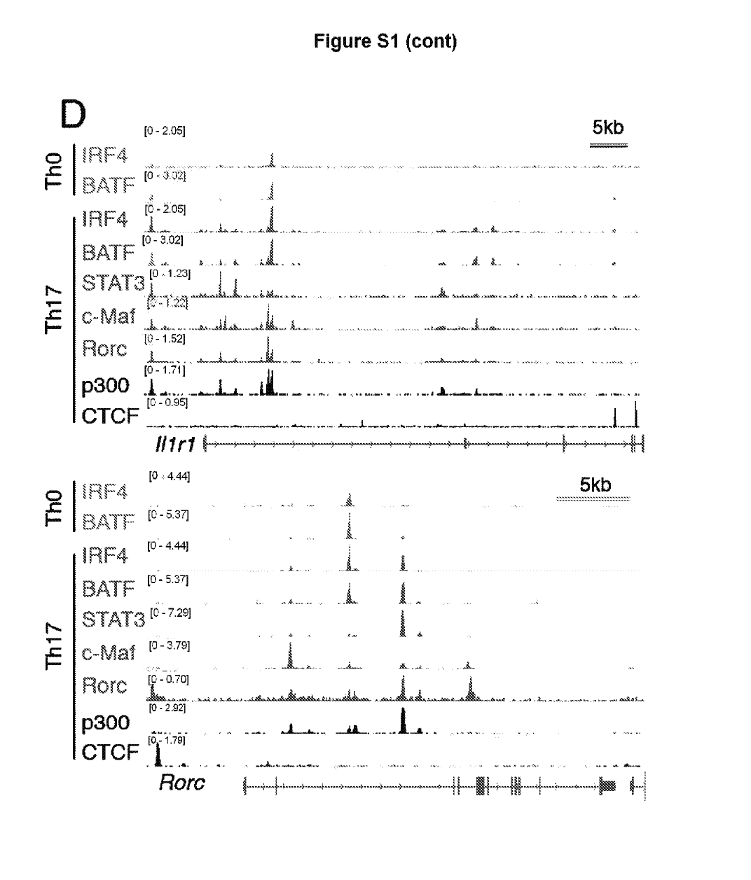

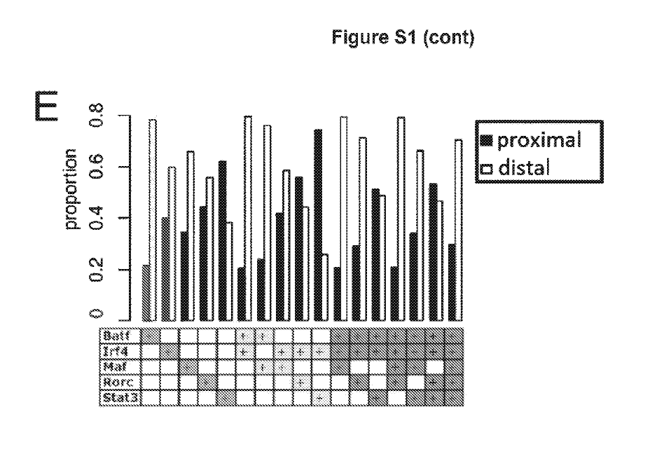

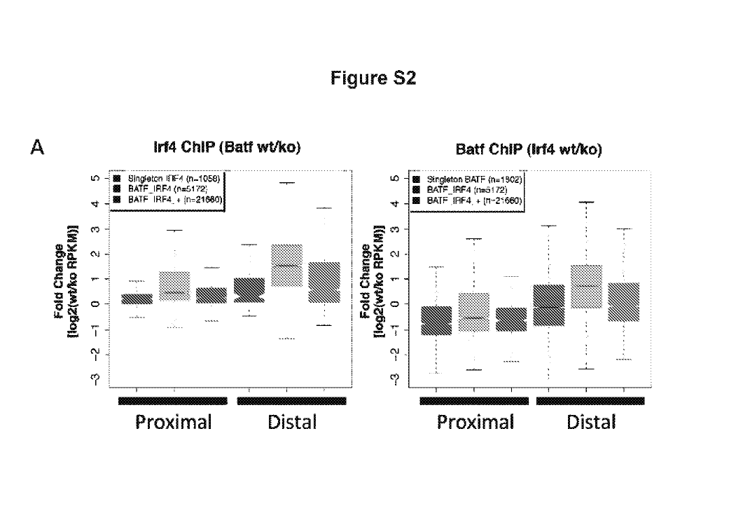

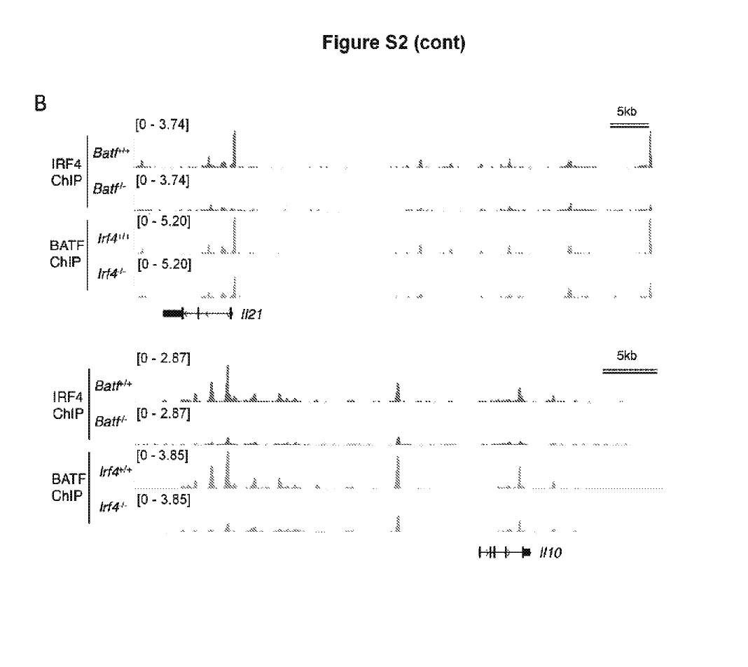

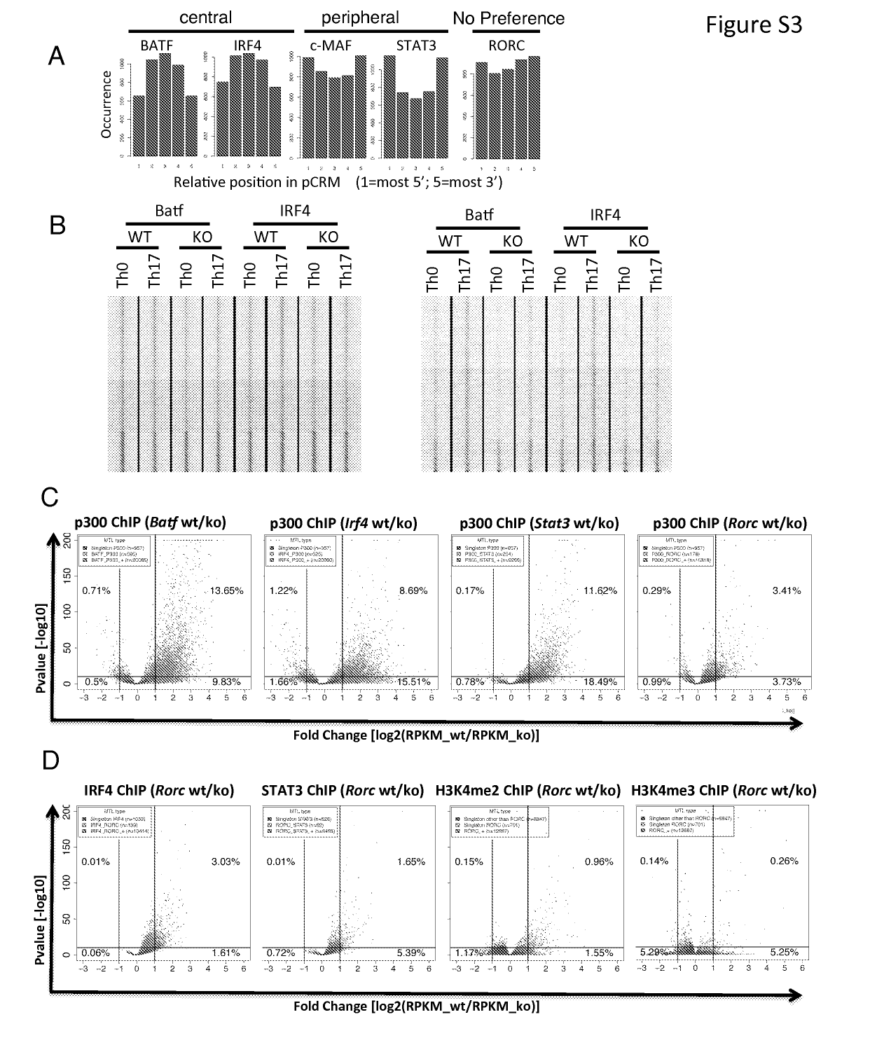

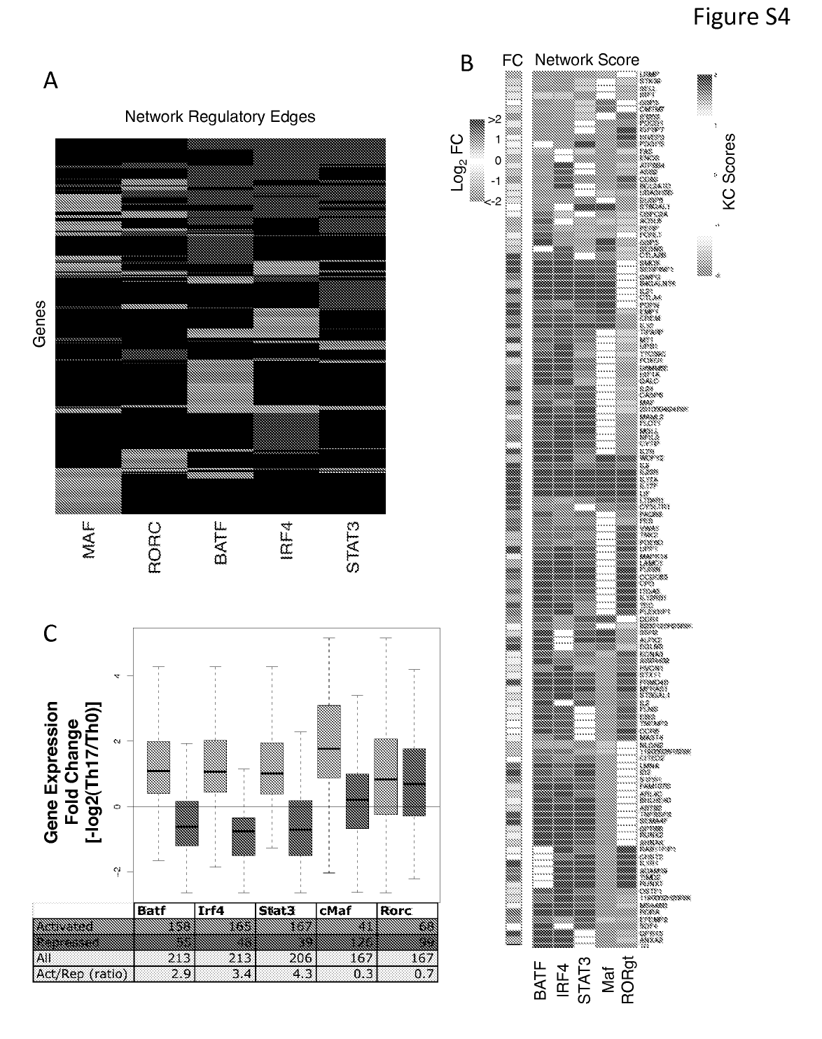

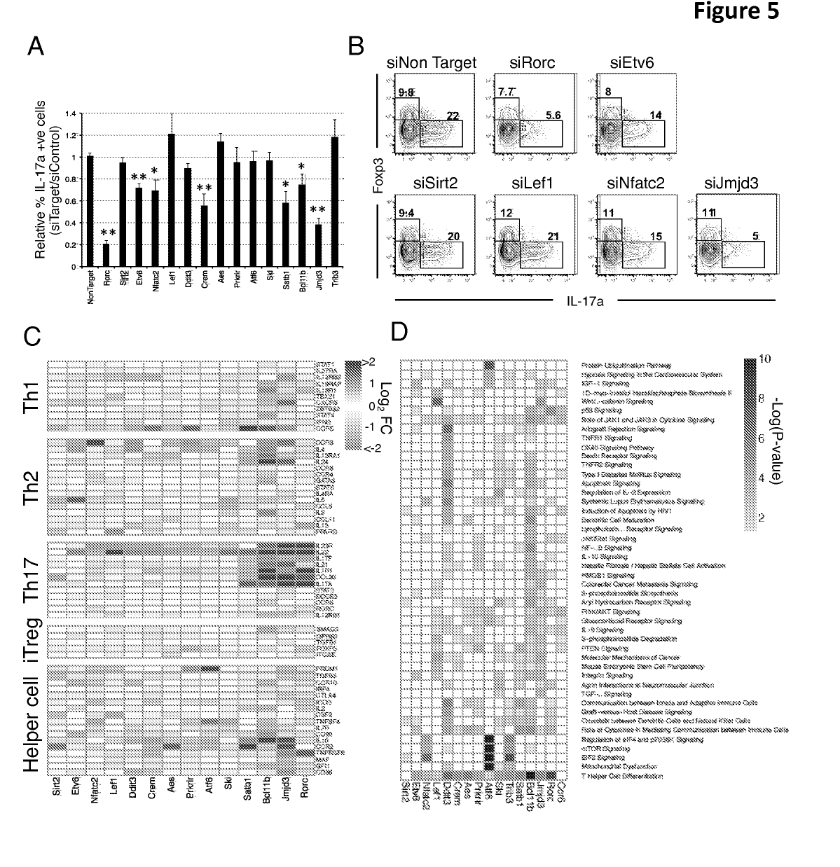

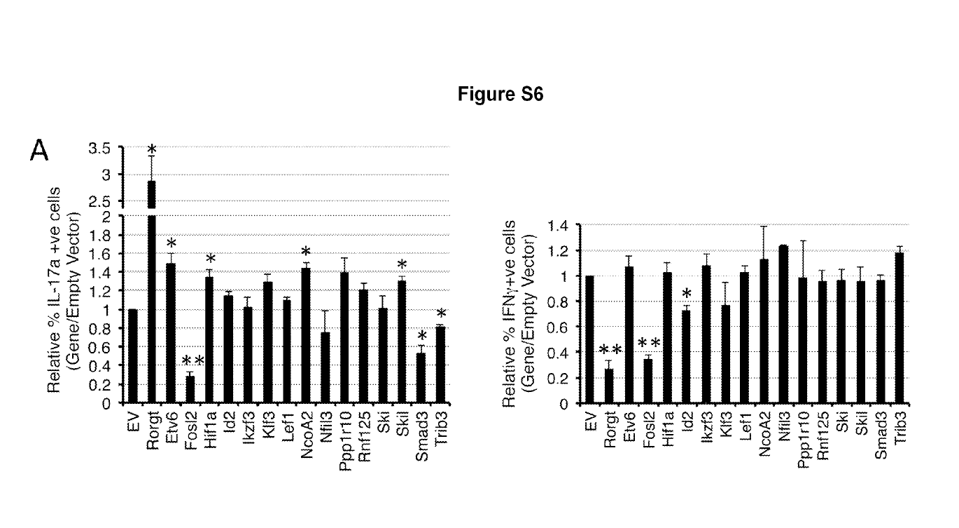

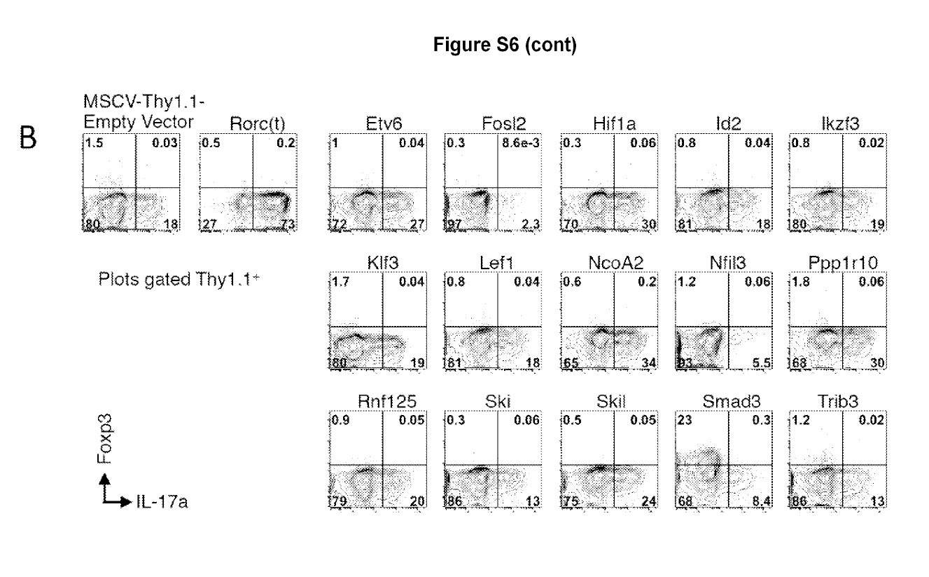

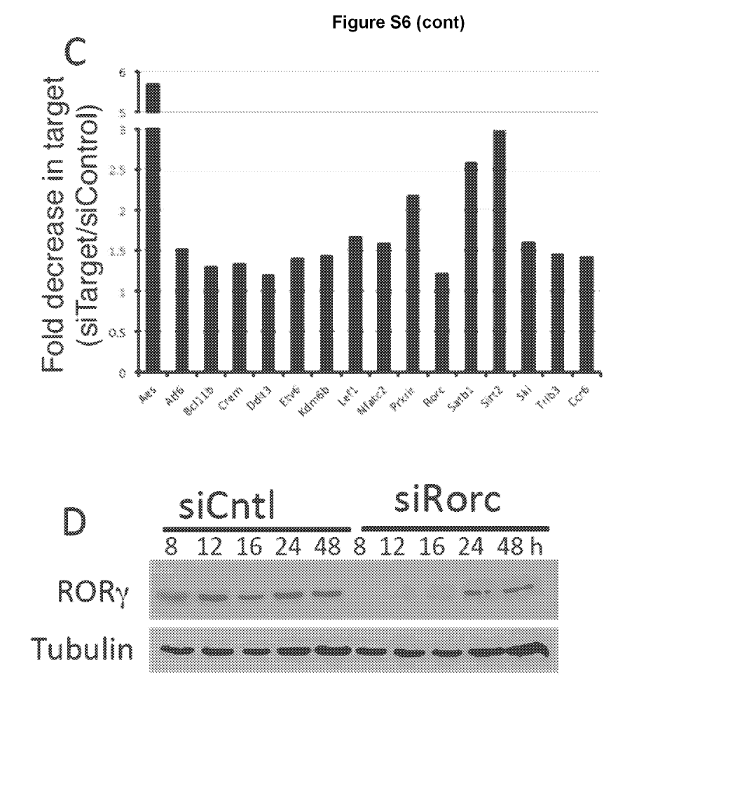

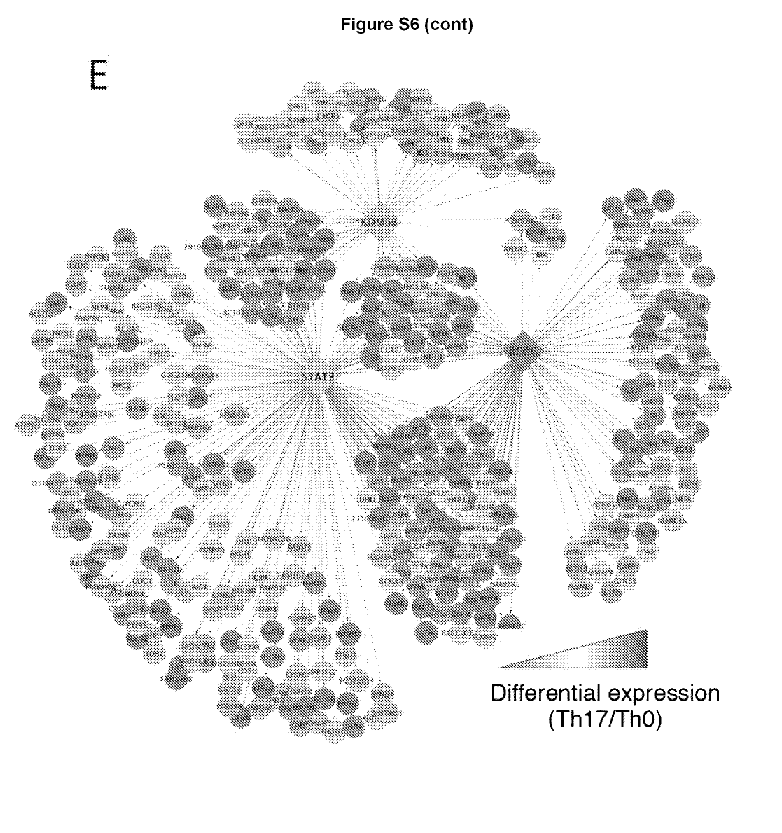

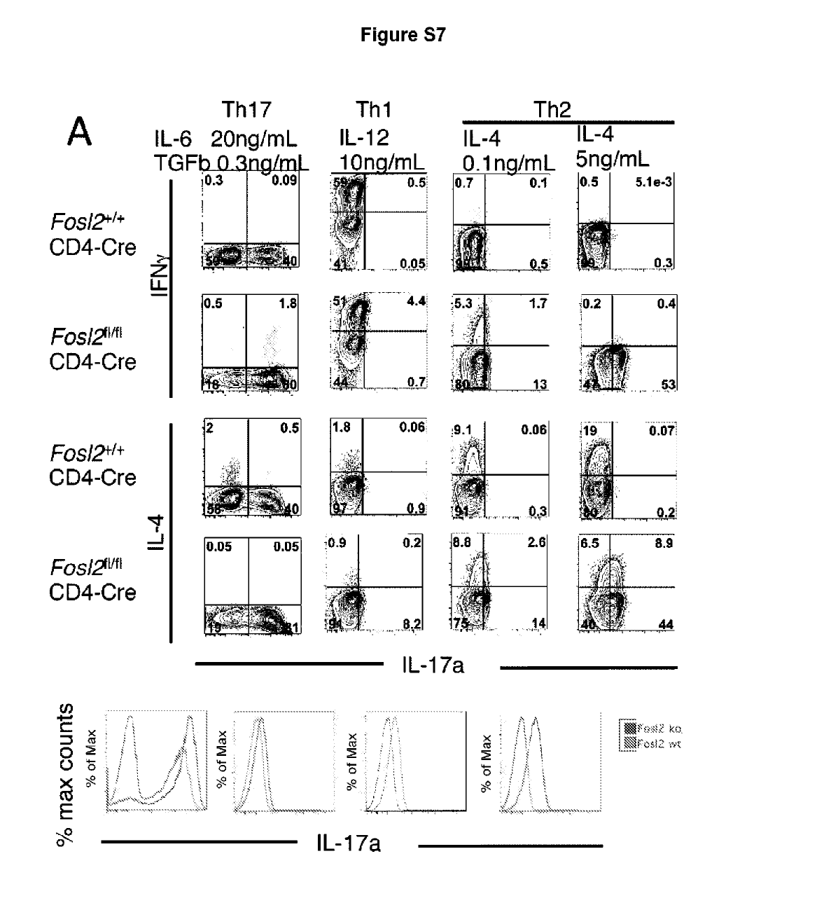

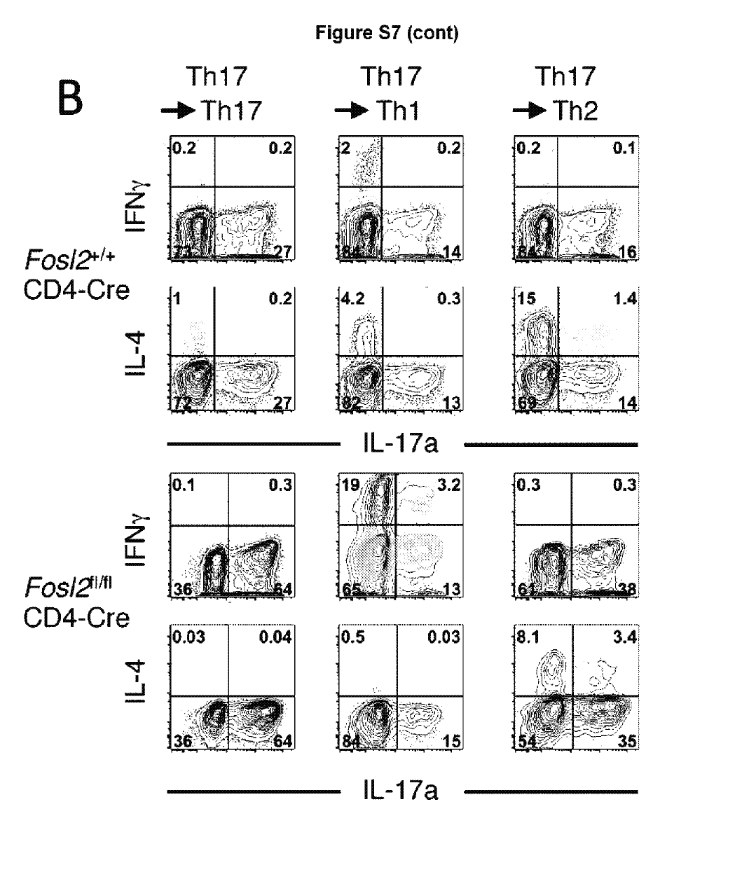

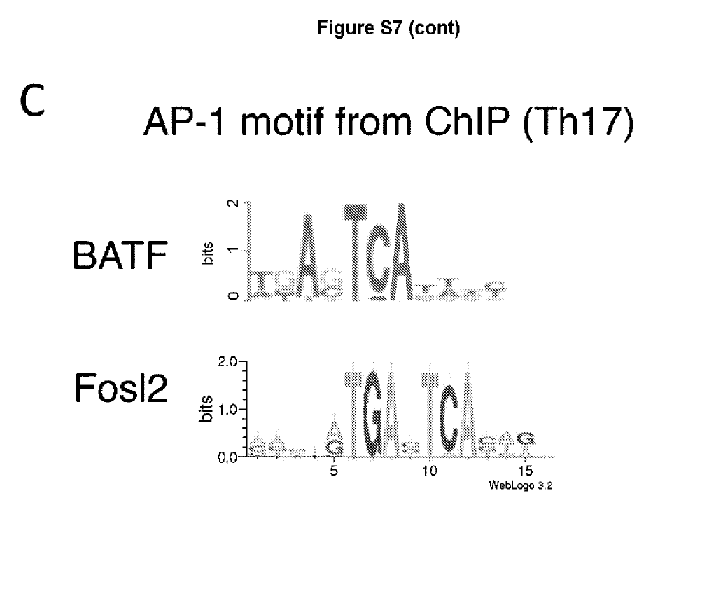

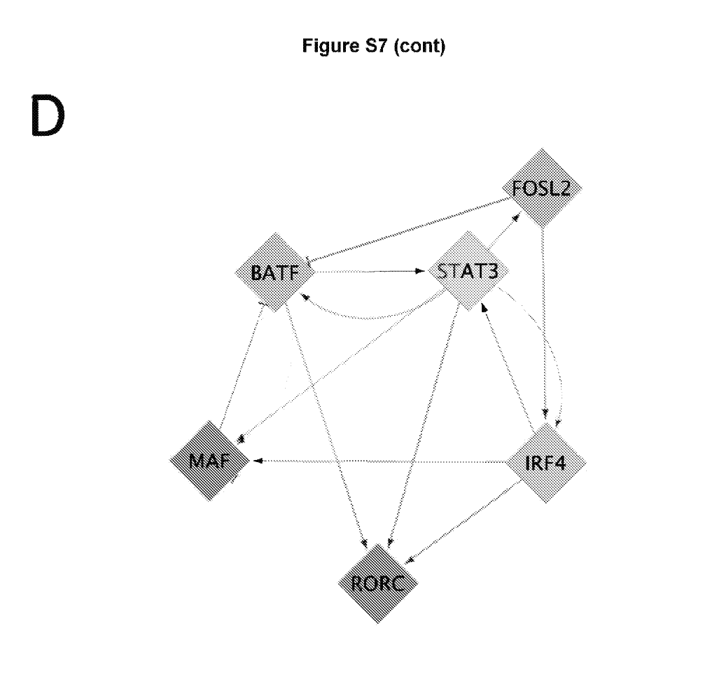

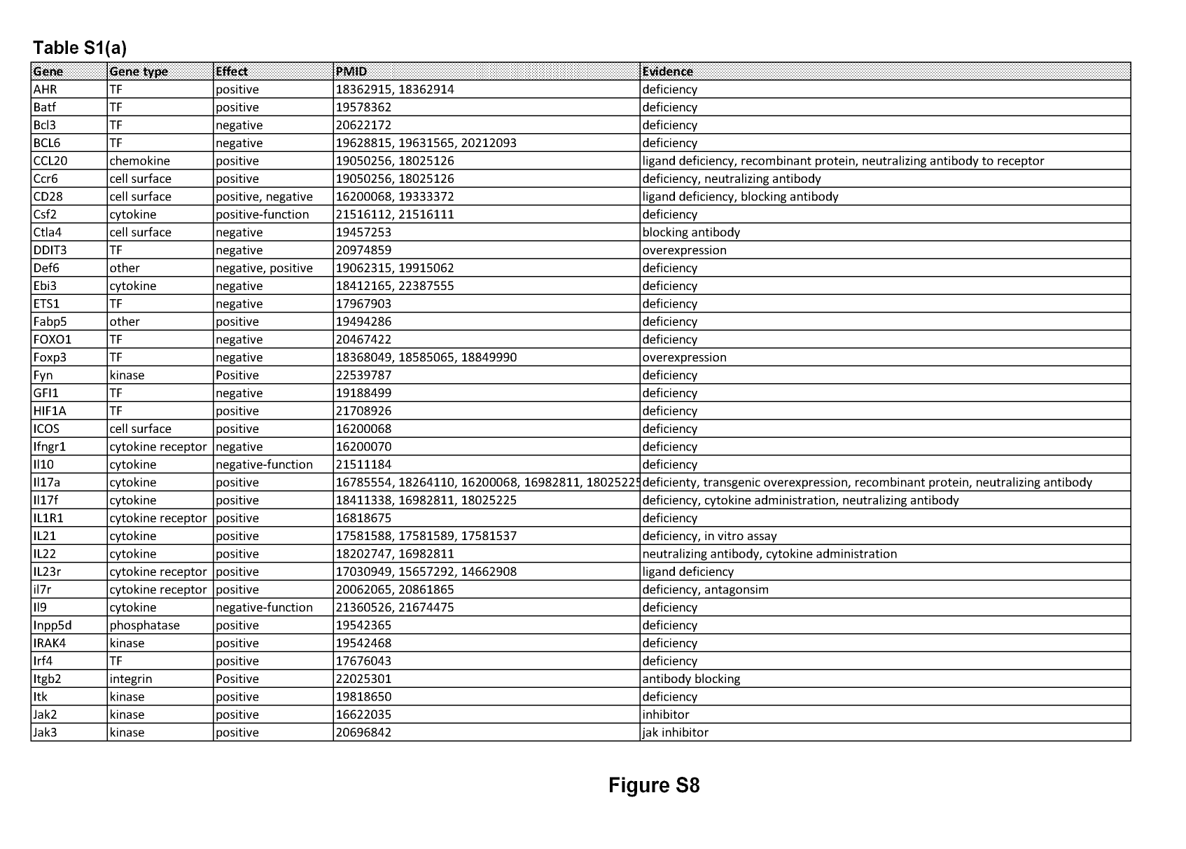

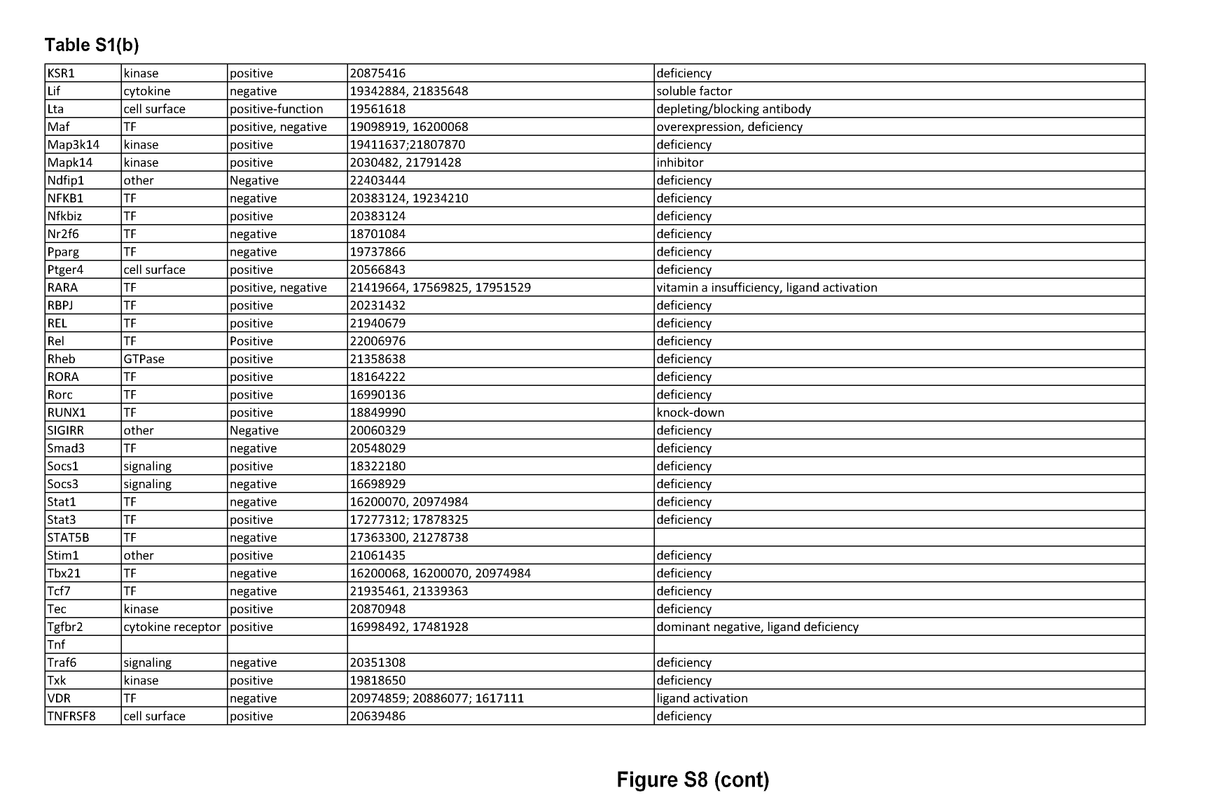

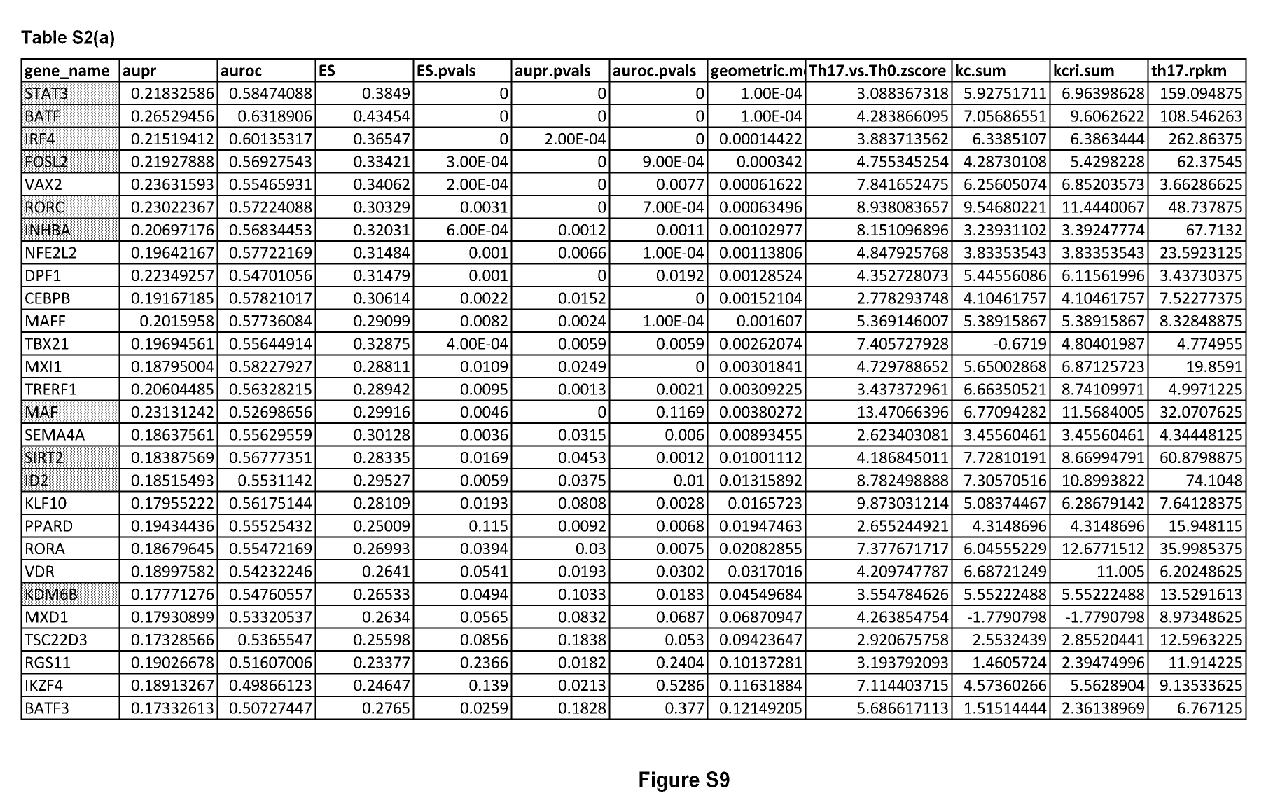

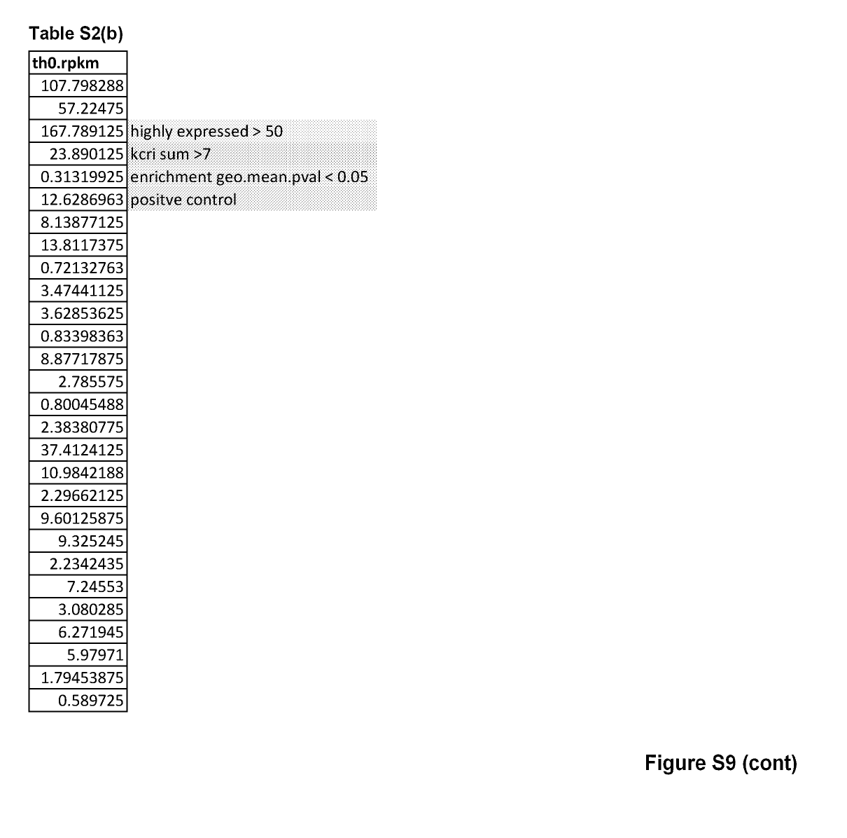

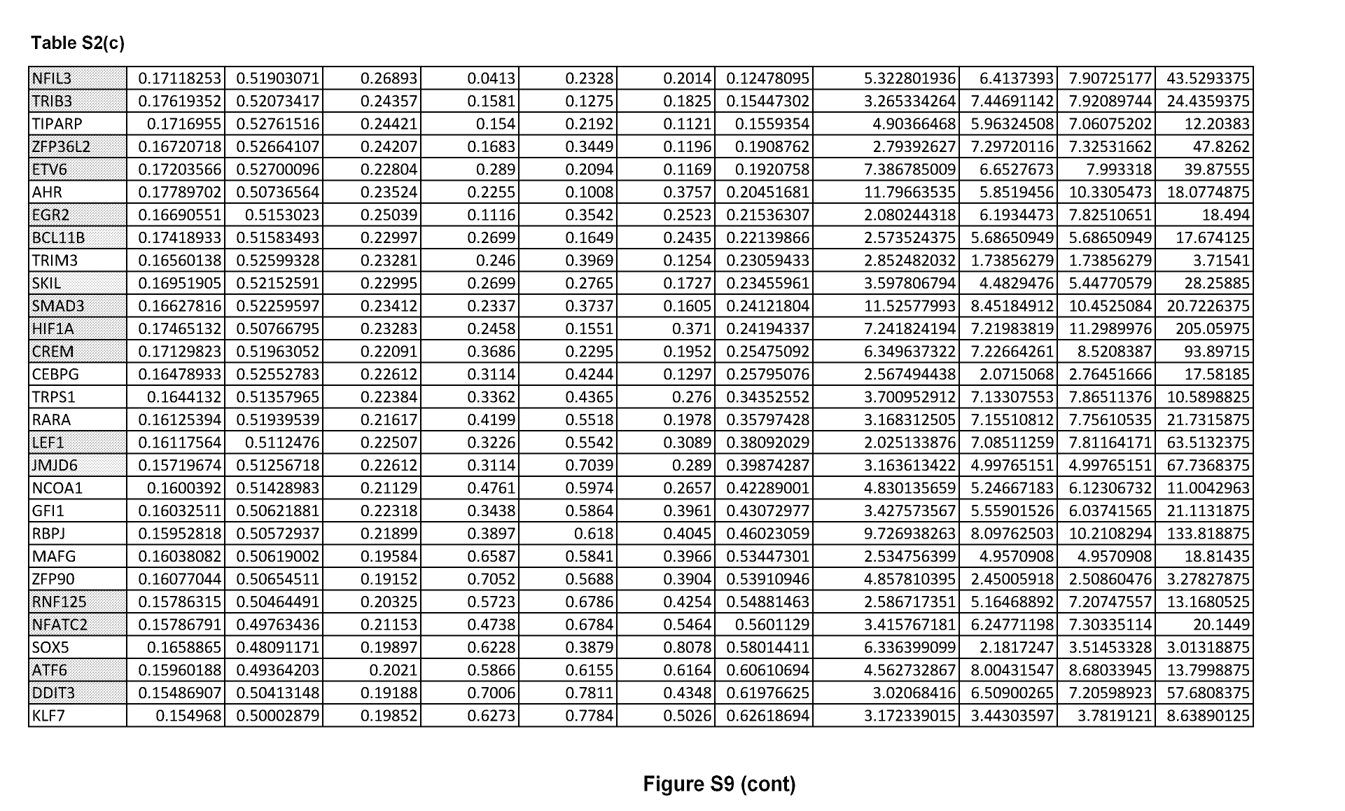

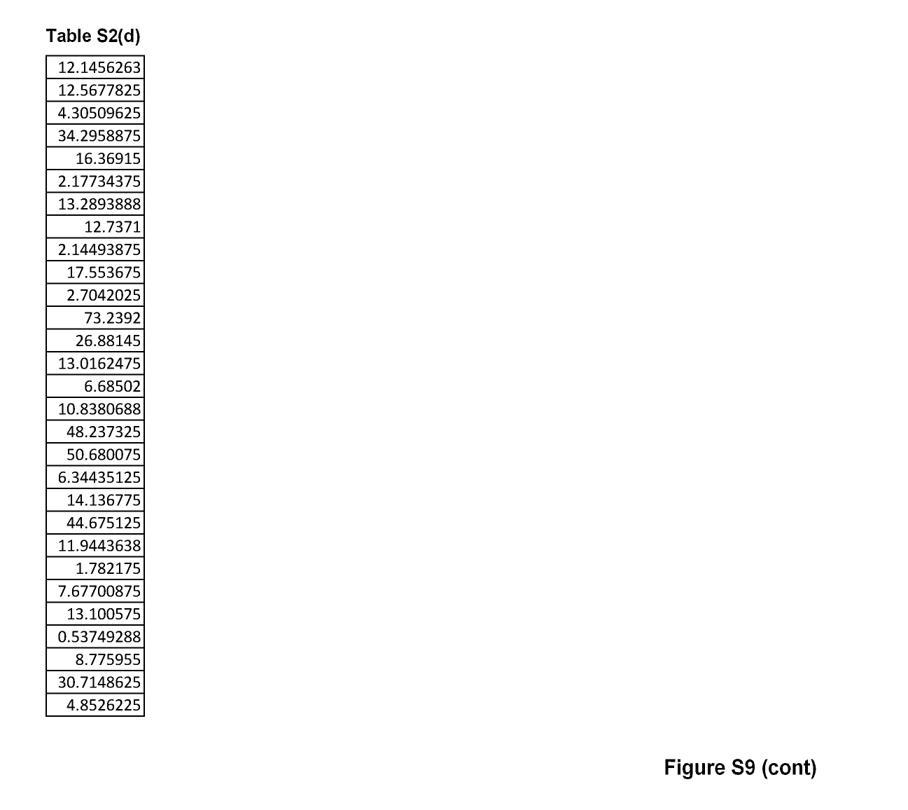

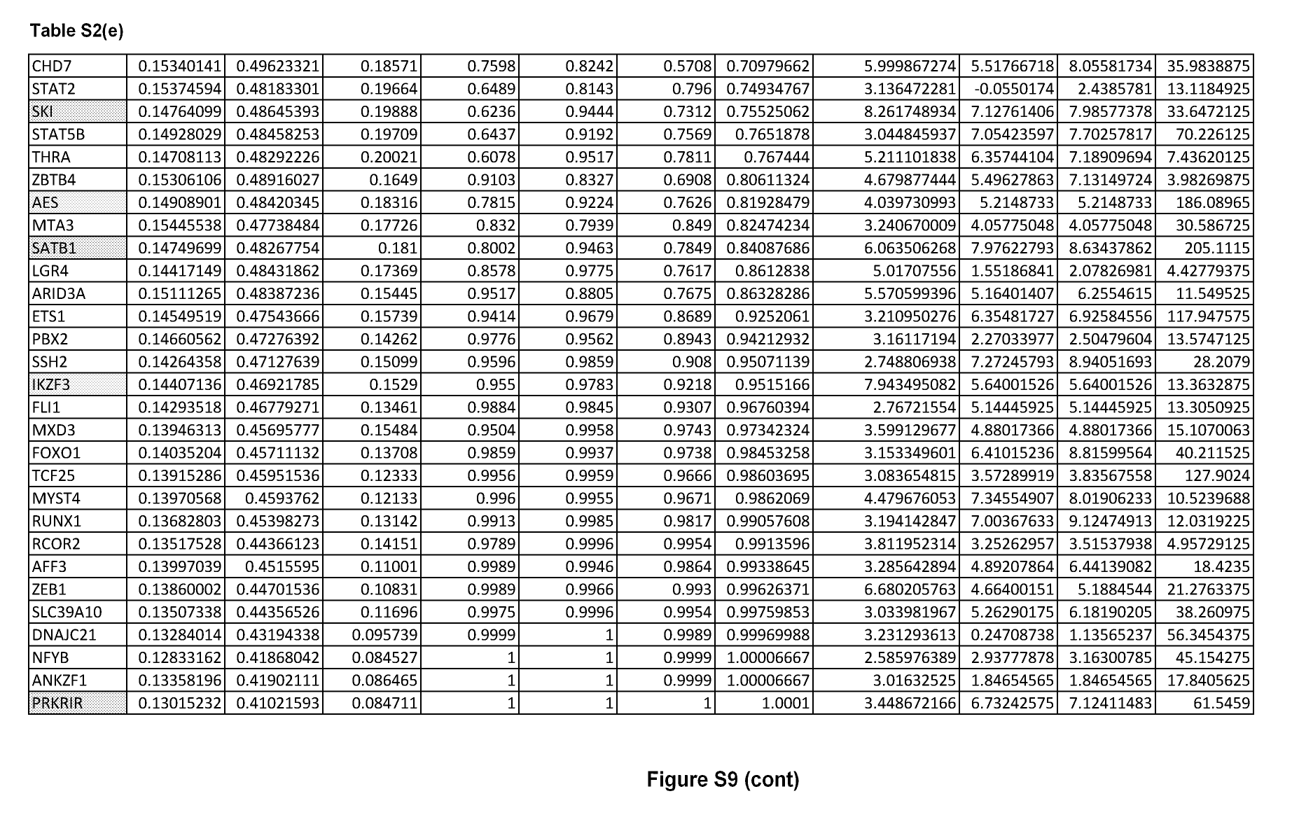

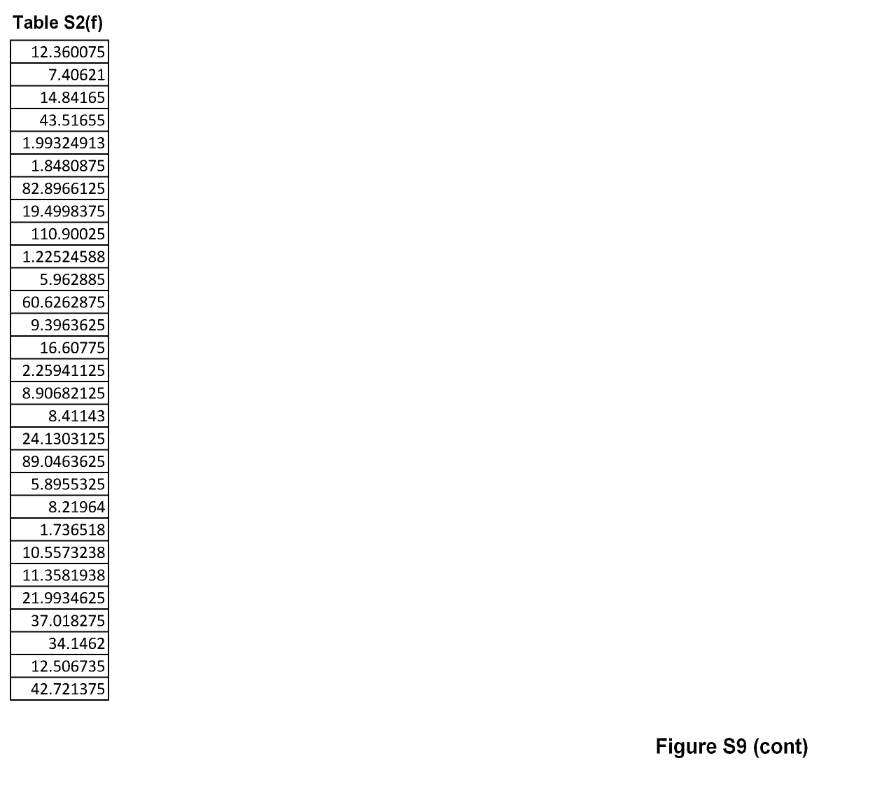

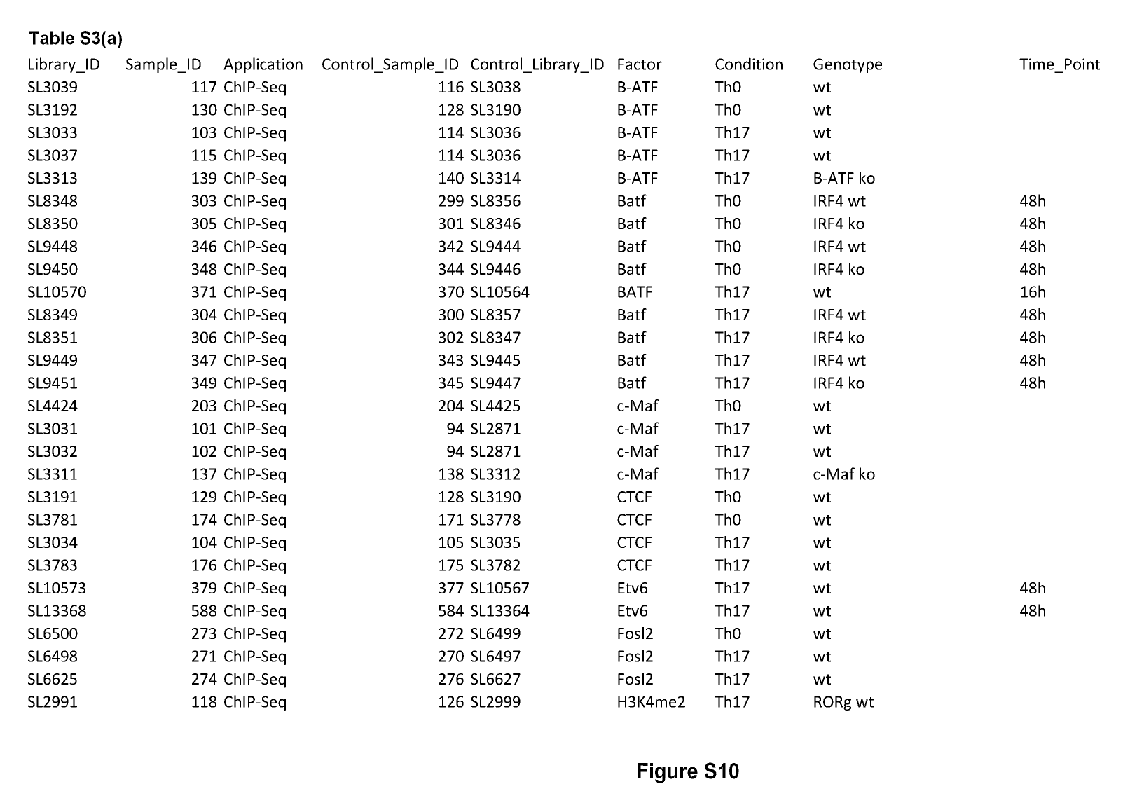

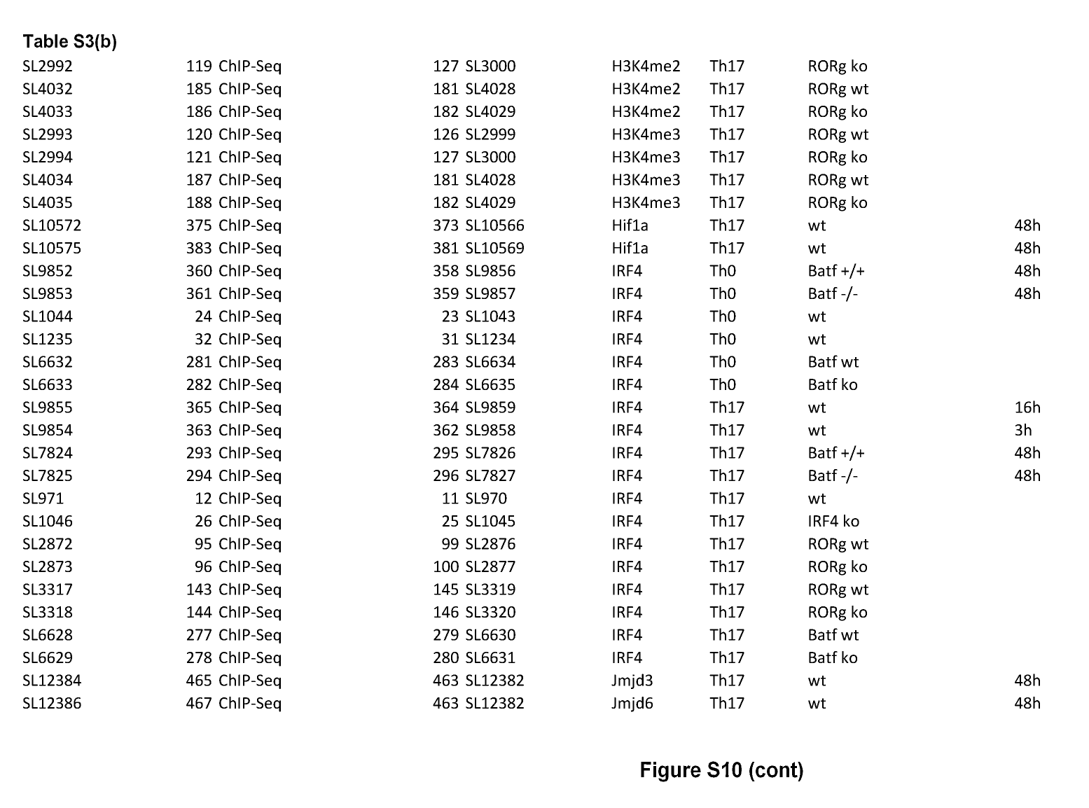

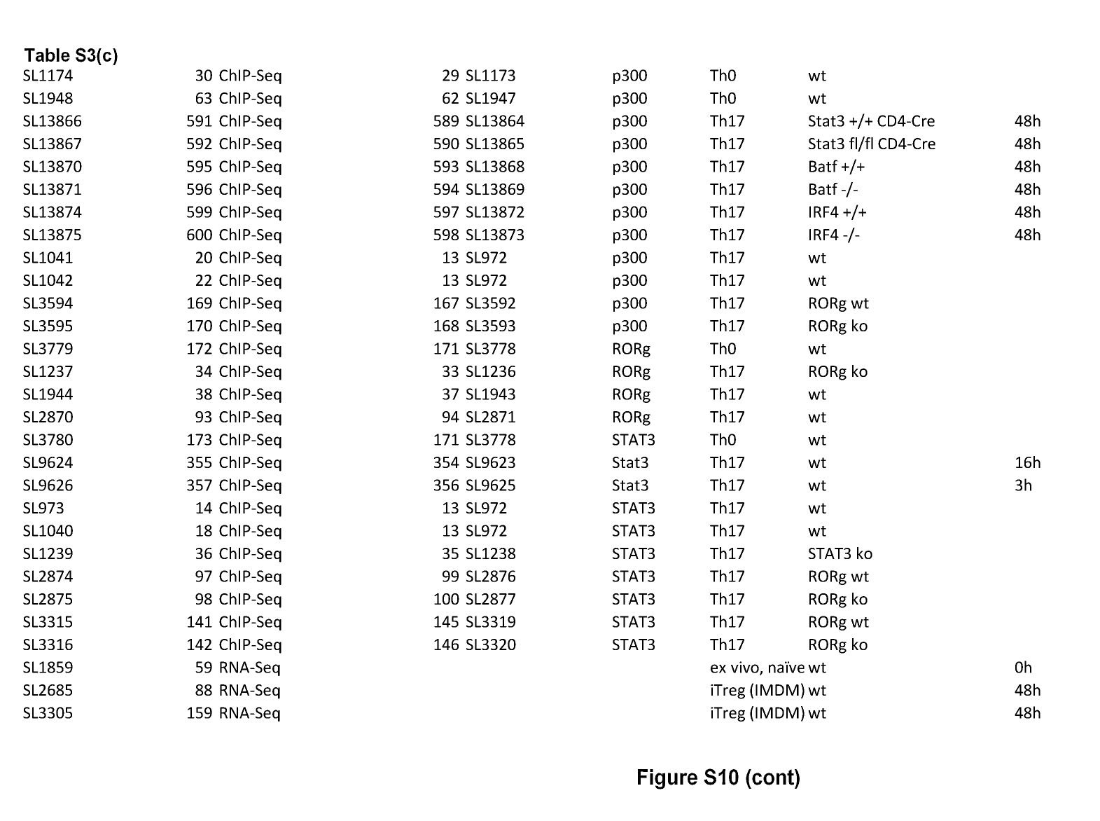

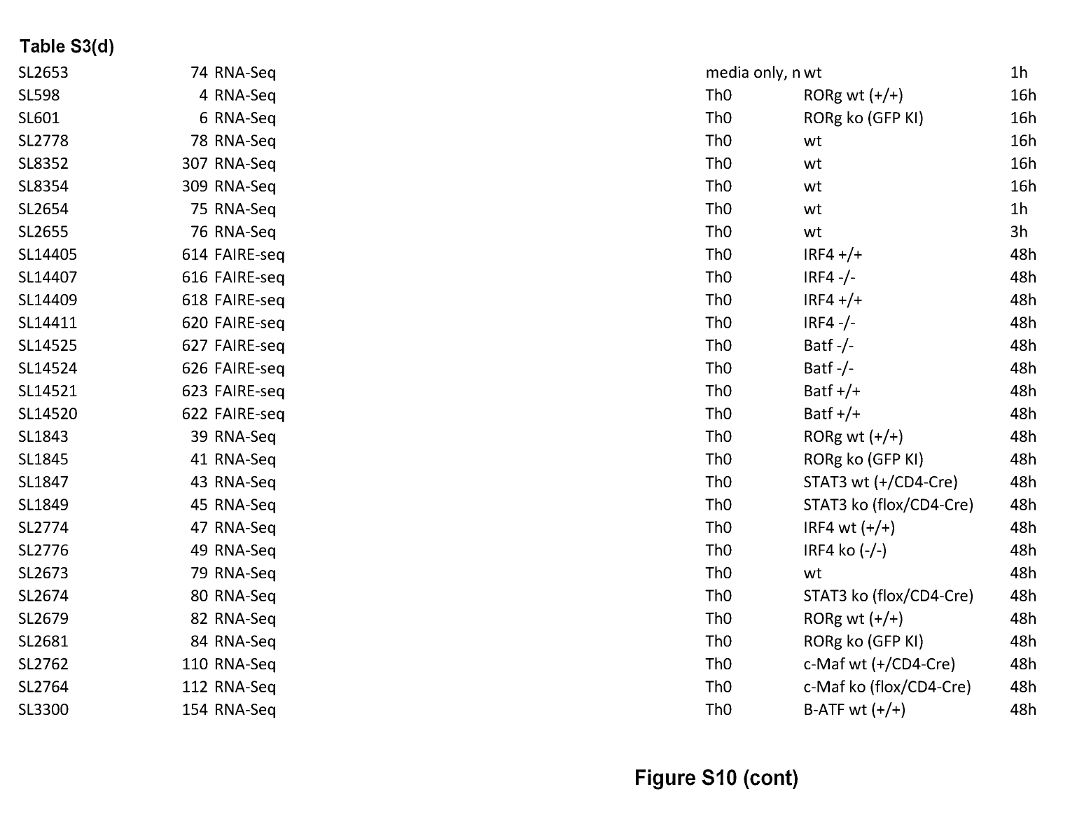

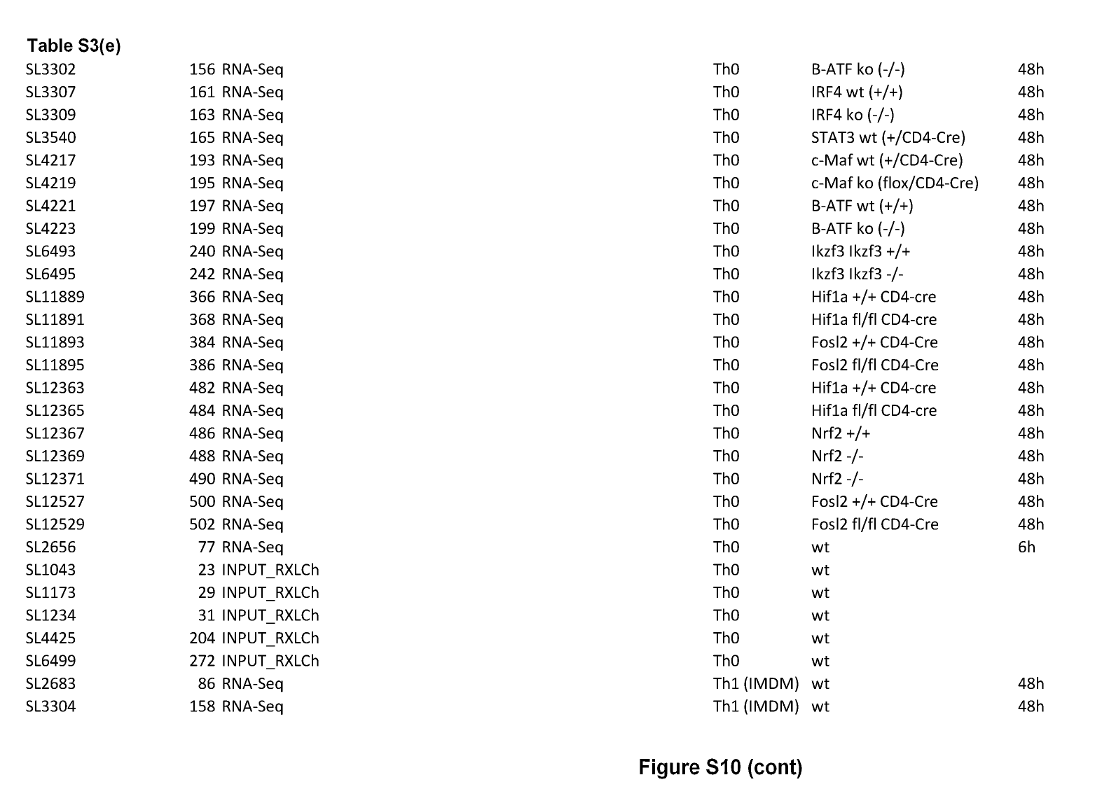

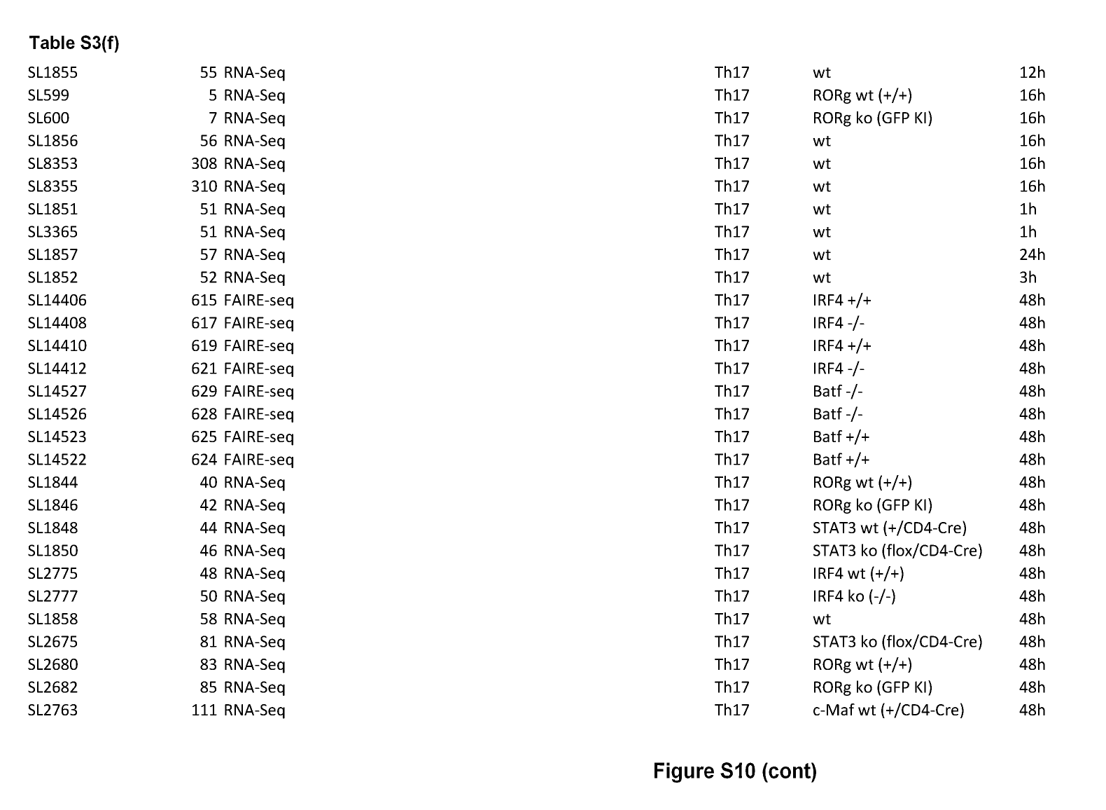

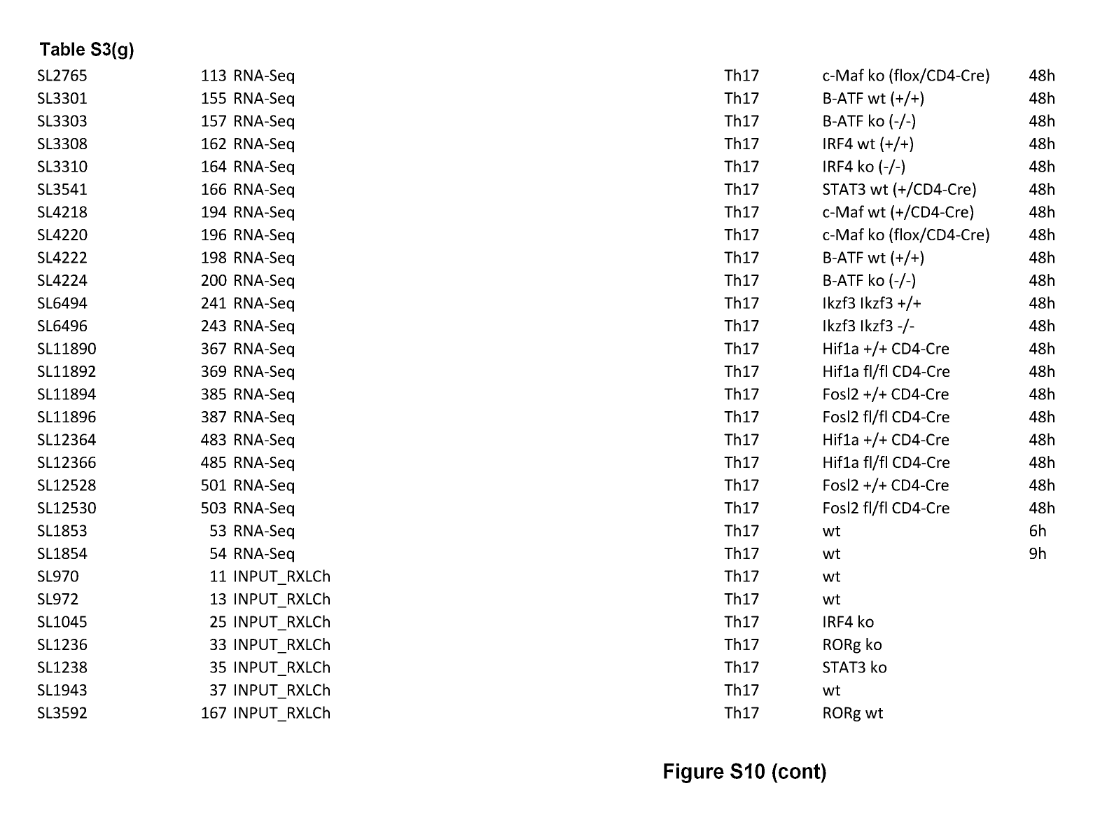

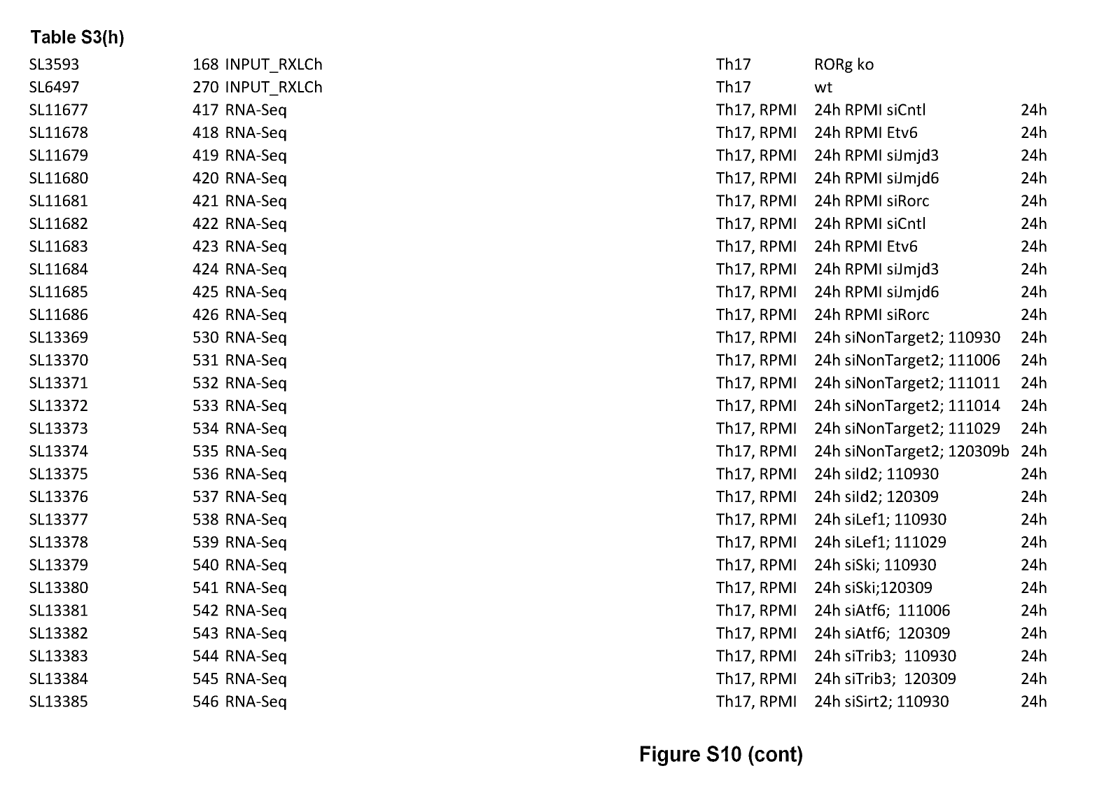

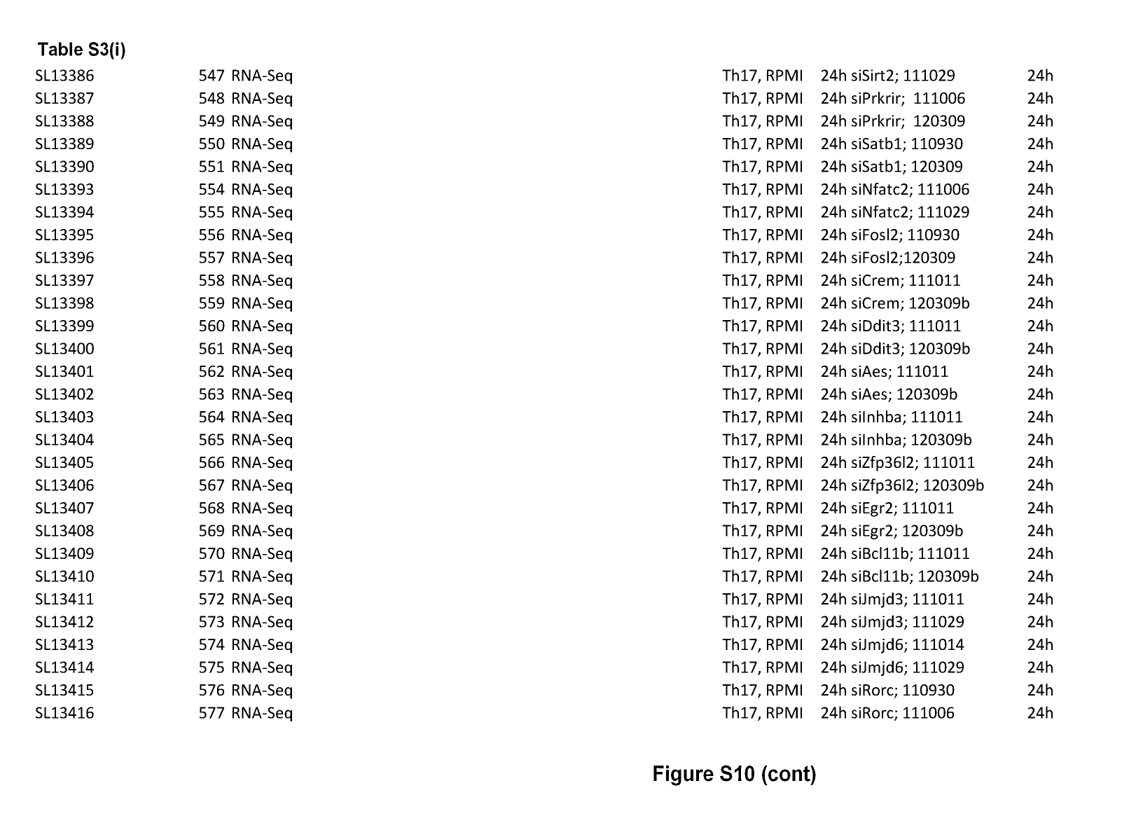

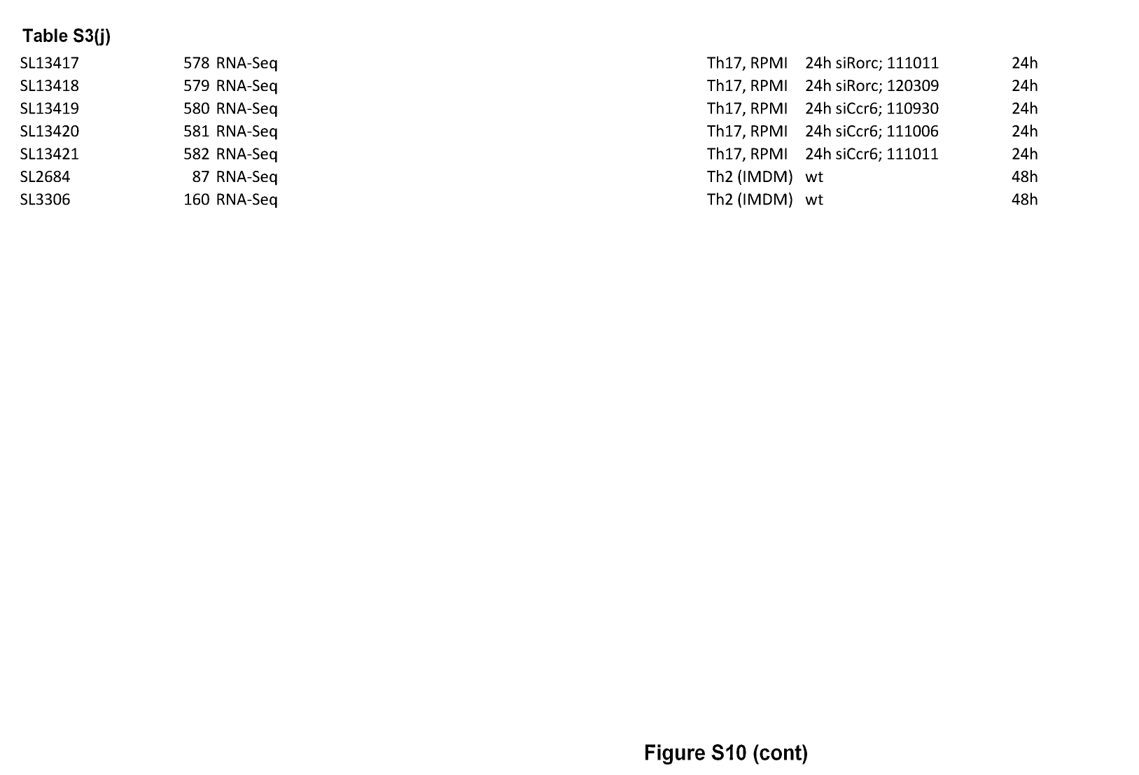

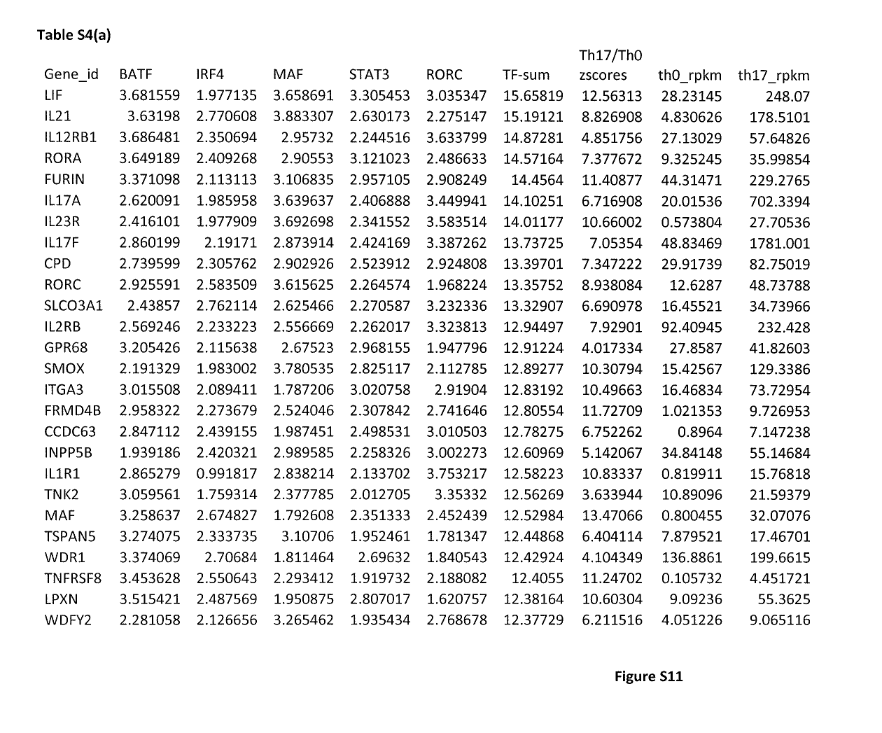

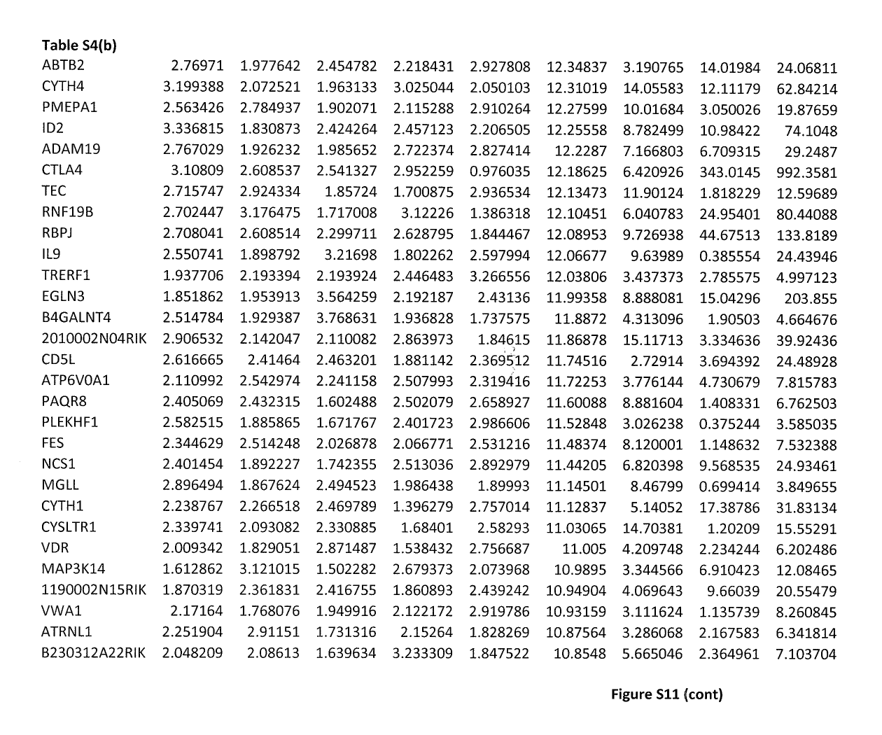

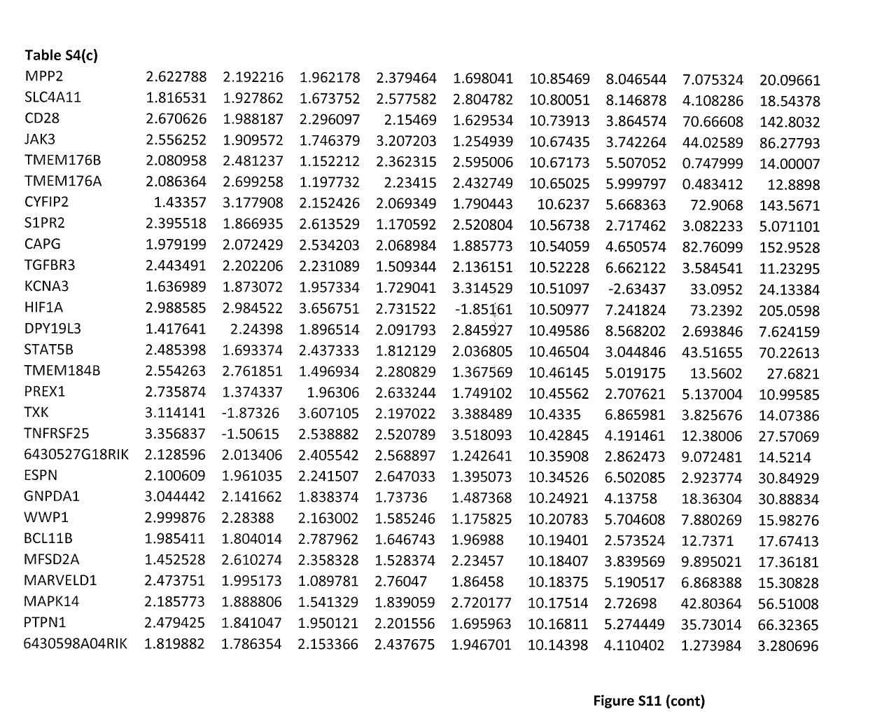

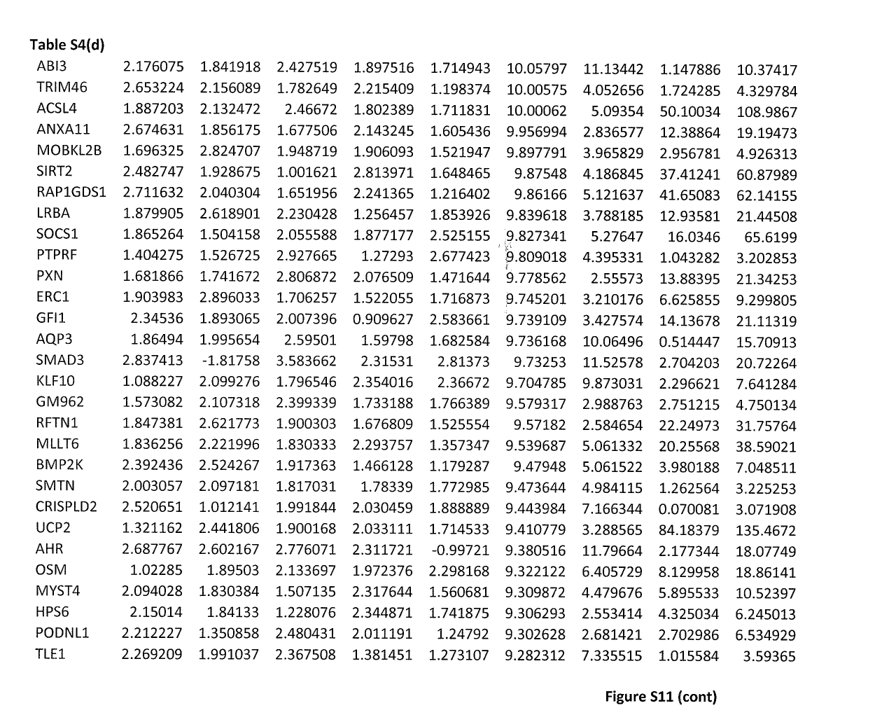

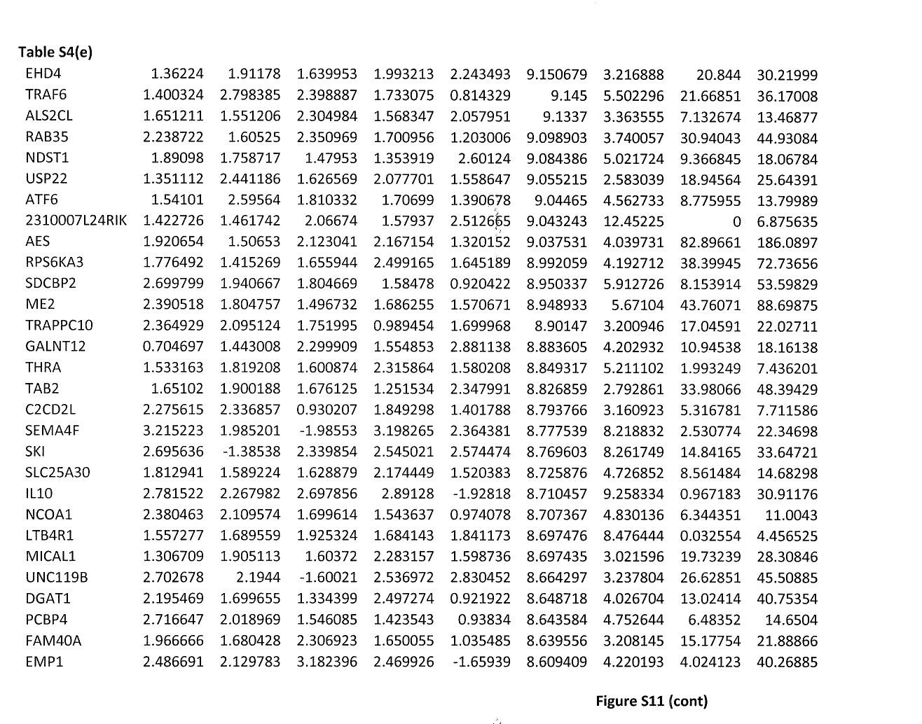

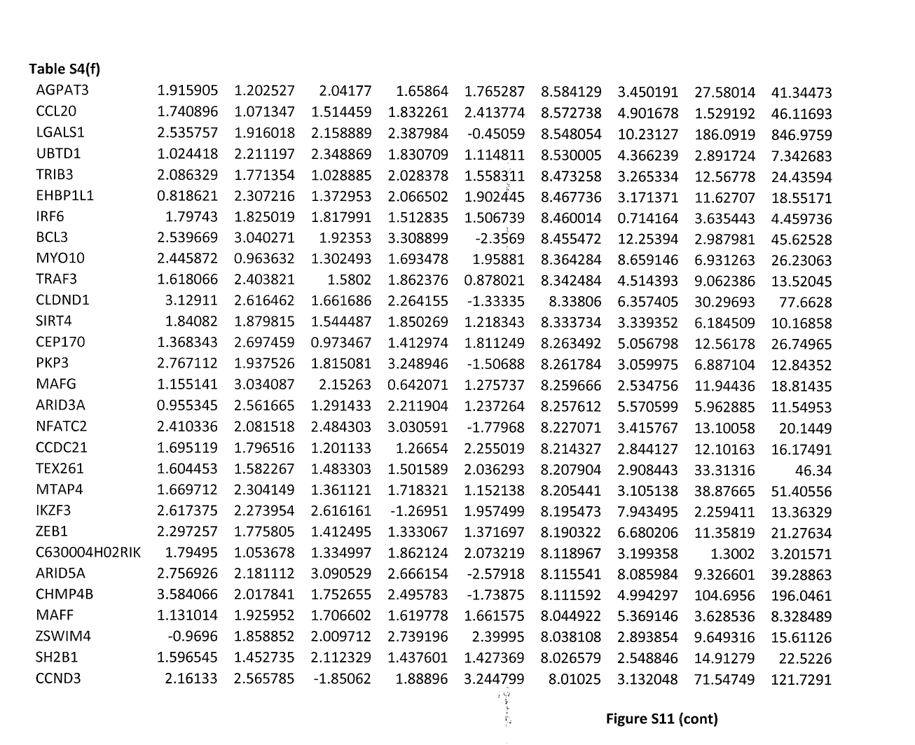

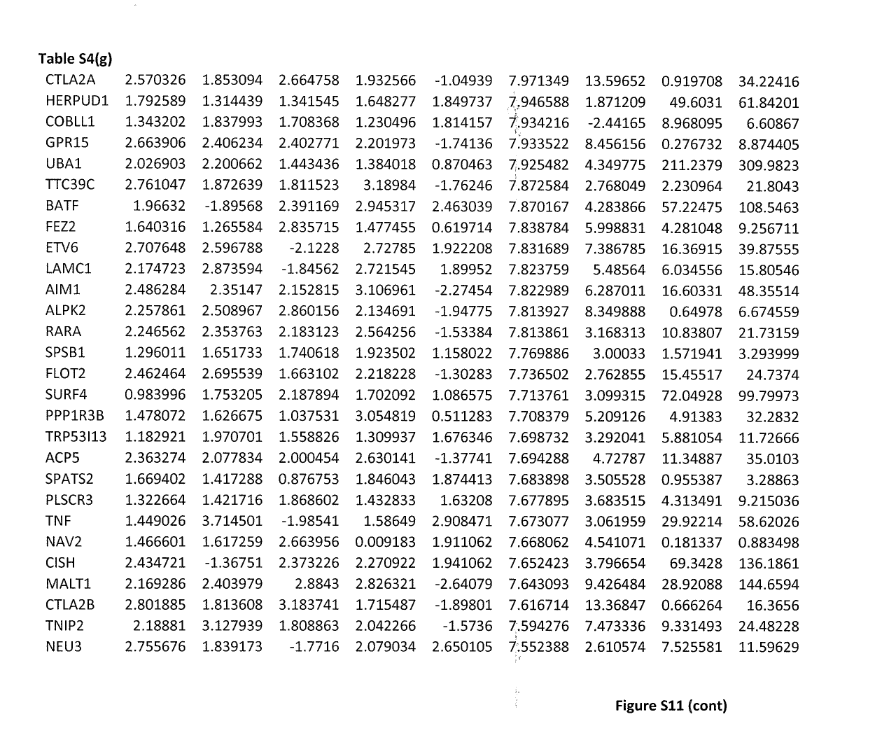

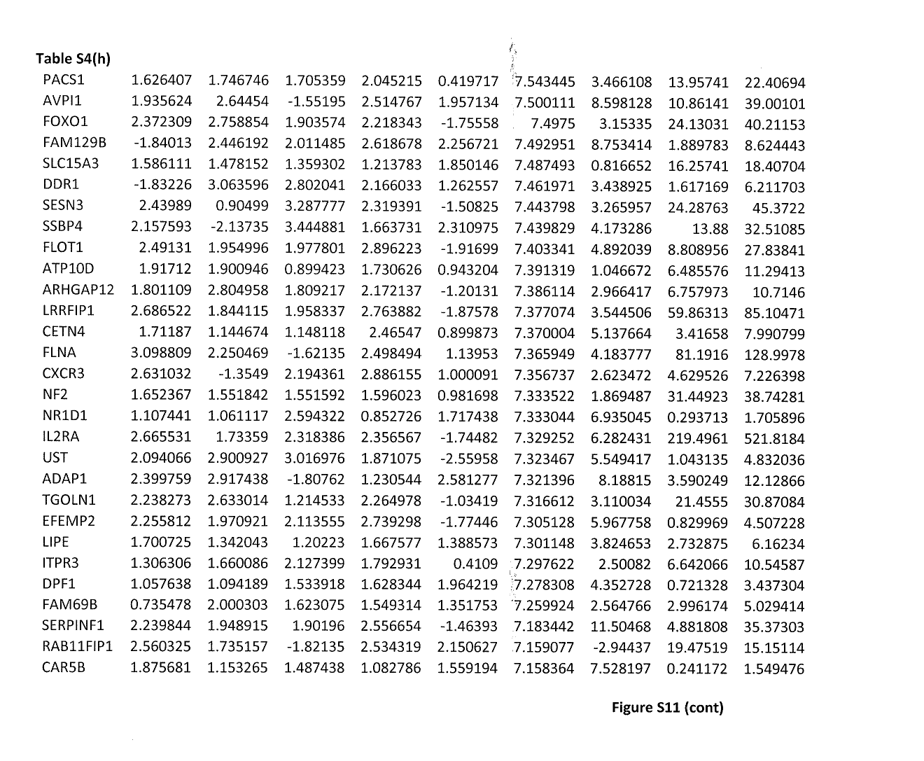

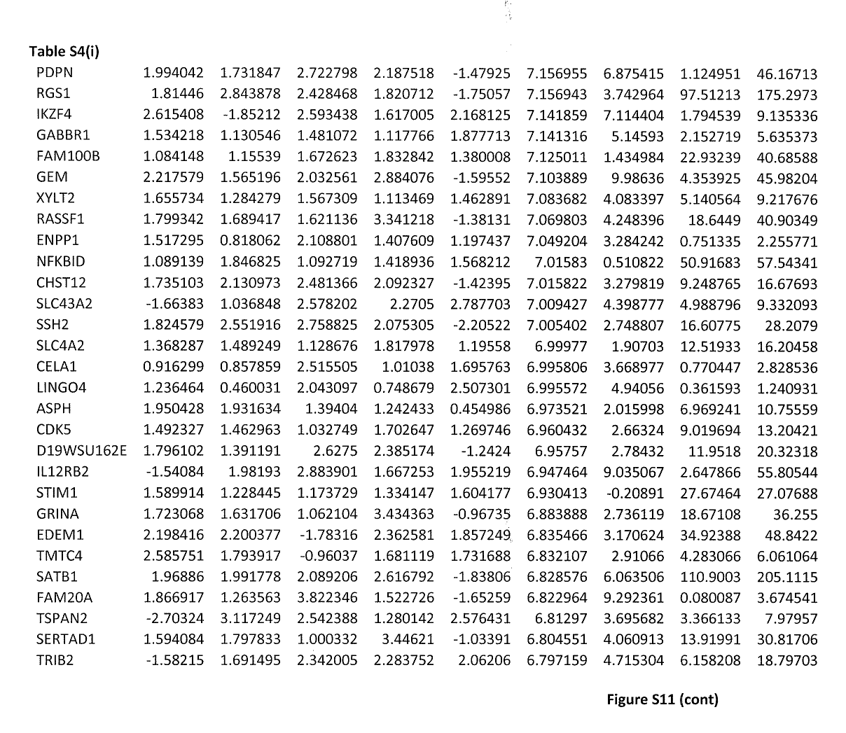

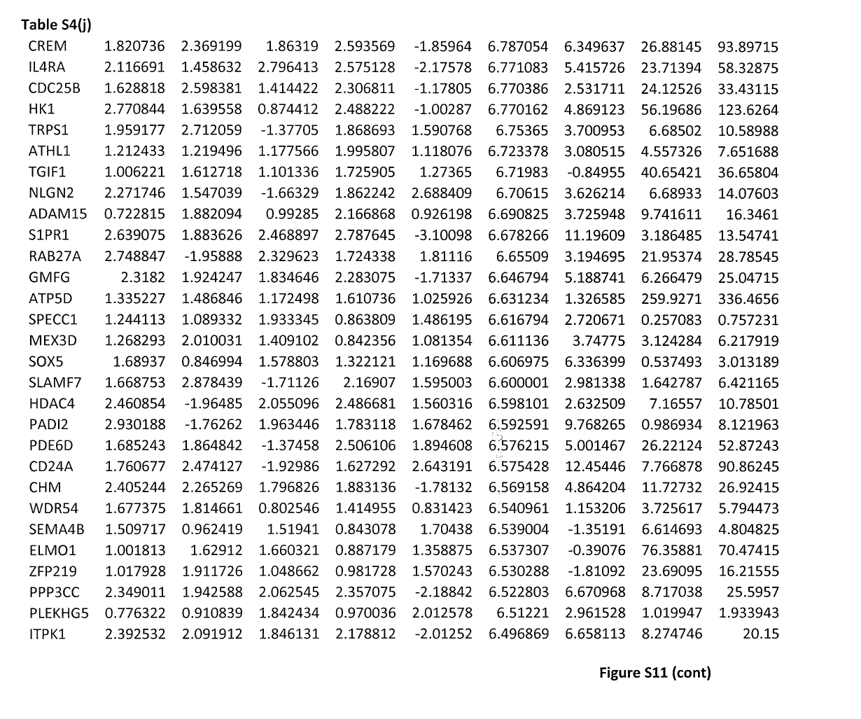

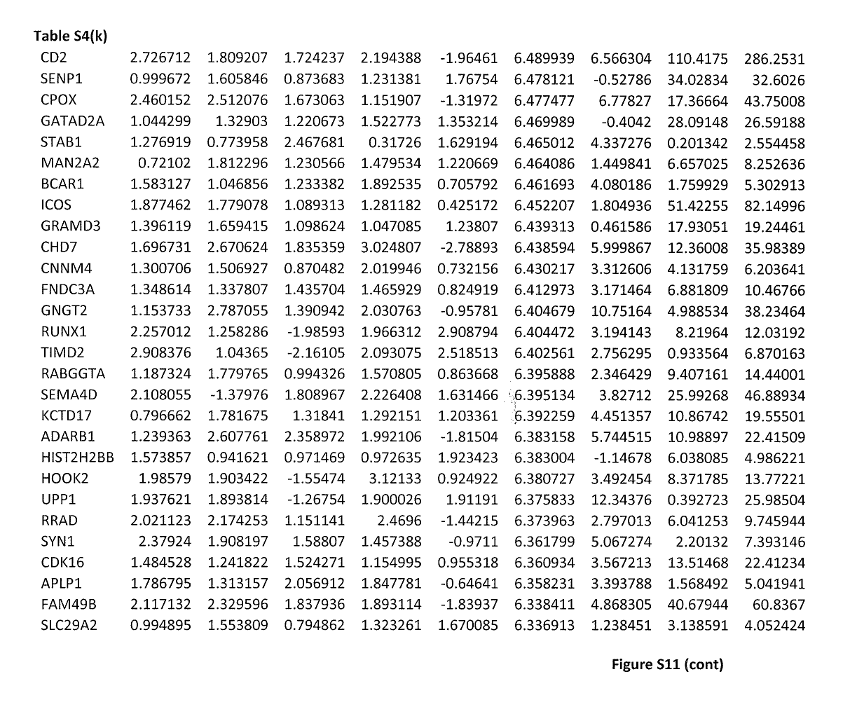

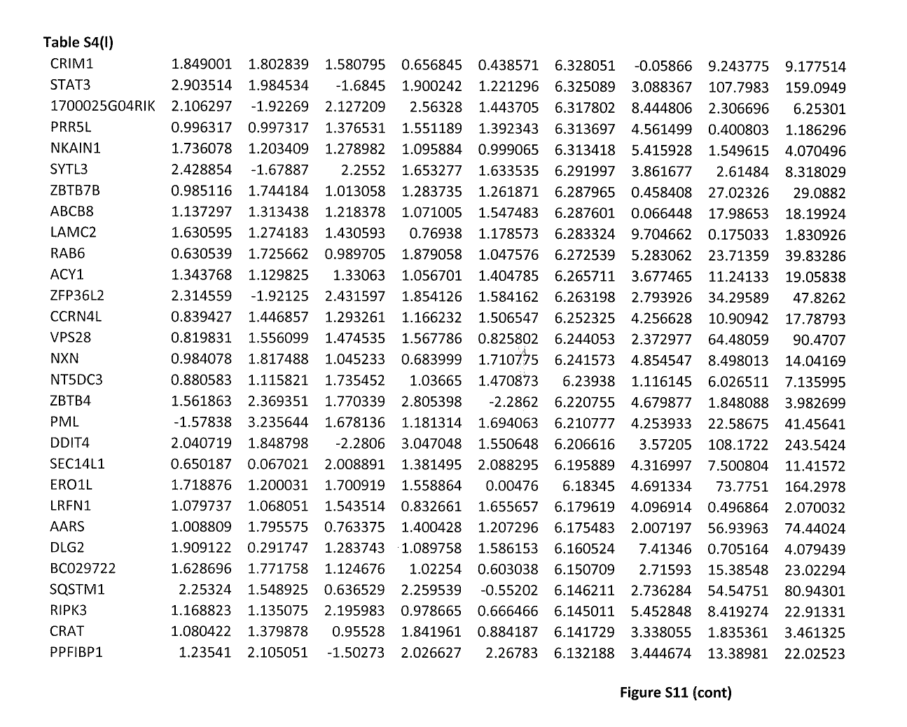

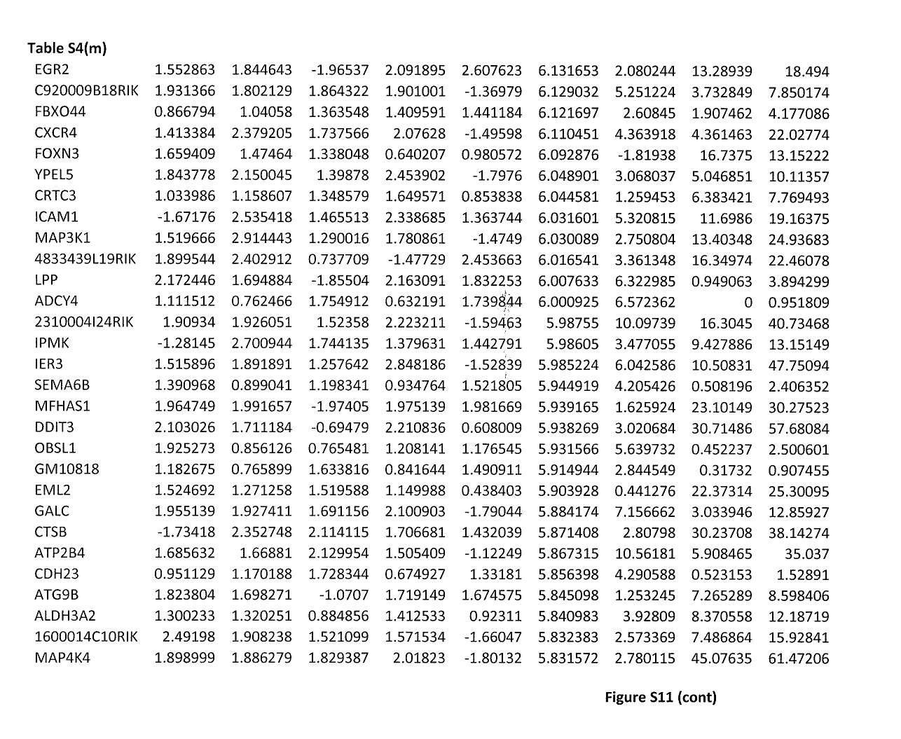

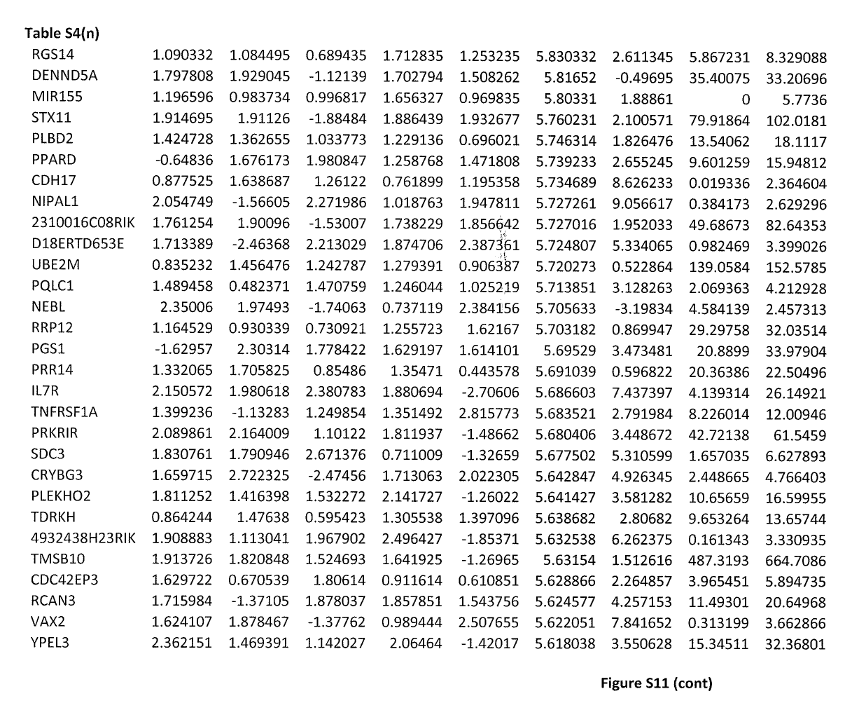

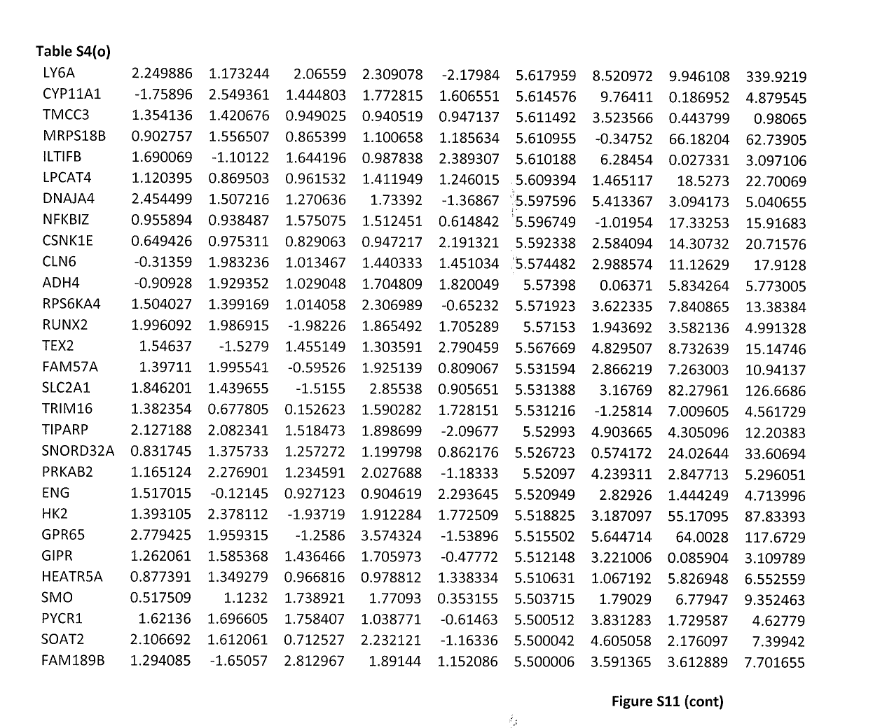

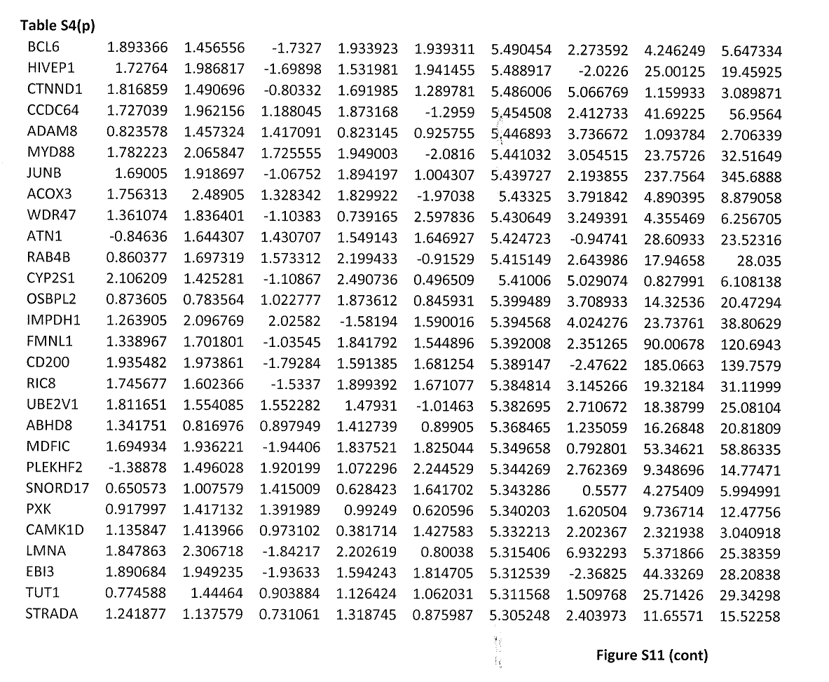

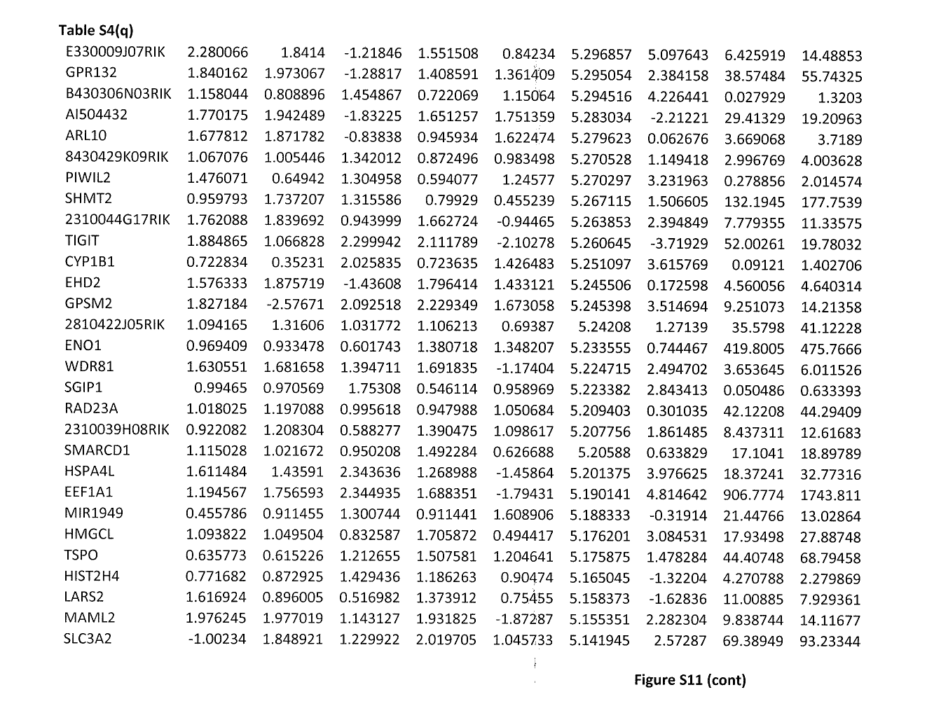

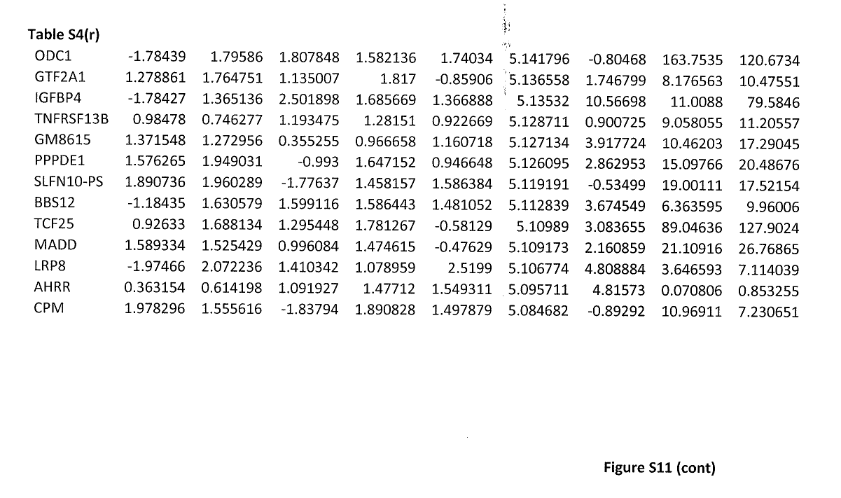

A. Proximal binding of BATF and IRF4. Distribution of the distance between ChIP peak summits for pairs of TFs in 5-TF pCRMs. Boxplot: as in FIG. 1; points=outliers.
B. Co-immunoprecipitation of BATF, but not STAT3, with IRF4 in 48 h Th17-polarized cultures. Ethidium bromide (EtBr) disrupts DNA-protein interactions. IP: immunoprecipitating antibody; IB: immunoblotting antibody. Representative of two experiments.
C. MEME-ChIP motifs identified in 4 sub-types of pCRMs as indicated. The AP-1 and ISRE consensus is recovered in regions singly occupied by BATF, and IRF4, respectively. A new AP-1-ISRE composite motif comprising an AP-1 site (TGA(C/G)TCA) adjacent to an ISRE half site (GAAA; boxed region) is only recovered at pCRMs occupied by BATF and IRF4. ISRE half site orientation differs according to whether or not there is a 4 bp interval. The fraction of pCRMs for which the motif is found is indicated.
D. Genome-wide interdependence of IRF4 and BATF co-occupancy in Th17-polarized cells. Scatter plots display the fold change in ChIP-seq reads vs. significance. Top: IRF4 in Batf wt vs. KO. Bottom: BATF in Irf4 wt vs. KO. Differences in ChIP-Seq reads displayed for 3 relevant pCRM sub-types: BATF or IRF4 alone (green); BATF and IRF4 (orange); and BATF, IRF4, plus additional TFs (purple). Distribution of fold changes of wt vs. KO occupancy are displayed for proximal and distal pCRMs; boxplots as in FIG. 1C. RPM; reads per million. ** Significant at P-value<0.001, Kolmogorov-Smirnov test.
E. Reciprocally reduced occupancy of BATF and IRF4 at the Cd28 locus in Th17-polarized cells deficient for IRF4 and BATF, respectively. ChIP-seq tracks were normalized for library size.

FIG. 3. TF combinatorial interactions specify the Th17 lineage.

A. High confidence regulatory edges (FDR<10%; based on 10,000 simulations) focused on five core TFs identify direct (ChIP-seq) and functional (KO RNA-seq) regulatory targets (visualized using Cytoscape). Boxed inset displays the regulatory interactions between core Th17 TFs. See network legend for visualization scheme.
B. Expanded view of highly regulated nodes with four to five core regulatory inputs, grouped based on general functional categories.
C. Regulatory interactions shared by STAT3, IRF4, BATF, and RORγt highlighting different aspects of RORγt transcriptional function. Attenuation: RORγt repression targets that are up-regulated in Th17 cells; Reinforcement: Activation targets that are up-regulated in Th17 cells; Essential: targets having a two-fold change in RORγt KO differential expression and KO H3K4me3 ChIP.
D. Targets for single TFs are enriched for pathways in multiple functional categories (i). Targets of multiple TFs (increasingly regulated by 2, 3, or 4+5 TFs) are selectively enriched for pathways related to T helper differentiation and effector function (ii). Analysis performed using the Ingenuity analysis tool (IPA) is presented as a heat map of enrichment p-values.

FIG. 4. Network model performance and validation.

A. Schematic for integration of four genomics and systems data types (K, C, R, and I) using a rank combined (RC) approach, resulting in the KC and KCRI networks.
B. Performance measured as aucPR values indicating enrichment of literature-curated Th17 genes in networks derived from all possible data combinations. Points indicate single TF predictions (e.g. BATF→target) and bars indicate TF sum predictions (i.e. [BATF+IRF4+STAT3+MAF+RORγt]→target). Dotted line, reference performance for targets prioritized by differential expression (Th17 vs. Th0) and, dashed line, for random.
C. Gene Set Enrichment Analysis (GSEA, Broad Institute) for the top-performing KCRI network. (i) The ranked list of TF sum targets recovers Th17-relevant genes with a maximal enrichment score (ES) of 0.86 out of 1 (red line); random (gray line). (ii) Vertical red lines indicate where in the ranked list literature-derived genes were recovered. (iii) Summed TF score distributions for KCRI (red line) and random (gray line).
D. The KCRI network selectively recovers GWAS SNP-linked genes for Th17-implicated inflammatory diseases. Recovery of SNP-associated disease genes is measured in terms of aucPR for TF sum predictions (gray bars) and single TF predictions (points).
E. Core TF networks for genes associated with GWAS of Crohn's disease and Type 2 Diabetes. The KCRI network recovered 24 out of 80 Crohn's disease genes (p-val=10⁻⁷), and 9 out of 84 Type 2 Diabetes genes (p-val=0.29). Network display is as in FIG. 3. P-values calculated by Fisher's exact test.

FIG. 5. Identification of novel Th17 regulators.

A. siRNA knock-down screen of candidate TF function in Th17 differentiation. Percent of IL17A-producing cells relative to the control siRNA condition for knock-down cultures analyzed at 24 h of Th17 polarization. Error bars=SD of two experiments conducted in triplicate. *P<0.05 and **P<0.01, T-test.
B. Flow cytometric analysis for Th17-polarized cells transfected with siRNAs for the indicated gene targets.
C. TF candidates influence the expression of immune-modulatory genes. Heat map of log 2 fold change in expression of T helper signature genes in the siRNA knock-downs relative to non-targeting control for 24 h Th17 polarization cultures.
D. Shared and unique functions of novel Th17 TF regulators. Heat map of Ingenuity pathway enrichment (IPA, p-value<0.01) for candidate TF-dependent genes.

FIG. 6. Fos12 regulates loci critical to lineage identity and function.

A. Fos12 negatively regulates IL-17A expression. Flow cytometric analysis for naïve Fos12 wt and KO CD4 T cells cultured as indicated for 3 days. Representative of 4 experiments.
B. Conditional deficiency of Fos12 in CD4 T cells reduces the severity of EAE. Top panel shows clinical scores (mean±s.e.m.) for CD4-cre mice with wt or conditional Fos12 alleles (*P<0.05 and **P<0.01, T-test). Bottom panel displays total numbers or percentages of CD4⁺ T cell populations isolated from spinal cord (mean±s.e.m.). Effector CD4⁺ T cells are defined as Foxp3⁻. Representative of two experiments.
C. Predominance of cytokine-producing Foxp3⁺ cells in mice during EAE. Flow cytometric analysis of re-stimulated CD4⁺ T cells isolated from the spinal cord and lymph nodes of mice on day 22 post EAE induction. Plots gated CD4⁺CD45⁺ cells.
D. Combinatorial core TF targets involved in T cell specification are highly regulated by Fos12. Network depiction is as in FIG. 3. Dashed lines: edges added manually to account for Tbx21 with ChIP peaks outside of the set boundaries (10 kb flanking the transcribed region).
E. The clustered heat map of Fos12 and core TF occupancy reveals that the binding domain of Fos12 largely overlaps with that of BATF.

FIG. 7. Model for Th17 TF functions during lineage specification.

Functions of the core TFs and a selection of newly-identified TFs in regulating expression of general T-helper cell- and Th17 cell-associated genes. BATF/IRF4 complexes, transcriptionally induced following TCR signaling, mutually activate the expression of a large set of target genes, together with STAT3. RORγt drives expression of a small subset of key Th17 genes and modulates the expression of genes activated by initiator TFs, BATF/IRF4/STAT3. Fos12 restricts the expression of genes required for alternate CD4⁺ differentiation programs. c-Maf functions as a general repressor. Regulatory hubs include loci that receive a high level of input from Th17 TFs and are enriched for genes that are critical for Th17 differentiation and function.

FIG. 8/Table 3. List of Exemplary TRNs, including NCBI Gene ID numbers for mouse and human TRN protein nucleic and amino acid sequences.

FIG. S1. Genome-wide co-occupancy of Th17 lineage TFs.

A. In vitro model system for Th17 cell differentiation from naïve CD4⁺ T cell precursors and schematic for experimental protocol. Th0 cultures provide control cells that receive TCR activation in the absence of exogenous polarizing cytokines (IL-6+ TGFβ).
B. Western blot analysis of Th17 polarization time series starting from FACS-purified naïve CD4 T cells (time=0).
C. Recovery of cognate consensus motifs from TF-ChIP-Seq.
D. High degree of co-occupancy among Th17 lineage TFs. ChIP-seq binding tracks are displayed for core TFs, CTCF, and p300 at selected Th17 loci in both non-polarized Th0 and Th17 conditions. Visualized using the Integrative Genomics Viewer (IGV; Broad Institute).
E. High-order pCRMs are not correlated with proximity to TSS. Bar chart of proportion of proximal versus distal pCRMs with respect to increasing order of occupancy. See related FIG. 1.

FIG. S2. Cooperative occupancy by BATF and IRF4.

A. Genome-wide interdependence of IRF4 and BATF co-occupancy in Th0 cells. Box plots displaying the fold change in ChIP-seq reads for IRF4 in Batf wild-type (wt) versus knockout (ko) and for BATF in Irf4 (wt/ko) for 48 h Th0 cultured cells. Differences in ChIP-seq reads are assayed within relevant pCRM regions. Three sub-types of pCRMs were interrogated: BATF or IRF4 alone; BATF and IRF4 alone; and BATF, IRF4, plus additional TFs (+) as indicated by color-coding. Displayed is the data distribution: median (line), 25^{th} to 75^{th} percentile (box)+/−1.5 Interquartile range (whiskers). To compute fold change in ChIP values, reads localized to a given pCRM were normalized by library size (i.e. reads per million; RPM) prior to calculations.
B. Interdependent binding of IRF4 and BATF at selected loci in Th17 cells.
See related FIG. 2.

FIG. S3. Genome-wide requirement for Th17 TFs for accessibility and TF occupancy.

A. Spatial correlations between Th17 TFs within pCRMs occupied by all five TFs. Bar chart plots the occurrence with which the summit of a given TFs occupies relative position 1 through 5 when the summits of all five are ordered from 5′ to 3′.
B. IRF4 and BATF regulate chromatin accessibility at TCR-induced cis regions that are co-occupied by Th17 TFs. FAIRE signal at Th17 STF+p300 pCRMs was compared between WT and Irf4^{-/-} and Batf^{-/-} Th0 and Th17 polarized T cells. pCRMs were divided according to their accessibility status in naïve CD4⁺ T cells: constitutive pCRMs are accessible in naïve cells (2,930 regions, left panel), while induced pCRMs are not (1,575 regions, right). Biological replicate samples were averaged, and normalized FAIRE reads were aligned around the median summit position of overlapping TF binding peaks, +/−2,000 bp.
C. Limited requirement for RORγt for p300 occupancy as compared to IRF4, BATF, and STAT3. Differential occupancy of p300 in TF wild-type versus deficient 48 h Th17 polarization cultures is displayed as scatter plots of fold change versus significance. Various pCRM subtypes are compared as indicated in the figure. Percentage of pCRMs with differential ChIP are indicated in plot.
D. Limited requirement for RORγt for IRF4 and STAT3 occupancy and for presence of H3K4me2 and H3K4me3 modifications. Scatter plots as in (C).

FIG. S4. Relationship between core TF regulation and expression of target genes in the Th17 network.

A. Heat map summarizing genome-wide regulatory inputs for the core TF network displayed in FIG. 3A. Orange represents repression and blue represents activation. Rows are target genes.
B. Heat map of activation and repression inputs for highly regulated genes (4 or 5 inputs) by core TFs. Orange represents repression and blue represents activation. View is limited to genes with KC scores>1.5 for a given TF-target regulatory interaction. Also displayed is the fold change (FC) in expression observed in Th17 relative to Th0 cells.
C. Effect of individual core TFs on target gene transcription. Positive and negative regulation by STAT3, IRF4, and BATF is well correlated with expression changes associated with Th17 differentiation. In contrast, the regulatory effect of RORγt is consistent with a modulatory role. Box plots display the fold change in expression of both TF activation (green) and repression (red) targets for Th17 relative to Th0 culture conditions. Displayed is the data distribution: median (line), 25^{th} to 75^{th} percentile (box)+/−1.5 Interquartile range (whiskers). Genes co-regulated by either 4 or 5 of the TFs are considered in this analysis.
See related FIG. 3.

FIG. S5. aucROC performance and comparison of two meta-analysis strategies: rank-based and Fisher's method

A. Recovery of validated biologically relevant Th17 targets based on integration of regulatory models from multiple functionally relevant TFs and of multiple data types. The graph summarizes area under curve (auc) of receiver operator curve (ROC) plot results indicating the degree to which 74 literature-based Th17-relevant genes are enriched as top network predictions under different data combinations. Individual TFs (scatter plot) versus combined TFs (bar plots) are compared for each combination of data types. As a reference, differential expression in Th17 vs. Th0 (dotted line) and random performance (dashed line; based on 200 simulations) are provided.
B. The KCRI network selectively recovers genes linked to SNPs that are associated with Th17-implicated inflammatory disease in GWAS studies. Gray bars and scatter plots in each column correspond to the recovery of SNP-associated disease-relevant targets as top predictions within the ranked list of the KCRI network scores, using the aucROC analysis. Gene lists of disease-associated SNPs were compiled from the National Human Genome Research GWAS Catalog.

FIG. S6. Gain and loss of function screens identify regulators of Th17 specification.

A. Overexpression screen of network TF candidates as putative Th17 subset regulators. Bar charts show the percent of IL-17A-producing or IFNγ-producing cells relative to the control empty vector after transduction of retroviruses encoding candidate factors and Th17 polarization for 48 h, or Th1 polarization for 5 days, respectively. Results are mean±s.e.m. for four biological replicates, each conducted in duplicate. Significance at *P<0.05 and **P<0.01 by T-test.
B. Representative flow cytometric analysis for IL-17A and Foxp3 expression in Th17-polarized cultures transduced with the indicated cDNAs in a retroviral vector also encoding the Thy1.1 reporter. Cells were gated for Thy1.1 expression to analyze proportions that were IL-17A⁺.
C. Knock-down efficiency of target mRNAs in the siRNA screen. Data represent reads per kilobase million (RPKM) expression values for the target TF in siRNA knock-down Th17 cultures relative to a non-targeting control. Analysis is at 24 h post Th17 differentiation.
D. Western-blot analysis of RORγt protein levels for Rorc knock-down at indicated times post initiation of Th17 polarization.
E. JMJD3 regulates the expression of many RORγt and STAT3 targets in Th17 cells. Network representation is as in FIG. 3. Due to space constraints, the display is limited to genes that have differential expression in Th17 relative to Th0 cells (z-score>2.5, <−2.5 based on statistical analysis of microarray for 8 independent experiments).
See related FIG. 5.

FIG. S7. Fos12 restricts the plasticity of Th17 subset cells.

A. Dysregulated cytokine production in the absence of Fos12. Fos12 wild-type and deficient naïve CD4⁺ T cells were polarized under Th1, Th2, and Th17 conditions for 6 days. Flow cytometric analysis was then performed for IL-17A, IL-4, and IFNγ.
B. Fos12 restricts IFNγ production among IL-17A-producing cells. Fos12 wild-type and deficient naïve CD4⁺ T cells were polarized under Th17 conditions (20 ng/mL IL-6 and 0.3 ng/mL TGFβ+blocking antibodies for IFNγ and IL-4) for three days. Thereafter, the media was replaced with cytokines for either (a) Th17-; (b) Th1-(long/mL IL-12); or (c) Th2-(2 ng/mL IL-4) promoting conditions for an additional three days prior to analysis.
C. De novo motif analysis of high confidence binding regions for BATF and Fos12 ChIP-seq experiments of Th17 cells showing that the AP-1 consensus motif is recovered in both instances.
D. Regulation of core Th17 TFs by Fos12. Edges represent integration of data from ChIP-seq and KO RNA-seq differential expression; line weight is relative to network score; FDR<5%. Nodes are colored to indicate the differential expression in Th17 relative to Th0 (blue=upregulated, orange=downregulated in Th17 cells).
See related FIG. 6.

FIG. S8/Table S1. Literature curated validation list for genes with critical influence for Th17 development or function. The list of known Th17-relevant genes used for computational validations is provided, including the Pubmed ID (PMID) for the supporting literature. See related FIG. 4.

FIG. S9/Table S2. Enrichment scores for identification of novel TFs and regulators. Candidate genes used in various biological screens (gain- and loss-of function) are highlighted in green, purple, and blue depending on the criteria used for their selection. Positive controls for TF recovery (STAT3, BATF, IRF4, Maf, and RORC) are highlighted in yellow. See related FIG. 5.

FIG. S10/Table S3. List of experimental libraries for ChIP-seq and RNA-seq.

FIG. S11/Table S4. TF Summed scores for KC and KCRI networks, including the top 500 candidate genes (genes encoding TRN proteins).

### DETAILED DESCRIPTION

Th17 cells exert critical functions in immune defense at mucosal barriers and are implicated as contributors to multiple autoimmune diseases (Korn et al., 2009). Since the discovery of Th17 cells, multiple TFs involved in the production of IL-17A and in inflammation were described, but little was known of how they collaborate in the global transcriptional program governing Th17 specification and function. Here we aimed to accurately define how Th17 TFs integrate functionally to execute this program, and created a useful network model that can be exploited to uncover novel lineage regulators, effectors, and potential therapeutic targets (FIG. 7). To achieve this, we used a culture system for Th17 differentiation to build a transcriptional network model based on combinations of datasets and analytical approaches, and determined its performance using both computational and experimental validations, testing the role of predicted regulators in vitro and in a disease model of inflammation. Our combined computational and experimental approach allowed for iteration between the generation of a data-integrative network and follow-up investigation of individual genes, and, hence, for continuous refinement of the network (FIG. 4A). This work provides a clear experimental design and analysis framework that can be adopted for other cell lineages in the immune system and elsewhere.

Dynamics of TF Function in the Specification of CD4⁺ T Cells:

Transcription initiation at sites occluded by nucleosomes and high-order chromatin structure requires mechanisms for making specific regions accessible to appropriate regulators (Zaret and Carroll, 2011). In TCR-activated CD4⁺ T cells, BATF and IRF4 bind cooperatively to sites throughout the genome. In the presence of Th17-polarizing cytokines, STAT3, c-Maf, and RORγt are recruited to many of the same sites. Chromatin accessibility analysis suggests that BATF/IRF4 complexes pioneer the access of other TFs that further specify functional subsets. Indeed, BATF and IRF4 have critical roles in multiple Th cells (Brustle et al., 2007; Ise et al., 2011; Rengarajan et al., 2002; Schraml et al., 2009). As these TFs are up-regulated in Th0 cells, it is interesting to speculate that pioneering function provides the T cell with plasticity to differentiate in multiple directions, depending on the cytokine environment. Thus, while TGF-β and STAT3-activating signals would recruit STAT3/RORγt to a subset of BATF/IRF4 binding sites, Th1 or Th2 signals may recruit STAT1/T-bet or STAT6/GATA-3 to others. It will be of interest to compare the global distribution of BATF, IRF4, and lineage-specifying TFs in Th1 and Th2 cells.

Fos12 is a negative regulator of IL-17A. Thus, the finding that Fos12-deficient mice had a reduced inflammatory response in the EAE model was unexpected. This may reflect the requirement for Fos12 for expression of key loci supporting Th17 cell maintenance. The result may also be explained by derepression of Foxp3 in inflammatory T cells producing IL-17A, IFNγ, and GM-CSF, which may be mediated, in part, by reduced expression of Hif1α—a Foxp3 inhibitor—in Fos12-deficient T cells (Dang et al., 2011). Foxp3⁺ T helper cells that produce effector cytokines have been described in humans and have been shown to have regulatory activity (Voo et al., 2009). Hence, reduced disease scores in Fos12 deficiency may be due to increased activity of Treg-like cells infiltrating the CNS. Fos12-deficient T cells also derepress T-bet and IFNγ, suggesting that Fos12 serves as a brake, binding to sites otherwise occupied by BATF and IRF4 to prevent expression. Indeed, Fos12 occupancy overlaps with that of BATF and IRF4 in Th0 and Th17 cells, where it likely competes for AP-1 sites (FIG. 7). Thus, Fos12 is a highly integrated regulator of T helper cell lineage identity, functioning to limit plasticity of Th17 cells by repressing Th1 and Treg transcriptional programs, potentially by balancing the activity of BATF/IRF4 at key loci. Our analysis also highlights that regulation of a single cytokine, i.e. IL-17A, does not reflect broad functions of the controlling TFs. A global perspective in the context of a multi-TF causal regulatory network aids in deciphering the role of individual factors.

RORγt has been described as a “master regulator” for the Th17 program, yet it has a surprisingly small regulatory footprint. RORγt deficiency had limited effects on p300 recruitment and H3K4 methylation, suggesting that it lacks a major role in remodeling its regulated loci. However, a handful of loci were highly dependent on RORγt for these early inductive events (FIG. 7); what distinguishes this selectivity remains to be uncovered. This focal mode of regulation, coupled with a generalized program upon which RORγt functions to tune expression, is consistent with the plasticity of Th17 cells, suggesting that expression and chromatin state at key Th17 loci might be amenable to rapid change depending on cytokine environment. The lack of stabilizing positive feedback of RORγt to initiators may permit such T-helper program switching. Moreover, while RORγt attenuates the expression of regulators of alternative Th subsets (il4ra, il12rb, Tbx21), these loci are nevertheless expressed. Thus, RORγt is not a prototypical “master” regulator that functions to “lock-in” lineage programs. This renders RORγt an exceptional drug target, as therapeutic intervention would not be expected to perturb the generic regulatory programs shared by other cell types.

A Highly Predictive Th17 Cell Network Model:

The iterative approach applied here was successful in uncovering important aspects of Th17 biology, generating a model that captures most of the previously identified Th17-relevant genes among the top candidates and predicting many more with equal confidence. Among these, several TFs and chromatin modifiers were shown to affect Th17 differentiation or the expression of immune-modulatory genes, including Bcl11b, Etv6, and Jmjd3. Although we focused on the top predicted TF Fos12, we expect that many more candidates will be pertinent to Th17 biology. We anticipate the network model presented herein to be a highly useful tool for exploration and for generation of new hypotheses.

Although the Th17 network largely models in vitro differentiation, it is nonetheless likely to be relevant for in vivo Th17 cell functions. Indeed, Fos/2-mutant T cells were compromised in effector function in an autoimmunity model and similar phenotypes were reported in mice deficient for many top scoring network genes. Moreover, the network is selectively enriched for genes with orthologs that harbor SNPs associated with human inflammatory diseases linked to Th17 cell-mediated pathology, such as Crohn's disease and psoriasis. GWAS studies, while facilitating the identification of genes involved in complex diseases involving multiple cell types, are often difficult to translate into biological hypotheses amenable to investigation. However, our analysis identified several GWAS-implicated genes as candidate Th17-specific mediators of pathogenesis (e.g. PTPN22, LIF, KLF6) and may be used to implicate Th17 cells in the etiology of particular conditions. Deconvoluting GWAS data by leveraging the information from accurate and comprehensive transcriptional regulatory networks to provide cellular context, reveal functional epistasis, and prioritize genes of potential medical importance will likely prove to be a powerful approach in uncovering disease mechanisms and developing new diagnostic and therapeutic tools (Califano et al., 2012). Taken together, this body of work is an excellent example of how the power of systems biology can be harnessed to answer a specific large-scale biological question, thus providing a validated paradigm for similar undertakings.

Table 1 presents a list of exemplary TRN proteins selected from the larger list of TRN proteins presented in Table S4. See also FIG. 8 (Table 3) for information pertaining to NCBI Gene ID numbers for access to nucleic and amino acid sequences corresponding to the exemplary TRN proteins listed in Tables 1 and 2.

TABLE 1

```
  1 CPD
  2 SLCO3A1
  3 GPR68
  4 SMOX
  5 ITGA3
  6 FRMD4B
  7 CCDC63
  8 INPP5B
  9 TNK2
10 TSPAN5
11 WDR1
12 LPXN
13 WDFY2
14 ABTB2
15 CYTH4
16 PMEPA1
17 ADAM19
18 RNF19B
19 TRERF1
20 EGLN3
21 B4GALNT4
22 2010002N04RIK
23 CD5L
24 ATP6V0A1
25 PAQR8
26 PLEKHF1
27 FES
28 NCS1
29 MGLL
30 CYTH1
31 CYSLTR1
32 1190002N15RIK
33 VWA1
34 ATRNL1
35 B230312A22RIK
36 MPP2
37 SLC4A11
38 TMEM176B
39 TMEM176A
40 CYFIP1
41 S1PR2
42 CAPG
43 TGFBR3
44 KCNA3
45 DPY19L3
46 TMEM184B
47 PREX1
48 TNFRSF25
49 6430527G18RIK
50 ESPN
```

Table 2 presents a more particular list of exemplary TRN proteins selected from the larger list of TRN proteins presented in Table S4.

TABLE 2

```
  1 CPD
  2 SLCO3A1
  3 GPR68
  4 SMOX
  5 ITGA3
  6 FRMD4B
  7 CCDC63
  8 INPP5B
  9 TNK2
10 TSPAN5
11 LPXN
12 CYSLTR1
13 2010002N04RIK
14 PMEPA1
15 SLC4A11
16 FES
```

### Screening Assays

Th17 cells have critical roles in mucosal defense and are major contributors to inflammatory disease. Their differentiation requires the nuclear hormone receptor RORγt working with multiple other essential transcription factors (TFs). As described herein, the present inventors have used an iterative systems approach, combining genome-wide TF occupancy, expression profiling of TF mutants, and expression time series to delineate the Th17 global transcriptional regulatory network. We find that cooperatively-bound BATF and IRF4 contribute to initial chromatin accessibility, and with STAT3 initiate a transcriptional program that is then globally tuned by the lineage-specifying TF RORγt, which plays a focal deterministic role at key loci. Integration of multiple datasets allowed inference of an accurate predictive model that we computationally and experimentally validated, identifying multiple new Th17 regulators, including Fos12, a key determinant of cellular plasticity. This interconnected network can be used to investigate new therapeutic approaches to manipulate Th17 functions in the setting of inflammatory disease.

Screening assays to identify and characterize modulators of Th17 cell function and/or specification as described herein may be performed using non-cell based assays and/or cell based assays. Such assays may be performed using full length Th17 regulatory network (TRN) proteins or functional fragments thereof.

Full length TRN proteins or functional fragments thereof may be labeled with a variety of protein tags, including detectable moieties that confer the ability to detect interaction/binding of labeled proteins to which they are attached and binding moieties, such as His Tags and GST tags that confer particular binding properties to proteins to which they are attached. Detectable labels include, for example, fluorescein, rhodamine, Texas Red, cyan fluorescent protein (CFP), yellow fluorescent protein (YFP), and green fluorescent protein (GFP) for visualization/detection. Full length TRN proteins or functional fragments thereof may either be in solution or bound to a solid surface depending on the assay.

### Non-Cell Based Assays

A variety of non-cell based assays have been described that may be utilized to identify modulators of Th17 cell specification, including affinity-based methods (e.g., affinity chromatography or panning), competitive inhibition assays (e.g., ELISAs), BIAcore assays, assays involving Covaspheres, and various visualization techniques, such as fluorescence resonance energy transfer (FRET). Each of these assays is well known in the art and may be performed in keeping with standard procedures.

In brief, BIAcore technology is based on surface plasmon resonance (SPR), an optical phenomenon that enables detection of unlabeled interactants in real time. SPR-based biosensors are used to determine the active concentration and assess molecular interactions, both with respect to affinity and chemical kinetics. A basic interaction experiment involves immobilizing one molecule of a binding pair on the sensor chip surface (“ligand”) and injecting a series of concentrations of its partner (“analyte”) across the surface. Changes in the index of refraction at the surface where the binding interaction occurs are detected by the hardware and recorded as RU (resonance units) in the control software. Curves are generated from the RU trace and are evaluated by fitting algorithms which compare the raw data to well-defined binding models. These fits are used to determine a variety of thermodynamic constants, including the apparent affinity of the binding interaction. Additional details pertaining to such BIAcore assays are known in the art and have been applied to a variety of cellular adhesion molecules. See, for example, Jin et al. (2008, Exp Biol Med 233:849-859); Chen et al. (2007, Proc Natl Acad Sci 104:13901-6); Sivakumar et al. (2007, J Biol Chem 282:7312-9); Catimel et al. (2005, J Proteome Res 4:1646-1656); and Syed et al. (2002, Biochem J 362:317-327), the entire content of each of which is incorporated herein by reference.

In one embodiment, direct interaction of a full length TRN protein or a functional fragment thereof may be assessed in a BIAcore assay. In a particular embodiment thereof, the full length TRN protein or a functional fragment thereof, for example, is expressed as a GST fusion protein and immobilized on a BIAcore chip (the “ligand”) and a protein known to bind/interact directly with the indicated ligand (analyte) or a fragment or peptide thereof (the “analyte”) is flowed over the chip to measure binding kinetics. Based on the kinetics, dissociation constants can be calculated. Once the basic parameters of binding interactions are established for the BIAcore assay, potential modulators of ligand/analyte interaction/binding can be added to the unbound molecules in advance of flow over the chip or during the flow over the chip step. In a particular embodiment thereof, if the presence of a modulator in the solution of molecules before or during the flow over the chip step reduces or inhibits binding of the analytes to the ligands, the modulator is identified as an inhibitor of the ligand/analyte interaction/binding and thus, is a potential inhibitor of Th17 cell specification.

In another embodiment, a first population comprising full length TRN protein or a functional fragment thereof (ligand) is covalently bound to a solid surface, such as a Red or Green MX Covasphere, and a second population comprising a protein known to bind/interact directly with the indicated ligand (analyte) or a fragment or peptide thereof (analyte) is bound to a solid surface such as a Petri dish. Homophilic interaction of Covasphere-bound ligand and Petri dish-bound analyte can be detected by visualization of fluorescent Covaspheres bound to the Petri dish. Suitable controls would be performed to ensure that binding of Covaspheres is due to bona fide ligand/analyte interaction, as opposed to non-specific interactions. The activity of putative modulators of ligand/analyte binding could be evaluated by performing the above assay in the presence or absence of putative modulators. A putative modulator would be identified as an inhibitor, for example, if ligand/analyte binding (as measured by Covasphere binding to the Petri dish) is reduced in its presence. Similar methods have been used to delineate domains of various cell adhesion molecules, including L1 (Zhao et al. 1995, J Biol Chem 270:29413-29421; the entire content of which is incorporated herein by reference).

In yet another embodiment, protein interactions and modulation thereof can be examined using FRET. In brief, FRET is based on energy transfer between two chromophores. A donor chromophore, initially in its electronic excited state, may transfer energy to an acceptor chromophore through nonradiative dipole-dipole coupling. The efficiency of this energy transfer is inversely proportional to the sixth power of the distance between donor and acceptor, thus making FRET extremely sensitive to small distances. Accordingly, FRET can be used to measure the distance between two fluorophores. When adapted to analyses of protein-protein interactions, FRET can be used advantageously to determine if two proteins that are differentially labeled (i.e., wherein each is labeled with a different fluorophore) bind to each other since the FRET readout will differ depending on whether the differentially labeled proteins are bound to each other or not. Exemplary pairs of fluorophores for use in FRET based analyses of protein-protein interactions comprise cyan fluorescent protein (CFP) and yellow fluorescent protein (YFP). Both of which are color variants of green fluorescent protein (GFP). Labeling with organic fluorescent dyes requires purification, chemical modification, and intracellular injection of a host protein. Alternatively, GFP variants can be attached to a host protein by genetic engineering. Bioluminescence Resonance Energy Transfer (BRET) provides an alternative system wherein a bioluminescent luciferase (typically that of Renilla reniformis), rather than CFP is used to produce an initial photon emission compatible with YFP.

Accordingly, in an application of FRET, a first population comprising full length TRN protein or a functional fragment thereof (ligand), for example, is labeled with CFP and a second population comprising a protein known to bind/interact directly with the indicated ligand (analyte) or a fragment or peptide thereof is labeled with YFP and the two populations are brought into contact. Detection of binding/interaction between the differentially labeled first and second populations can be achieved by measuring fluorescence emissions that change based on proximity of the different labels. As described herein above with respect to other screening assays, the activity of putative modulators of ligand/analyte binding could be evaluated by performing the above assay in the presence or absence of putative modulators. A putative modulator would be identified as an inhibitor, for example, if ligand/analyte binding (as measured by a change in fluorescence emissions) was reduced in its presence.

### Cell Based Assays

Cell based assays are presented herein as an alternative primary screening assay or as a secondary screening assay to validate the activity of modulators identified in non-cell based assays. Cell based assays may be performed in any cell line that is capable of responding to TRN signaling. Such cells may endogenously express cellular proteins that confer responsiveness to TRN signaling or a particular component thereof or may be engineered to respond to TRN signaling or a particular component thereof. Accordingly, such cell based assays may utilize prokaryotic or eukaryotic cells, including, without limitation, insect cell lines (such as, e.g., S2 cells) and mammalian cells and cell lines. An exemplary cell based assay involves, for example, mammalian T cells. Such cells may originate from any mammal, including, without limitation, mice, rats, monkeys, and humans. In a particular embodiment thereof, the cell population used in a cell based assay is a population of activated CD4+ T cells subjected to Th17 polarizing conditions such as that described in the Examples presented herein.

Further to the discovery of a regulatory network for Th17 cell specifications, the present inventors have developed cell based screening assays to identify agents/molecules capable of modulating TRN interaction/binding with other proteins (ligands) and thereby modulating TRN function and signaling. Such screening assays provide systems for identifying novel therapeutic agents and developing strategies to modulate immune responses qualitatively to, for example, reduce or abrogate Th17 cell mediated immune responses in patients in need thereof.

In a particular embodiment, cell based screening assays directed to identifying agents/molecules capable of inhibiting/blocking TRN signaling (see, e.g., FIGS. 5A-D and S6) are described. Such screening assays may, for example, be cell based assays that utilize cells that express endogenously express cellular proteins that confer responsiveness to TRN signaling or a particular component thereof or may be engineered to respond to TRN signaling or a particular component thereof. As described herein above with respect to non-cell based assays, cell based assays that are engineered to respond to TRN signaling or a particular component thereof may comprise TRN proteins or fragments thereof that are epressed with or without tags, such as, for example a Histidine (His) tag or the like.

In a particular embodiment of a cell-based assay, a population of activated CD4+ T cells subjected to Th17 polarizing conditions such as that described in the Examples set forth herein; (see also FIGS. 5A-D and S6 and descriptions thereof) is utilized and incubated in the presence or absence of a potential candidate agent or plurality of candidate agents to determine if the presence of the candidate agent/s modulates TRN signaling. Changes in transcription factor (TF)-dependent loci, including a variety of helper T cell effector genes (il17a, Il17f, Il22, Illr1, Il23r, Il10, Il24, Il9, Ccl20) and lineage-specializing genes (Rorc, Gata3, Foxp3, Tbx21, Il4, Ifng) can be used as readouts for assessing modulation of TRN signaling. See, e.g., FIG. 5C and descriptions thereof. Changes in a broad set of helper T cell-modulatory genes also implicate Bcl11b in TRN signaling. Alterations in Jmjd3 activity as reflected in activation of multiple Th17-expressed cytokines also offers Jmjd3 and its targets as indicator genes or readouts for assessing modulation of TRN signaling in the presence of a potential modulatory agent.

Further to the above, the siRNAs described herein are exemplary inhibitors of TRN proteins described herein.

Reagents are commercially available to investigate further the role of Jmjd3 in Th17 specification. Recombinant human JMJD3/KDM6B histone demethylase (amino acids 1043-end; Genbank Accession No. NM_{—}001080424) with a C-terminal FLAG-tag is available, for example, from BPS Bioscience (Cat #50115). This fusion protein was expressed in Sf9 cells via a baculovirus expression system. A colorimetric assay designed to quantitate JMJD3/UTS demethylase activity/inhibition is also available from Epigentek (Cat #3084; Epigenase™) Methods for screening to identify selective inhibitors of Jumonji C domain-containing histone demethylases have been described and selective inhibitors thereof have been characterized. See, for example, Luo et al. (2011 J American Chem Soc 133; 9451-9456), the entire content of which is incorporated herein by reference.

Further to the above, a specific inhibitor of JMJD3 (GSK-J1) has been developed, the chemical structure of which is shown below alone and bound in the catalytic pocket of human JMJD3.

The above is taken from Kruidenier et al. Nature 488:404-408 (2012), the entire content of which is incorporated herein by reference.

With respect to function, methylated lysine residues of histone 3 lysine 27 (H3K27me) contribute to regulation of gene activity by silencing through the polycomb-repressive complex (PRC1 or PRC2). This epigenetic mark can be demethylated by lysine demethylase (KDM) enzymes UTX and JmjD3 in humans, which play important roles in cellular differentiation, development and cancer. GSK-J1 is the first selective and potent histone demethylase inhibitor shown to have significant activity (IC₅₀ 60 nM for human JmjD3) in vitro and in cells using an ester derivative (GSK-J4: 1 μM<IC₅₀<10 μM; e.g. 9 μM in primary human macrophages). The pyridine region-isomer GSK-J2 displays significantly less on-target activity (IC₅₀>100 μM for human JmjD3) and thus can be used as control for target effects in vitro, and as ester derivative (GSK-J5) in cells.

Additional screening assays for assaying histone demethylase inhibitors are set forth in U.S. Patent Application No. 2012/0202875, the entire content of which is incorporated herein by reference.

Thus, in accordance with the results presented herein, GSK-J1 is set forth as an exemplary inhibitor of Th17 specification and inflammatory conditions and autoimmune diseases associated with Th17 mediated pathology.

Agents identified using the screening assays described herein or appreciated in light of the discoveries presented herein can be used in therapeutic applications directed to promoting or inhibiting Th17 cell mediated responses and, more particularly, to reduce or abrogate Th17 cell mediated immune responses in subjects in need thereof.

In accordance with the present invention there may be employed conventional molecular biology, microbiology, and recombinant DNA techniques within the skill of the art. Such techniques are explained fully in the literature. See, e.g., Sambrook et al, “Molecular Cloning: A Laboratory Manual” (1989); “Current Protocols in Molecular Biology” Volumes I-III [Ausubel, R. M., ed. (1994)]; “Cell Biology: A Laboratory Handbook” Volumes I-III [J. E. Celis, ed. (1994))]; “Current Protocols in Immunology” Volumes I-III [Coligan, J. E., ed. (1994)]; “Oligonucleotide Synthesis” (M. J. Gait ed. 1984); “Nucleic Acid Hybridization” [B. D. Hames & S. J. Higgins eds. (1985)]; “Transcription And Translation” [B. D. Hames & S. J. Higgins, eds. (1984)]; “Animal Cell Culture” [R. I. Freshney, ed. (1986)]; “Immobilized Cells And Enzymes” [IRL Press, (1986)]; B. Perbal, “A Practical Guide To Molecular Cloning” (1984).

Therefore, if appearing herein, the following terms shall have the definitions set out below.

### A. TERMINOLOGY

The term “specific binding member” describes a member of a pair of molecules which have binding specificity for one another. The members of a specific binding pair may be naturally derived or wholly or partially synthetically produced. One member of the pair of molecules has an area on its surface, or a cavity, which specifically binds to and is therefore complementary to a particular spatial and polar organization of the other member of the pair of molecules. Thus the members of the pair have the property of binding specifically to each other. Examples of types of specific binding pairs are antigen-antibody, biotin-avidin, hormone-hormone receptor, receptor-ligand, enzyme-substrate. This application is concerned in part with antigen-antibody type reactions.

The term “antibody” describes an immunoglobulin whether natural or partly or wholly synthetically produced. The term also covers any polypeptide or protein having a binding domain which is, or is homologous to, an antibody binding domain. CDR grafted antibodies are also contemplated by this term. An “antibody” is any immunoglobulin, including antibodies and fragments thereof, that binds a specific epitope. The term encompasses polyclonal, monoclonal, and chimeric antibodies, the last mentioned described in further detail in U.S. Pat. Nos. 4,816,397 and 4,816,567. The term “antibody(ies)” includes a wild type immunoglobulin (Ig) molecule, generally comprising four full length polypeptide chains, two heavy (H) chains and two light (L) chains, or an equivalent Ig homologue thereof (e.g., a camelid nanobody, which comprises only a heavy chain); including full length functional mutants, variants, or derivatives thereof, which retain the essential epitope binding features of an Ig molecule, and including dual specific, bispecific, multispecific, and dual variable domain antibodies; Immunoglobulin molecules can be of any class (e.g., IgG, IgE, IgM, IgD, IgA, and IgY), or subclass (e.g., IgG1, IgG2, IgG3, IgG4, IgA1, and IgA2). Also included within the meaning of the term “antibody” is any “antibody fragment”.

An “antibody fragment” means a molecule comprising at least one polypeptide chain that is not full length, including (i) a Fab fragment, which is a monovalent fragment consisting of the variable light (VL), variable heavy (VH), constant light (CL) and constant heavy 1 (CH1) domains; (ii) a F(ab′)2 fragment, which is a bivalent fragment comprising two Fab fragments linked by a disulfide bridge at the hinge region; (iii) a heavy chain portion of an Fab (Fd) fragment, which consists of the VH and CH1 domains; (iv) a variable fragment (Fv) fragment, which consists of the VL and VH domains of a single arm of an antibody, (v) a domain antibody (dAb) fragment, which comprises a single variable domain (Ward, E. S. et al., Nature 341, 544-546 (1989)); (vi) a camelid antibody; (vii) an isolated complementarity determining region (CDR); (viii) a Single Chain Fv Fragment wherein a VH domain and a VL domain are linked by a peptide linker which allows the two domains to associate to form an antigen binding site (Bird et al, Science, 242, 423-426, 1988; Huston et al, PNAS USA, 85, 5879-5883, 1988); (ix) a diabody, which is a bivalent, bispecific antibody in which VH and VL domains are expressed on a single polypeptide chain, but using a linker that is too short to allow for pairing between the two domains on the same chain, thereby forcing the domains to pair with the complementarity domains of another chain and creating two antigen binding sites (WO94/13804; P. Holliger et al Proc. Natl. Acad. Sci. USA 90 6444-6448, (1993)); and (x) a linear antibody, which comprises a pair of tandem Fv segments (VH-CH1-VH-CH1) which, together with complementarity light chain polypeptides, form a pair of antigen binding regions; (xi) multivalent antibody fragments (scFv dimers, trimers and/or tetramers (Power and Hudson, J Immunol. Methods 242: 193-204 9 (2000)); and (xii) other non-full length portions of heavy and/or light chains, or mutants, variants, or derivatives thereof, alone or in any combination.

As antibodies can be modified in a number of ways, the term “antibody” should be construed as covering any specific binding member or substance having a binding domain with the required specificity. Thus, this term covers antibody fragments, derivatives, functional equivalents and homologues of antibodies, including any polypeptide comprising an immunoglobulin binding domain, whether natural or wholly or partially synthetic. Chimeric molecules comprising an immunoglobulin binding domain, or equivalent, fused to another polypeptide are therefore included. Cloning and expression of chimeric antibodies are described in EP-A-0120694 and EP-A-0125023 and U.S. Pat. Nos. 4,816,397 and 4,816,567.

An antibody or antigen-binding portion thereof may, furthermore, be part of a larger immunoadhesion molecule, formed by covalent or noncovalent association of the antibody or antibody portion with one or more other proteins or peptides. Examples of such immunoadhesion molecules include use of the streptavidin core region to make a tetrameric scFv molecule (Kipriyanov, S. M., et al. (1995) Human Antibodies and Hybridomas 6:93 101) and use of a cysteine residue, a marker peptide and a C-terminal polyhistidine tag to make bivalent and biotinylated scFv molecules (Kipriyanov, S. M., et al. (1994) Mol. Immunol. 31:1047 1058). Antibody portions, such as Fab and F(ab′)₂ fragments, can be prepared from whole antibodies using conventional techniques, such as papain or pepsin digestion, respectively, of whole antibodies. Moreover, antibodies, antibody portions and immunoadhesion molecules can be obtained using standard recombinant DNA techniques, as described herein.

Further to the above, antibodies may be xenogeneic, allogeneic, or syngeneic; or modified forms thereof, e.g. humanized, chimeric, etc. Preferably, antibodies of the invention bind specifically or substantially specifically to a TRN protein described herein. The term “humanized antibody”, as used herein, is intended to include antibodies made by a non-human cell having variable and constant regions which have been altered to more closely resemble antibodies made by a human cell. This may be achieved by altering the non-human antibody amino acid sequence to incorporate amino acids found in human germline immunoglobulin sequences. The humanized antibodies of the invention may include amino acid residues not encoded by human germline immunoglobulin sequences (e.g., mutations introduced by random or site-specific mutagenesis in vitro or by somatic mutation in vivo), for example in the CDRs. The term “humanized antibody”, as used herein, also includes antibodies in which CDR sequences derived from the germline of another mammalian species, such as a mouse, have been grafted onto human framework sequences.

An “antibody combining site” is that structural portion of an antibody molecule comprised of light chain or heavy and light chain variable and hypervariable regions that specifically binds antigen.

The phrase “antibody molecule” in its various grammatical forms as used herein contemplates both an intact immunoglobulin molecule and an immunologically active portion of an immunoglobulin molecule.

Exemplary antibody molecules are intact immunoglobulin molecules, substantially intact immunoglobulin molecules and those portions of an immunoglobulin molecule that contain the paratope, including those portions known in the art as Fab, Fab′, F(ab′)₂ and F(v), which portions are preferred for use in the therapeutic methods described herein.

Antibodies may also be bispecific, wherein one binding domain of the antibody is a specific binding member of the invention, and the other binding domain has a different specificity, e.g. to recruit an effector function or the like. Bispecific antibodies of the present invention include wherein one binding domain of the antibody is a specific binding member of the present invention, including a fragment thereof, and the other binding domain is a distinct antibody or fragment thereof, including that of a distinct anti-cancer or anti-tumor specific antibody. The other binding domain may be an antibody that recognizes or targets a particular cell type, as in a neural or glial cell-specific antibody. In the bispecific antibodies of the present invention the one binding domain of the antibody of the invention may be combined with other binding domains or molecules which recognize particular cell receptors and/or modulate cells in a particular fashion, as for instance an immune modulator (e.g., interleukin(s)), a growth modulator or cytokine (e.g. tumor necrosis factor (TNF) or a toxin (e.g., ricin) or anti-mitotic or apoptotic agent or factor.

The phrase “monoclonal antibody” in its various grammatical forms refers to an antibody having only one species of antibody combining site capable of immunoreacting with a particular antigen. A monoclonal antibody thus typically displays a single binding affinity for any antigen with which it immunoreacts. A monoclonal antibody may also contain an antibody molecule having a plurality of antibody combining sites, each immunospecific for a different antigen; e.g., a bispecific (chimeric) monoclonal antibody.

The term “antigen binding domain” describes the part of an antibody which comprises the area which specifically binds to and is complementary to part or all of an antigen. Where an antigen is large, an antibody may bind to a particular part of the antigen only, which part is termed an epitope. An antigen binding domain may be provided by one or more antibody variable domains. Preferably, an antigen binding domain comprises an antibody light chain variable region (VL) and an antibody heavy chain variable region (VH).

The term “specific” may be used to refer to the situation in which one member of a specific binding pair will not show any significant binding to molecules other than its specific binding partner(s). The term is also applicable where e.g. an antigen binding domain is specific for a particular epitope which is carried by a number of antigens, in which case the specific binding member carrying the antigen binding domain will be able to bind to the various antigens carrying the epitope.

The term “adjuvant” refers to a compound or mixture that enhances the immune response, particularly to an antigen. An adjuvant can serve as a tissue depot that slowly releases the antigen and also as a lymphoid system activator that non-specifically enhances the immune response (Hood et al., Immunology, Second Ed., 1984, Benjamin/Cummings: Menlo Park, Calif., p. 384). Often, a primary challenge with an antigen alone, in the absence of an adjuvant, will fail to elicit a humoral or cellular immune response. Previously known and utilized adjuvants include, but are not limited to, complete Freund's adjuvant, incomplete Freund's adjuvant, saponin, mineral gels such as aluminum hydroxide, surface active substances such as lysolecithin, pluronic polyols, polyanions, peptides, oil or hydrocarbon emulsions, keyhole limpet hemocyanins, dinitrophenol, and potentially useful human adjuvant such as BCG (Bacille Calmette-Guerin) and Corynebacterium parvum. Mineral salt adjuvants include but are not limited to: aluminum hydroxide, aluminum phosphate, calcium phosphate, zinc hydroxide and calcium hydroxide. Preferably, the adjuvant composition further comprises a lipid of fat emulsion comprising about 10% (by weight) vegetable oil and about 1-2% (by weight) phospholipids. Preferably, the adjuvant composition further optionally comprises an emulsion form having oily particles dispersed in a continuous aqueous phase, having an emulsion forming polyol in an amount of from about 0.2% (by weight) to about 49% (by weight), optionally a metabolizable oil in an emulsion-forming amount of up to 15% (by weight), and optionally a glycol ether-based surfactant in an emulsion-stabilizing amount of up to about 5% (by weight).

As used herein, the term “immunomodulator” refers to an agent which is able to modulate an immune response. An example of such modulation is an enhancement of cell activation or of antibody production.

The term “effective amount” of an immunomodulator refers to an amount of an immunomodulator sufficient to enhance a vaccine-induced immune response, be it cell-mediated, humoral or antibody-mediated. An effective amount of an immunomodulator, if injected, can be in the range of about 0.1-1,000 μg, preferably 1-900 μg, more preferably 5-500 μg, for a human subject, or in the range of about 0.01-10.0 μg/Kg body weight of the subject animal. This amount may vary to some degree depending on the mode of administration, but will be in the same general range. If more than one immunomodulator is used, each one may be present in these amounts or the total amount may fall within this range. An effective amount of an antigen may be an amount capable of eliciting a demonstrable immune response in the absence of an immunomodulator. For many antigens, this is in the range of about 5-100 μg for a human subject. The appropriate amount of antigen to be used is dependent on the specific antigen and is well known in the art.

The exact effective amount necessary will vary from subject to subject, depending on the species, age and general condition of the subject, the severity of the condition being treated, the mode of administration, etc. Thus, it is not possible to specify an exact effective amount. However, the appropriate effective amount may be determined by one of ordinary skill in the art using only routine experimentation or prior knowledge in the vaccine art.

An “immunological response” to a composition or vaccine comprised of an antigen is the development in the host of a cellular- and/or antibody-mediated immune response to the composition or vaccine of interest. Usually, such a response consists of the subject producing antibodies, B cells, helper T cells, suppressor T cells, and/or cytotoxic T cells directed specifically to an antigen or antigens included in the composition or vaccine of interest.

The term “comprise” is generally used in the sense of include, that is to say permitting the presence of one or more features or components.

The term “consisting essentially of” refers to a product, particularly a peptide sequence, of a defined number of residues which is not covalently attached to a larger product. In the case of the peptide of the invention referred to above, those of skill in the art will appreciate that minor modifications to the N- or C-terminal of the peptide may however be contemplated, such as the chemical modification of the terminal to add a protecting group or the like, e.g. the amidation of the C-terminus.

The term “isolated” refers to the state in which specific binding members of the invention, or nucleic acid encoding such binding members will be, in accordance with the present invention. Members and nucleic acid will be free or substantially free of material with which they are naturally associated such as other polypeptides or nucleic acids with which they are found in their natural environment, or the environment in which they are prepared (e.g. cell culture) when such preparation is by recombinant DNA technology practised in vitro or in vivo. Members and nucleic acid may be formulated with diluents or adjuvants and still for practical purposes be isolated—for example the members will normally be mixed with gelatin or other carriers if used to coat microtiter plates for use in immunoassays, or will be mixed with pharmaceutically acceptable carriers or diluents when used in diagnosis or therapy.

As used herein, “pg” means picogram, “ng” means nanogram, “ug” or “μg” mean microgram, “mg” means milligram, “ul” or “μl” mean microliter, “ml” means milliliter, “l” means liter.

The amino acid residues described herein are preferred to be in the “L” isomeric form. However, residues in the “D” isomeric form can be substituted for any L-amino acid residue, as long as the desired functional property of immunoglobulin-binding is retained by the polypeptide. NH₂ refers to the free amino group present at the amino terminus of a polypeptide. COOH refers to the free carboxy group present at the carboxy terminus of a polypeptide. In keeping with standard polypeptide nomenclature, J. Biol. Chem., 243:3552-59 (1969), abbreviations for amino acid residues are shown in the following Table of Correspondence:

TABLE OF CORRESPONDENCE

| SYMBOL | | |
|---|---|---|
| 1-Letter | 3-Letter | AMINO ACID |
| Y | Tyr | tyrosine |
| G | Gly | glycine |
| F | Phe | phenylalanine |
| M | Met | methionine |
| A | Ala | alanine |
| S | Ser | serine |
| I | Ile | isoleucine |
| L | Leu | leucine |
| T | Thr | threonine |
| V | Val | valine |
| P | Pro | proline |
| K | Lys | lysine |
| H | His | histidine |
| Q | Gln | glutamine |
| E | Glu | glutamic acid |
| W | Trp | tryptophan |
| R | Arg | arginine |
| D | Asp | aspartic acid |
| N | Asn | asparagine |
| C | Cys | cysteine |

It should be noted that all amino-acid residue sequences are represented herein by formulae whose left and right orientation is in the conventional direction of amino-terminus to carboxy-terminus. Furthermore, it should be noted that a dash at the beginning or end of an amino acid residue sequence indicates a peptide bond to a further sequence of one or more amino-acid residues. The above Table is presented to correlate the three-letter and one-letter notations which may appear alternately herein.

A “replicon” is any genetic element (e.g., plasmid, chromosome, virus) that functions as an autonomous unit of DNA replication in vivo; i.e., capable of replication under its own control.

A “vector” is a replicon, such as plasmid, phage or cosmid, to which another DNA segment may be attached so as to bring about the replication of the attached segment.

A “DNA molecule” refers to the polymeric form of deoxyribonucleotides (adenine, guanine, thymine, or cytosine) in its either single stranded form, or a double-stranded helix. This term refers only to the primary and secondary structure of the molecule, and does not limit it to any particular tertiary forms. Thus, this term includes double-stranded DNA found, inter alia, in linear DNA molecules (e.g., restriction fragments), viruses, plasmids, and chromosomes. In discussing the structure of particular double-stranded DNA molecules, sequences may be described herein according to the normal convention of giving only the sequence in the 5′ to 3′ direction along the nontranscribed strand of DNA (i.e., the strand having a sequence homologous to the mRNA).

An “origin of replication” refers to those DNA sequences that participate in DNA synthesis.

A DNA “coding sequence” is a double-stranded DNA sequence which is transcribed and translated into a polypeptide in vivo when placed under the control of appropriate regulatory sequences. The boundaries of the coding sequence are determined by a start codon at the 5′ (amino) terminus and a translation stop codon at the 3′ (carboxyl) terminus. A coding sequence can include, but is not limited to, prokaryotic sequences, cDNA from eukaryotic mRNA, genomic DNA sequences from eukaryotic (e.g., mammalian) DNA, and even synthetic DNA sequences. A polyadenylation signal and transcription termination sequence will usually be located 3′ to the coding sequence.

Transcriptional and translational control sequences are DNA regulatory sequences, such as promoters, enhancers, polyadenylation signals, terminators, and the like, that provide for the expression of a coding sequence in a host cell.

A “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined by mapping with nuclease S1), as well as protein binding domains (consensus sequences) responsible for the binding of RNA polymerase. Eukaryotic promoters will often, but not always, contain “TATA” boxes and “CAT” boxes. Prokaryotic promoters contain Shine-Dalgarno sequences in addition to the −10 and −35 consensus sequences.

An “expression control sequence” is a DNA sequence that controls and regulates the transcription and translation of another DNA sequence. A coding sequence is “under the control” of transcriptional and translational control sequences in a cell when RNA polymerase transcribes the coding sequence into mRNA, which is then translated into the protein encoded by the coding sequence.

A “signal sequence” can be included before the coding sequence. This sequence encodes a signal peptide, N-terminal to the polypeptide, that communicates to the host cell to direct the polypeptide to the cell surface or secrete the polypeptide into the media, and this signal peptide is clipped off by the host cell before the protein leaves the cell. Signal sequences can be found associated with a variety of proteins native to prokaryotes and eukaryotes.

The term “oligonucleotide,” as used herein in referring to the probe of the present invention, is defined as a molecule comprised of two or more ribonucleotides, preferably more than three. Its exact size will depend upon many factors which, in turn, depend upon the ultimate function and use of the oligonucleotide.

The term “primer” as used herein refers to an oligonucleotide, whether occurring naturally as in a purified restriction digest or produced synthetically, which is capable of acting as a point of initiation of synthesis when placed under conditions in which synthesis of a primer extension product, which is complementary to a nucleic acid strand, is induced, i.e., in the presence of nucleotides and an inducing agent such as a DNA polymerase and at a suitable temperature and pH. The primer may be either single-stranded or double-stranded and must be sufficiently long to prime the synthesis of the desired extension product in the presence of the inducing agent. The exact length of the primer will depend upon many factors, including temperature, source of primer and use of the method. For example, for diagnostic applications, depending on the complexity of the target sequence, the oligonucleotide primer typically contains 15-25 or more nucleotides, although it may contain fewer nucleotides.

The primers herein are selected to be “substantially” complementary to different strands of a particular target DNA sequence. This means that the primers must be sufficiently complementary to hybridize with their respective strands. Therefore, the primer sequence need not reflect the exact sequence of the template. For example, a non-complementary nucleotide fragment may be attached to the 5′ end of the primer, with the remainder of the primer sequence being complementary to the strand. Alternatively, non-complementary bases or longer sequences can be interspersed into the primer, provided that the primer sequence has sufficient complementarity with the sequence of the strand to hybridize therewith and thereby form the template for the synthesis of the extension product.

As used herein, the terms “restriction endonucleases” and “restriction enzymes” refer to bacterial enzymes which cut double-stranded DNA at or near a specific nucleotide sequence.

A cell has been “transformed” by exogenous or heterologous DNA when such DNA has been introduced inside the cell. The transforming DNA may or may not be integrated (covalently linked) into chromosomal DNA making up the genome of the cell. In prokaryotes, yeast, and mammalian cells for example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones comprised of a population of daughter cells containing the transforming DNA. A “clone” is a population of cells derived from a single cell or common ancestor by mitosis. A “cell line” is a clone of a primary cell that is capable of stable growth in vitro for many generations.

Two DNA sequences are “substantially homologous” when at least about 75% (preferably at least about 80%, and most preferably at least about 90 or 95%) of the nucleotides match over the defined length of the DNA sequences. Sequences that are substantially homologous can be identified by comparing the sequences using standard software available in sequence data banks, or in a Southern hybridization experiment under, for example, stringent conditions as defined for that particular system. Defining appropriate hybridization conditions is within the skill of the art. See, e.g., Maniatis et al., supra; DNA Cloning, Vols. I & II, supra; Nucleic Acid Hybridization, supra.

It should be appreciated that also within the scope of the present invention are DNA sequences encoding TRN proteins or peptide sequences therein or comprising or consisting of sequences which are degenerate thereto. DNA sequences having the nucleic acid sequence encoding the peptides of the invention are contemplated, including degenerate sequences thereof encoding the same, or a conserved or substantially similar, amino acid sequence. By “degenerate to” is meant that a different three-letter codon is used to specify a particular amino acid. It is well known in the art that the following codons can be used interchangeably to code for each specific amino acid:

Phenylalanine (Phe or F)
UUU
or
UUC
Leucine (Leu or L)
UUA
or
UUG
or
CUU
or
CUC
or
CUA
or
CUG
Isoleucine (Ile or I)
AUU
or
AUC
or
AUA
Methionine (Met or M)
AUG
Valine (Val or V)
GUU
or
GUC of GUA
or
GUG
Serine (Ser or S)
UCU
or
UCC
or
UCA
or
UCG
or
AGU
or
AGC
Proline (Pro or P)
CCU
or
CCC
or
CCA
or
CCG
Threonine (Thr or T)
ACU
or
ACC
or
ACA
or
ACG
Alanine (Ala or A)
GCU
or
GCC
or
GCA
or
GCG
Tyrosine (Tyr or Y)
UAU
or
UAC
Histidine (His or H)
CAU
or
CAC
Glutamine (Gln or Q)
CAA
or
CAG
Asparagine (Asn or N)
AAU
or
AAC
Lysine (Lys or K)
AAA
or
AAG
Aspartic Acid (Asp or D)
GAU
or
GAC
Glutamic Acid (Glu or E)
GAA
or
GAG
Cysteine (Cys or C)
UGU
or
UGC
Arginine (Arg or R)
CGU
or
CGC
or
CGA
or
CGG
or
AGA
or
AGG
Glycine (Gly or G)
GGU
or
GGC
or
GGA
or
GGG
Tryptophan (Trp or W)
UGG
Termination codon
UAA (ochre)
or
UAG (amber)
or
UGA (opal)

It should be understood that the codons specified above are for RNA sequences. The corresponding codons for DNA have a T substituted for U.

Mutations can be made in the sequences encoding the protein or peptide sequences of the TRN proteins or peptides of the invention, such that a particular codon is changed to a codon which codes for a different amino acid. Such a mutation is generally made by making the fewest nucleotide changes possible. A substitution mutation of this sort can be made to change an amino acid in the resulting protein in a non-conservative manner (i.e., by changing the codon from an amino acid belonging to a grouping of amino acids having a particular size or characteristic to an amino acid belonging to another grouping) or in a conservative manner (i.e., by changing the codon from an amino acid belonging to a grouping of amino acids having a particular size or characteristic to an amino acid belonging to the same grouping). Such a conservative change generally leads to less change in the structure and function of the resulting protein. A non-conservative change is more likely to alter the structure, activity or function of the resulting protein. The present invention should be considered to include sequences containing conservative changes which do not significantly alter the activity or binding characteristics of the resulting protein.

The following is one example of various groupings of amino acids:

Amino Acids with Nonpolar R Groups

### Alanine, Valine, Leucine, Isoleucine, Proline, Phenylalanine, Tryptophan, Methionine

Amino Acids with Uncharged Polar R groups

### Glycine, Serine, Threonine, Cysteine, Tyrosine, Asparagine, Glutamine

Amino Acids with Charged Polar R Groups (Negatively Charged at pH 6.0)
Aspartic acid, Glutamic acid

### Basic Amino Acids (Positively Charged at pH 6.0)

### Lysine, Arginine, Histidine (at pH 6.0)

Another Grouping May be Those Amino Acids with Phenyl Groups:

### Phenylalanine, Tryptophan, Tyrosine

Another grouping may be according to molecular weight (i.e., size of R groups):

| | | | |
|---|---|---|---|
| Glycine | 75 | Alanine | 89 |
| Serine | 105 | Proline | 115 |
| Valine | 117 | Threonine | 119 |
| Cysteine | 121 | Leucine | 131 |
| Isoleucine | 131 | Asparagine | 132 |
| Aspartic acid | 133 | Glutamine | 146 |
| Lysine | 146 | Glutamic acid | 147 |
| Methionine | 149 | Histidine (at pH 6.0) | 155 |
| Phenylalanine | 165 | Arginine | 174 |
| Tyrosine | 181 | Tryptophan | 204 |

Particularly preferred substitutions are:
Lys for Arg and vice versa such that a positive charge may be maintained;
Glu for Asp and vice versa such that a negative charge may be maintained;
Ser for Thr such that a free —OH can be maintained; and
Gln for Asn such that a free NH₂ can be maintained.

Exemplary and preferred conservative amino acid substitutions include any of: glutamine (Q) for glutamic acid (E) and vice versa; leucine (L) for valine (V) and vice versa; serine (S) for threonine (T) and vice versa; isoleucine (I) for valine (V) and vice versa; lysine (K) for glutamine (Q) and vice versa; isoleucine (I) for methionine (M) and vice versa; serine (S) for asparagine (N) and vice versa; leucine (L) for methionine (M) and vice versa; lysine (L) for glutamic acid (E) and vice versa; alanine (A) for serine (S) and vice versa; tyrosine (Y) for phenylalanine (F) and vice versa; glutamic acid (E) for aspartic acid (D) and vice versa; leucine (L) for isoleucine (I) and vice versa; lysine (K) for arginine (R) and vice versa.

Amino acid substitutions may also be introduced to substitute an amino acid with a particularly preferable property. For example, a Cys may be introduced a potential site for disulfide bridges with another Cys. A His may be introduced as a particularly “catalytic” site (i.e., His can act as an acid or base and is the most common amino acid in biochemical catalysis). Pro may be introduced because of its particularly planar structure, which induces (3-turns in the protein's structure.

Two amino acid sequences are “substantially homologous” when at least about 70% of the amino acid residues, preferably at least about 80%, and most preferably at least about 90, 91, 92, 93, 94, 95, 96, 97, 98, or 99% of the amino acid residues are identical, or represent conservative substitutions.

A “heterologous” region of the DNA construct is an identifiable segment of DNA within a larger DNA molecule that is not found in association with the larger molecule in nature. Thus, when the heterologous region encodes a mammalian gene, the gene will usually be flanked by DNA that does not flank the mammalian genomic DNA in the genome of the source organism. Another example of a heterologous coding sequence is a construct where the coding sequence itself is not found in nature (e.g., a cDNA where the genomic coding sequence contains introns, or synthetic sequences having codons different than the native gene). Allelic variations or naturally-occurring mutational events do not give rise to a heterologous region of DNA as defined herein.

A DNA sequence is “operatively linked” to an expression control sequence when the expression control sequence controls and regulates the transcription and translation of that DNA sequence. The term “operatively linked” includes having an appropriate start signal (e.g., ATG) in front of the DNA sequence to be expressed and maintaining the correct reading frame to permit expression of the DNA sequence under the control of the expression control sequence and production of the desired product encoded by the DNA sequence. If a gene that one desires to insert into a recombinant DNA molecule does not contain an appropriate start signal, such a start signal can be inserted in front of the gene.

The term “standard hybridization conditions” refers to salt and temperature conditions substantially equivalent to 5×SSC and 65° C. for both hybridization and wash. However, one skilled in the art will appreciate that such “standard hybridization conditions” are dependent on particular conditions including the concentration of sodium and magnesium in the buffer, nucleotide sequence length and concentration, percent mismatch, percent formamide, and the like. Also important in the determination of “standard hybridization conditions” is whether the two sequences hybridizing are RNA-RNA, DNA-DNA or RNA-DNA. Such standard hybridization conditions are easily determined by one skilled in the art according to well known formulae, wherein hybridization is typically 10-20° C. below the predicted or determined Tₘ with washes of higher stringency, if desired.

As used herein, an “agent”, “candidate compound”, or “test compound” may be used to refer to, for example, nucleic acids (e.g., DNA and RNA), carbohydrates, lipids, proteins, antibodies, peptides, peptidomimetics, chemical compounds, small molecules and other drugs. In particular the term agent includes compounds such as test compounds or drug candidate compounds. The term “modulator agent” as used herein refers to an agent whose presence alters an interaction (e.g., a biochemical or physical interaction) relative to a control or inert agent. A modulator agent may, therefore, increase/enhance or decrease/reduce such an interaction relative to a control or inert agent. In a particular aspect, a modulator agent identified in a screening assay described herein inhibits TRN interactions with another protein/s, wherein the interaction promotes Th17 specification, and the modulator agent is, therefore, identified as an inhibitor of Th17 specification.

In a particular embodiment, the library of small compounds or agents can be purchased from a commercial vendor. Such libraries are known to those skilled in the art and are used routinely. An exemplary library of small molecules can be accessed at the worldwide web site provided by chembridge via screening libraries and more particularly, via diversity libraries (e.g., chembridge.com/screening libraries/diversity libraries).

The term ‘agonist’ refers to a ligand that stimulates the receptor to which the ligand binds in the broadest sense or stimulates a response that would be elicited on binding of a natural ligand to a binding site.

The term ‘assay’ means any process used to measure a specific property of a compound or agent. A ‘screening assay’ means a process used to characterize or select compounds based upon their activity from a collection of compounds.

“Preventing” or “prevention” refers to a reduction in risk of acquiring a disease or disorder.

The term ‘prophylaxis’ is related to and encompassed in the term ‘prevention’, and refers to a measure or procedure the purpose of which is to prevent, rather than to treat or cure a disease. Non-limiting examples of prophylactic measures may include the administration of vaccines; the administration of low molecular weight heparin to hospital patients at risk for thrombosis due, for example, to immobilization; and the administration of an anti-malarial agent such as chloroquine, in advance of a visit to a geographical region where malaria is endemic or the risk of contracting malaria is high.

“Therapeutically effective amount” means the amount of a compound that, when administered to a subject for treating a disease, is sufficient to effect such treatment for the disease. The “therapeutically effective amount” can vary depending on the compound, the disease and its severity, and the age, weight, etc., of the subject to be treated.

The term ‘treating’ or ‘treatment’ of any disease or infection refers, in one embodiment, to ameliorating the disease or infection (i.e., arresting the disease or growth of the infectious agent or bacteria or reducing the manifestation, extent or severity of at least one of the clinical symptoms thereof). In another embodiment ‘treating’ or ‘treatment’ refers to ameliorating at least one physical parameter, which may not be discernible by the subject. In yet another embodiment, ‘treating’ or ‘treatment’ refers to modulating the disease or infection, either physically, (e.g., stabilization of a discernible symptom), physiologically, (e.g., stabilization of a physical parameter), or both. In a further embodiment, ‘treating’ or ‘treatment’ relates to slowing the progression of a disease.

The phrase “pharmaceutically acceptable” refers to molecular entities and compositions that are physiologically tolerable and do not typically produce an allergic or similar untoward reaction, such as gastric upset, dizziness and the like, when administered to a human.

As used herein, the term “autologous” refers to organs, tissues, cells, or proteins isolated from a donor patient that are later re-introduced into the donor patient. Accordingly, the donor and recipient are the same patient in autologous transfers. The term “autologous T cells”, for example, refers to T cells that have been isolated from a subject and then administered to the same patient. Typically, and in accordance with the present methods, the isolated T cells may be stimulated in cell culture prior to administration to the patient.

### B. FURTHER ASPECTS OF THE DETAILED DESCRIPTION

The invention relates generally to methods and agents for modulation of immune responses, particularly those that involve Th17 cells. Prior to the discoveries detailed herein, there was no appreciation that certain of the proteins identified as components of the TRN played a role in Th17 specification. Fos12, for example, is a newly identified component of the Th17 specification program and thus, provides a novel target for modulation of Th17 specification. Indeed, results presented herein demonstrate that inhibition of Fos12 serves to inhibit Th17 activity and this, in turn, suggests that Fos12 inhibitors are promising agents for treating patients with inflammatory conditions or autoimmune disease. This present findings, therefore, provide insight into new screening assays and methods for using same to identify agents that modulate Th17 specification, agents identified thereby and therapeutic regimens utilizing these agents.

Accordingly, in a particular embodiment, methods and agents for inhibiting Th17 specification and thereby reducing inflammatory diseases linked to Th17 cell-mediated pathology, including Crohn's disease, ulcerative colitis, multiple sclerosis, rheumatoid arthritis, and psoriasis, are presented herein.

The present invention provides assays for screening and identifying agents, compounds or peptides to modulate Th17 specification and methods for reducing or inhibiting Th17 specification in the context of inflammatory disease. The methods, assays, and indicators described herein are based, in part, on the identification of novel targets (TRN proteins) and novel networks of TRN proteis that are important for Th17 specification. The methods, agents and assays of the invention can be implemented in therapeutic strategies directed to inhibition of Th17 specification.

The present invention also encompasses proteins described herein or agents identified using methods described herein which are covalently attached to or otherwise associated with other molecules or agents. These other molecules or agents include, but are not limited to, molecules (including antibodies or antibody fragments) with distinct recognition, targeting or binding characteristics, immune cell modulators, immune cell antigens, toxins, ligands, adjuvants, and chemotherapeutic agents.

Peptides and proteins of the invention may be labelled with a detectable or functional label. Detectable labels include, but are not limited to, radiolabels such as the isotopes ³H, ¹⁴C, ³²P, ³⁵S, ³⁶Cl, ⁵¹Cr, ⁵⁷Co, ⁵⁸Co, ⁵⁹Fe, ⁹⁰Y, ¹²¹I, ¹²⁴I, ¹²⁵I, ¹³¹I, ¹¹¹In, ¹¹⁷Lu, ²¹¹At, ¹⁹⁸Au, ⁶⁷Cu, ²²⁵Ac, ²¹³Bi, ⁹⁹Tc and ¹⁸⁶Re, which may be attached to antibodies of the invention using conventional chemistry known in the art of antibody imaging. Labels also include fluorescent labels (for example fluorescein, rhodamine, Texas Red) and labels used conventionally in the art for MRI-CT imaging. They also include enzyme labels such as horseradish peroxidase, β-glucoronidase, β-galactosidase, and urease. Labels further include chemical moieties such as biotin which may be detected via binding to a specific cognate detectable moiety, e.g. labelled avidin. Functional labels include substances which are designed to be targeted to the site of a tumor to cause destruction of tumor tissue. Such functional labels include cytotoxic drugs such as 5-fluorouracil or ricin and enzymes such as bacterial carboxypeptidase or nitroreductase, which are capable of converting prodrugs into active drugs at the site of a tumor.

Peptides of and of use in the present invention may include synthetic, recombinant or peptidomimetic entitites. The peptides may be monomers, polymers, multimers, dendrimers, concatamers of various forms known or contemplated in the art, and may be so modified or mutlimerized so as to improve activity, specificity or stability. For instance, and not by way of limitation, several strategies have been pursued in efforts to increase the effectiveness of antimicrobial peptides including dendrimers and altered amino acids (Tam et al (2002) Eur J Biochem 269 (3): 923-932; Janiszewska et al (2003) Bioorg Med Chem Lett 13 (21):3711-3713; Ghadiri et al. (2004) Nature 369(6478):301-304; DeGrado et al (2003) Protein Science 12(4):647-665; Tew et al. (2002) PNAS 99(8):5110-5114; Janiszewska et al (2003) Bioorg Med Chem Lett 13 (21): 3711-3713). U.S. Pat. No. 5,229,490 discloses a particular polymeric construction formed by the binding of multiple antigens to a dendritic core or backbone.

Protamines or polycationic amino acid peptides containing combinations of one or more recurring units of cationic amino acids, such as arginine (R), tryptophan (W), lysine (K), even synthetic polyarginine, polytryptophan, polylysine, have been shown to be capable of killing microbial cells. These peptides cross the plasma membrane to facilitate uptake of various biopolymers or small molecules (Mitchell D J et al (2002) J Peptide Res 56(5):318-325).

Conjugates or fusion proteins of the present invention, wherein TRN proteins or domains or fragments thereof or modulatory agents identified using screening methods as described herein are conjugated or attached to other molecules or agents further include, but are not limited to, binding members conjugated to a cell targeting agent or sequence, chemical ablation agent, toxin, immunomodulator, cytokine, cytotoxic agent, chemotherapeutic agent or drug.

Uptake and targeting of DCs, for example, can be achieved using a variety techniques known in the art, including coupling to antibodies targeting DC-specific surface molecules (Romani et al., 2010; the entire contents of which is incorporated herein in its entirety, including references cited therein); utilization of engineered Sindbis envelope that specifically target DC instead of VSV-G (Yang et al., 2008; the entire content of which is incorporated herein in its entirety); site of administration; blood infusion; or ex vivo culture of DC, treatment of ex vivo cultured DC to introduce the desired construct/s, and re-injection of same into subject in need thereof

In vitro assays are described herein which may be utilized by the skilled artisan to further or additionally screen, assess, and/or verify the activities of modulatory agents identified using screening methods as described herein. Cell based assays and in vitro methods are described herein and were utilized to perform experiments as described, for example, in the Examples.

In vivo animal models of human inflammatory diseases linked to Th17 cell-mediated pathology may also be utilized by the skilled artisan to further or additionally screen, assess, and/or verify the activity of modulatory agents identified using screening methods as described herein. Such animal models include models of human autoimmune or inflammatory diseases. An exemplary animal model system is the Th17-dependent disease model of experimental autoimmune encephalomyelitis (EAE), which mimics the CNS pathology observed in multiple sclerosis. Indeed, the EAE model was used to validate the role of Fos12 in Th17 mediate pathology in vivo. As described herein and shown in FIG. 6, Fos12^{fl/fl} CD4-Cre mice had significantly attenuated disease severity compared to wild-type controls. Analysis of spinal cord infiltrates at 21 days post immunization revealed reduced CD4⁺ T cells, but similar percentages of IL-17A, IFNγ, and GM-CSF producers, in mutant mice. Fos12-deficient cytokine-producing T-helper cells also expressed the TF Foxp3, which specifies the Treg program (FIG. 6B, C). These results are consistent with the in vitro observations (FIG. 6A) and confirm that Fos12 is a key regulator of T-helper lineage plasticity, particularly under inflammatory conditions.

Modulatory agents identified or verified in screens described herein may be administered to a patient in need of treatment via any suitable route, including by intravenous, intraperitoneal, intramuscular injection, or orally. The precise dose will depend upon a number of factors, including whether the proteins, peptides, immune activators or agents are for diagnosis or for treatment or for prevention. The dosage or dosing regime of an adult patient may be proportionally adjusted for children and infants, and also adjusted for other administration or other formats, in proportion for example to molecular weight or immune response. Administration or treatments may be repeated at appropriate intervals, at the discretion of the physician.

Modulatory agents identified or verified in screens described herein are generally administered in the form of a pharmaceutical composition, which may comprise at least one component in addition to the proteins, peptides, immune activators or agents. Pharmaceutical compositions according to the present invention, and for use in accordance with the present invention, may comprise, in addition to active ingredient, a pharmaceutically acceptable excipient, carrier, buffer, stabiliser or other materials known to those skilled in the art. Such materials should be non-toxic and should not interfere with the efficacy of the active ingredient. The precise nature of the carrier or other material will depend on the route of administration, which may be oral, or by injection, e.g. intravenous, or by deposition at a tumor site.

The mode of administration of a pharmaceutical composition comprising a modulatory agent/s identified using screening methods described herein may be by any suitable route which delivers a therapeutically effective amount amount of the agent to the subject. One such route is the parenteral route, such as by intramuscular or subcutaneous administration. Other modes of administration may also be employed, where desired, such as the mucosal route, such as by oral, rectal, buccal or intranasal administration, or via other parenteral routes, i.e., intradermally, intravenously, intraperitoneally, or intratumorally.

Pharmaceutical compositions for oral administration may be in tablet, capsule, powder or liquid form. A tablet may comprise a solid carrier such as gelatin or an adjuvant. Liquid pharmaceutical compositions generally comprise a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

For intravenous injection, or injection at the site of affliction, the active ingredient may be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilizers, buffers, antioxidants and/or other additives may be included, as required.

A composition may be administered alone or in combination with other treatments, therapeutics or agents, either simultaneously or sequentially dependent upon the condition to be treated. In addition, the present invention contemplates and includes compositions comprising the modulatory agents identified or verified in screens described herein and other agents or therapeutics such as immune modulators, antibodies, immune cell stimulators, or adjuvants. In addition, the composition may be administered with hormones, such as dexamethasone, immune modulators, such as interleukins, tumor necrosis factor (TNF) or other growth factors, colony stimulating factors, or cytokines which stimulate the immune response and reduction or elimination of virus. The composition may also be administered with, or may include combinations along with immune cell antigen antibodies or immune cell modulators.

The preparation of therapeutic compositions which contain polypeptides, analogs or active fragments as active ingredients is well understood in the art. Typically, such compositions are prepared as injectables, either as liquid solutions or suspensions. However, solid forms suitable for solution in, or suspension in, liquid prior to injection can also be prepared. The preparation can also be emulsified. The active therapeutic ingredient is often mixed with excipients which are pharmaceutically acceptable and compatible with the active ingredient. Suitable excipients are, for example, water, saline, dextrose, glycerol, ethanol, or the like and combinations thereof. In addition, if desired, the composition can contain minor amounts of auxiliary substances such as wetting or emulsifying agents, pH buffering agents which enhance the effectiveness of the active ingredient.

A modulatory agent identified or verified in screens described herein can be formulated into the therapeutic composition as a neutralized pharmaceutically acceptable salt forms. Pharmaceutically acceptable salts include the acid addition salts (formed with the free amino groups of the polypeptide or antibody molecule) and which are formed with inorganic acids such as, for example, hydrochloric or phosphoric acids, or such organic acids as acetic, oxalic, tartaric, mandelic, and the like. Salts formed from the free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like

Accordingly, also encompassed herein is a composition comprising at least one modulatory agent identified or verified in screens described herein and a pharmaceutically acceptable buffer, for use in treating a patient with a Th17 cell-mediated pathology (such as, e.g., Crohn's Disease, psoriasis, or multiple sclerosis), wherein said composition alleviates symptoms of the disease or condition in the patient when administered to the patient in a therapeutically effective amount. Such compositions may also have utility for use in prophylaxis for a patient at risk for developing a Th17 cell-mediated pathology, wherein said composition prevents or alleviates symptoms in the patient when administered to the patient in an effective amount. Also encompassed herein is the use of a therapeutically effective amount of a composition comprising at least one modulatory agent identified or verified in screens described herein and a pharmaceutically acceptable buffer in the manufacture of a medicament for treating a patient with a Th17 cell-mediated pathology, wherein the medicament alleviates or prevents symptoms of the disease or condition when administered to the patient. Also encompassed herein is at least one modulatory agent identified or verified in screens described herein and compositions thereof for use in treating an inflammatory condition or autoimmune disease in a subject.

The peptide or agent containing compositions are conventionally administered intramuscularly, intravenously, as by injection of a unit dose, or orally, for example. The term “unit dose” when used in reference to a therapeutic composition of the present invention refers to physically discrete units suitable as unitary dosage for humans, each unit containing a predetermined quantity of active material calculated to produce the desired therapeutic effect in association with the required diluent; i.e., carrier, or vehicle.

The compositions are administered in a manner compatible with the dosage formulation, and in a therapeutically effective amount. The quantity to be administered depends on the subject to be treated, capacity of the subject's immune system to utilize the active ingredient, and degree of activation and immune response desired. Precise amounts of active ingredient required to be administered depend on the judgment of the practitioner and are peculiar to each individual. Suitable regimens for initial administration and follow on administration are also variable, and may include an initial administration followed by repeated doses at appropriate intervals by a subsequent injection or other administration.

Pharmaceutical compositions for oral administration may be in tablet, capsule, powder or liquid form. A tablet may comprise a solid carrier such as gelatin or an adjuvant. Liquid pharmaceutical compositions generally comprise a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

For intravenous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilizers, buffers, antioxidants and/or other additives may be included, as required.

### Nucleic Acids

The present invention further provides an isolated nucleic acid encoding a protein, peptide, or agent of the present invention. Nucleic acid includes DNA and RNA. In a preferred aspect, the present invention provides a nucleic acid which codes for a polypeptide of the invention as defined above, including any one of SEQ ID NO: 1 or a fragment thereof as set out herein.

The present invention also provides constructs in the form of plasmids, vectors, and transcription or expression cassettes which comprise at least one polynucleotide as above. The present invention also provides a recombinant host cell which comprises one or more constructs as above. A nucleic acid encoding any specific binding member as provided herein forms an aspect of the present invention, as does a method of production of the specific binding member which method comprises expression from encoding nucleic acid therefor. Expression may conveniently be achieved by culturing recombinant host cells containing the nucleic acid under appropriate conditions. Following production by expression, a specific binding member may be isolated and/or purified using any suitable technique, then used as appropriate.

Systems for cloning and expression of a polypeptide in a variety of different host cells are well known. Suitable host cells include bacteria, mammalian cells, yeast and baculovirus systems. Mammalian cell lines available in the art for expression of a heterologous polypeptide include Chinese hamster ovary cells, HeLa cells, baby hamster kidney cells, NSO mouse melanoma cells and many others. A common, preferred bacterial host is E. coli. The expression of antibodies and antibody fragments in prokaryotic cells such as E. coli is well established in the art.

Suitable vectors can be chosen or constructed, containing appropriate regulatory sequences, including promoter sequences, terminator sequences, polyadenylation sequences, enhancer sequences, marker genes and other sequences as appropriate. Vectors may be plasmids, viral e.g. phage, or phagemid, as appropriate. For further details see, for example, Molecular Cloning: a Laboratory Manual: 2nd edition, Sambrook et al., 1989, Cold Spring Harbor Laboratory Press. Many known techniques and protocols for manipulation of nucleic acid, for example in preparation of nucleic acid constructs, mutagenesis, sequencing, introduction of DNA into cells and gene expression, and analysis of proteins, are described in detail in Short Protocols in Molecular Biology, Second Edition, Ausubel et al. eds., John Wiley & Sons, 1992. The disclosures of Sambrook et al. and Ausubel et al. are incorporated herein by reference.

Thus, a further aspect of the present invention provides a host cell containing nucleic acid as disclosed herein. A still further aspect provides a method comprising introducing such nucleic acid into a host cell. The introduction may employ any available technique. For eukaryotic cells, suitable techniques may include calcium phosphate transfection, DEAE-Dextran, electroporation, liposome-mediated transfection and transduction using retrovirus or other virus, e.g. vaccinia or, for insect cells, baculovirus. For bacterial cells, suitable techniques may include calcium chloride transformation, electroporation and transfection using bacteriophage. The introduction may be followed by causing or allowing expression from the nucleic acid, e.g. by culturing host cells under conditions for expression of the gene. The present invention also provides a method which comprises using a construct as stated above in an expression system in order to express a specific binding member or polypeptide as above.

Another feature of this invention is the expression of DNA sequences contemplated herein, particularly those encoding TRN proteins, domains, fragments, or peptides thereof, or an agent of the invention. As is well known in the art, DNA sequences may be expressed by operatively linking them to an expression control sequence in an appropriate expression vector and employing that expression vector to transform an appropriate unicellular host. A wide variety of host/expression vector combinations may be employed in expressing the DNA sequences of this invention. Useful expression vectors, for example, may consist of segments of chromosomal, non-chromosomal and synthetic DNA sequences. Suitable vectors include derivatives of SV40 and known bacterial plasmids, e.g., E. coli plasmids col El, pCR1, pBR322, pMB9 and their derivatives, plasmids such as RP4; phage DNAs, e.g., the numerous derivatives of phage λ, e.g., NM989, and other phage DNA, e.g., M13 and filamentous single stranded phage DNA; yeast plasmids such as the 2μ plasmid or derivatives thereof; vectors useful in eukaryotic cells, such as vectors useful in insect or mammalian cells; vectors derived from combinations of plasmids and phage DNAs, such as plasmids that have been modified to employ phage DNA or other expression control sequences; and the like.

Any of a wide variety of expression control sequences (sequences that control the expression of a DNA sequence operatively linked to it) may be used in these vectors to express the DNA sequences of this invention. Such useful expression control sequences include, for example, the early or late promoters of SV40, CMV, vaccinia, polyoma or adenovirus, the lac system, the trp system, the TAC system, the TRC system, the LTR system, the major operator and promoter regions of phage λ, the control regions of fd coat protein, the promoter for 3-phosphoglycerate kinase or other glycolytic enzymes, the promoters of acid phosphatase (e.g., Pho5), the promoters of the yeast-mating factors, and other sequences known to control the expression of genes of prokaryotic or eukaryotic cells or their viruses, and various combinations thereof

The invention also provides TRN chimeric or fusion proteins. As used herein, a TRN “chimeric protein” or “fusion protein” comprises a TRN polypeptide operatively linked to a non-TRN polypeptide. A “TRN polypeptide” refers to a polypeptide having an amino acid sequence corresponding to a full length TRN polypeptide or a domain or fragment thereof, whereas a “non-TRN polypeptide” refers to a polypeptide having an amino acid sequence corresponding to a protein which is not substantially homologous to the particular TRN protein. In a particular embodiment, a TRN fusion protein comprises at least one biologically active portion of a TRN protein, e.g., a transcriptional domain of a TRN protein. Within the fusion protein, the term “operatively linked” is intended to indicate that the TRN polypeptide and the non-TRN polypeptide are fused in-frame to each other. The non-TRN polypeptide can be fused to the N-terminus or C-terminus of the TRN polypeptide.

For example, in one embodiment, the fusion protein is a GST-TRN protein in which the TRN sequences are fused to the C-terminus of the GST sequences. In another embodiment, the fusion protein is a TRN-HA fusion protein in which the TRN nucleotide sequence is inserted in a vector such as pCEP4-HA vector (Herrscher, R. F. et al. (1995) Genes Dev. 9:3067-3082) such that the TRN sequences are fused in frame to an influenza hemagglutinin epitope tag. Such fusion proteins can facilitate the purification of a recombinant TRN protein.

A fusion protein comprising a TRN protein or fragment thereof can be produced by recombinant expression of a nucleotide sequence encoding a first peptide having TRN protein activity, and a nucleotide sequence encoding a second peptide corresponding to a moiety that alters the solubility, affinity, stability or valency of the first peptide, for example, an immunoglobulin constant region. In a particular embodiment, the first peptide consists of a portion of the TRN polypeptide. The second peptide can include an immunoglobulin constant region, for example, a human Cγ1 domain or Cγ4 domain (e.g., the hinge, CH2 and CH3 regions of human IgCγ1, or human IgCγ4, see e.g., Capon et al. U.S. Pat. Nos. 5,116,964; 5,580,756; 5,844,095 and the like, incorporated herein by reference in its entirety). A resulting fusion protein may have altered TRN protein solubility, binding affinity, stability and/or valency (i.e., the number of binding sites available per molecule) and may increase the efficiency of protein purification. Fusion proteins and peptides produced by recombinant techniques can be secreted and isolated from a mixture of cells and medium containing the protein or peptide. Alternatively, the protein or peptide can be retained cytoplasmically and the cells harvested, lysed and the protein isolated. A cell culture typically includes host cells, media and other byproducts. Suitable media for cell culture are well known in the art. Protein and peptides can be isolated from cell culture media, host cells, or both using techniques known in the art for purifying proteins and peptides. Techniques for transfecting host cells and purifying proteins and peptides are known in the art.

A fusion protein comprising a TRN protein or fragment thereof is produced by standard recombinant DNA techniques. For example, DNA fragments coding for the different polypeptide sequences are ligated together in-frame in accordance with conventional techniques, for example employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in Molecular Biology, eds. Ausubel et al. John Wiley & Sons: 1992). Moreover, many expression vectors are commercially available that already encode a fusion moiety (e.g., a GST polypeptide or an HA epitope tag). A TRN protein encoding nucleic acid can be cloned into such an expression vector such that the fusion moiety is linked in-frame to the TRN protein.

In another embodiment, the fusion protein is a TRN protein containing a heterologous signal sequence at its N-terminus. In certain host cells (e.g., mammalian host cells), expression and/or secretion of a TRN protein can be increased through use of a heterologous signal sequence.

A wide variety of unicellular host cells are also useful in expressing the DNA sequences of this invention. These hosts may include well known eukaryotic and prokaryotic hosts, such as strains of E. coli, Pseudomonas, Bacillus, Streptomyces, fungi such as yeasts, and animal cells, such as CHO, YB/20, NSO, SP2/0, R1.1, B-W and L-M cells, African Green Monkey kidney cells (e.g., COS 1, COS 7, BSC1, BSC40, and BMT10), insect cells (e.g., Sf9), and human cells and plant cells in tissue culture.

It will be understood that not all vectors, expression control sequences and hosts will function equally well to express the DNA sequences of this invention. Neither will all hosts function equally well with the same expression system. However, one skilled in the art will be able to select the proper vectors, expression control sequences, and hosts without undue experimentation to accomplish the desired expression without departing from the scope of this invention. In selecting an expression control sequence, a variety of factors will normally be considered. These include, for example, the relative strength of the system, its controllability, and its compatibility with the particular DNA sequence or gene to be expressed, particularly as regards potential secondary structures. Suitable unicellular hosts will be selected by consideration of, e.g., their compatibility with the chosen vector, their secretion characteristics, their ability to fold proteins correctly, and their fermentation requirements, as well as the toxicity to the host of the product encoded by the DNA sequences to be expressed, and the ease of purification of the expression products. Considering these and other factors a person skilled in the art will be able to construct a variety of vector/expression control sequence/host combinations that will express the DNA sequences of this invention on fermentation or in large scale animal culture.

The invention may be better understood by reference to the following non-limiting Examples, which are provided as exemplary of the invention. The following examples are presented in order to more fully illustrate the preferred embodiments of the invention and should in no way be construed, however, as limiting the broad scope of the invention.

### Example 1

### Materials and Methods

Mice:

Mice were bred and maintained in the animal facility of the Skirball Institute (Langone Medical Center, NYU) in specific pathogen-free conditions. C57B1/6, and Hifla^{fl/fl} (Ryan et al., 2000) mice were obtained from Jackson laboratories. Rorc(t) knock-out mice harboring a GFP reporter cDNA at the translation initiation site have been described (Eberl et al., 2004). Mutant strains were kindly provided by the following researchers: Stat3^{fl/fl} (Lee et al., 2002), D. Levy (NYU); irf4^{fl/fl}, (Klein et al., 2006) R. Dalla-Favera (Columbia University); Maf^{fl/fl} (Wende et al., 2012), C. Birchmeier (MDC, Germany); Baf^{fl/fl} (Schraml et al., 2009), K. M. Murphy (Washington University); and Fos/2^{fl/fl} (Karreth et al., 2004), E. Wagner (CNIO, Spain). Irf4^{fl/fl} mice were mated with EIIa-Cre transgenic mice to obtain fully IRF4 null animals. All animal procedures in accordance with protocols approved by the Institutional Animal Care and Usage Committee of New York University.

Cell Culture:

Naïve CD4⁺ T cells were purified by cell sorting from spleen and lymph nodes as previously described (Ivanov et al., 2006) using the Aria II (BD). Briefly, red blood cells were cleared from organ cell suspensions using ACK lysis buffer (Lonza). The resulting leukocytes were depleted of B220⁺ and CD8⁺ cells by magnetic-activated cell sorting (MACS, Miltenyi) according to the product protocol. The negative fraction was cell surface stained using antibodies specific for CD4, CD25, CD44, and CD62L, and CD4⁺CD25⁻ CD62L⁺CD44^{lo/-} naïve CD4⁺ T cells were isolated by cell sorting using the Aria II to greater than 98% purity based on post-sort analysis. Naïve CD4+ T cells were cultured in 48-, 24-, or 12-well plates coated with an anti-hamster IgG secondary antibody (MP Biomedicals), in complete IMDM media (containing 10% FCS) containing soluble anti-CD3ε (0.25 μg/mL) and anti-CD28 (1 μg/mL) for TCR stimulation. Cultures were supplemented as follows, or as indicated in figures: with anti-IL4 (2 μg/mL) and anti-IFNγ (2 μg/mL) for Th0 conditions and additionally with IL-6 (20 ng/mL; eBioscience) and TGFβ (0.3 ng/mL; PeproTech) for Th17 conditions; or 5 ng/mL TGFβ for iTreg conditions. For Th1 differentiation, IL-4 and anti-IFNγ (2 μg/mL) were added; for Th2 differentiation, IL-12 and anti-IL-4 (2 μg/mL) were added; cytokine concentrations as labeled in figures. Unless otherwise indicated antibodies were purchased from eBioscience.

Antibodies, Surface and Intracellular Staining

For analysis of cytokine production, cells were incubated for 4-5 h with phorbol 12-myristate 13-acetate (50 ng/mL; Sigma), ionomycin (500 ng/mL; Sigma), and GolgiStop (BD) at 37° C. in a tissue culture incubator. Surface cell staining was carried out with fluorescence-labeled antibodies in PBS containing 0.5% BSA and 2 mM EDTA at 4° C. for 20 min. For live cell analysis or sorting, cells were washed once in staining buffer and resuspended in 200 ng/mL of DAPI in staining buffer to exclude dead cells. For intracellular staining, cells were first stained with the fixable Aqua dead cell exclusion kit (Invitrogen), washed twice with PBS, and resuspended in Fixation-Permeabilization solution (Cytofix/Cytoperm kit; BD Biosciences or eBioscience) and intracellular staining was carried out according to the manufacturer's protocol. All fluorescence-labeled antibodies were purchased from eBioscience. An LSR II (BD Biosciences) was used for flow cytometric acquisition, followed by analysis with FlowJo software (Tree Star). All analysis plots are gated to exclude dead cells.

Chromatin Immunoprecipitation (ChIP).

TF ChIP-Seq was performed in biological duplicate as described (Johnson et al., 2007) with the following modifications. For each ChIP, 20-80 million cells were cross-linked with paraformaldehyde; chromatin was isolated and fragmented with a Vibra-Cell VCX130PB (Sonics & Materials). Following immunoprecipitation, the protein-DNA crosslinks were reversed and DNA was purified. DNA from control samples was prepared similarly but without immunoprecipitation. Histone ChIP of native chromatin was performed as previously described (Kirigin et al., 2012). Sequencing libraries were made from the resulting DNA fragments for both ChIP and controls as described (Reddy et al., 2012). The ChIP-seq libraries were sequenced with single-end 36 bp reads on an Illumina GAIIx or single-end 50 bp reads on an Illumina HiSeq 2000.

Commercial antibodies used for ChIP for each protein were as follows: IRF4 (IRF-4 M-17; Santa Cruz Biotech, sc-6059), BATF (BATF; Santa Cruz Biotech, sc-100974), STAT3 (Stat3 C-20; Santa Cruz Biotech, sc-482), p300 (p300 C-20; Santa Cruz Biotech, sc-585), Maf (Bethyl Laboratories, A300-613A), FOSL2 (Fra-2 Q-20; Santa Cruz Biotech, sc-604), HIF1α (Novus, NB100-105), ETV6 (TEL; Santa Cruz Biotech, sc-8546), JMJD6 (abcam, ab64575), NRF2 (H-300, Santa Cruz Biotech, sc-13032), H3K4me2 (Millipore, 07-030), and H3K4me3 (Millipore, 05-745R). The anti-RORγ rabbit polyclonal antibody was raised against amino acids 79-301 (Covance) and affinity purified antibody was isolated from serum using the same immunogen. The JMJD3 affinity purified antibody was kindly provided by G. Natoli (IFOM-IEO, Italy). The specificity of each transcription factor antibody was validated by immunoblot or conventional ChIP assay comparing wild-type to factor-deficient (or knock-down) Th17 subset polarized cells. In addition, ChIP-Seq was performed in knock-out cells for the core TFs to provide an additional negative control for each ChIP-seq.

FAIRE-seq

FAIRE was performed as previously described (Simon et al., 2012). FAIRE reads were mapped using Bowtie (-k 1—best) (Langmead et al., 2009) on the Galaxy platform (Goecks et al., 2010). For visualization of FAIRE signal around pCRMs, normalized alignment files were prepared using HOMER (Heinz et al., 2010), and heatmaps were made using SEQMINER (Ye et al., 2011).

Co-Immunoprecipitations

Naïve CD4 positive T cells were sorted and cultured under Th17 polarizing conditions for 48 h prior to assay. Whole cells lysates were prepared with high salt buffer (10 mM Tris, 420 mM NaCl, 0.5% NP40, 1 mM EDTA), sonicated, spun to remove insoluble particles, and diluted to a final concentration of 150 mM NaCl for co-immunoprecipitation. Endogenous IRF4 was immunoprecipitated using anti-IRF4 antibody in the presence or absence of 50 ug/ml ethidium bromide. Co-IP pulled-downs were resolved by SDS electrophoresis and anti-BATF (Santa Cruz Biotech) and STAT3 (Cell Signaling) antibodies were used for western blot detection.

Luciferase Assay

pCRM activity was assessed using luciferase reporter assays by cloning the ChIP-defined genomic region (average of approx. 750 bp) upstream of a minimal promoter driving a luciferase gene (pGL4.23[luc2/minP]; Promega). Importantly, pCRMs were selected in a non-biased manner based on ranked average binding ChIP p-values for occupying TFs; genomic coordinates are as follows:

| TF order | pCRM genomic coordinates |
|---|---|
| 1 | chr3: 78762692-78763270 |
| 1 | chr1: 43196384-43196904 |
| 1 | chr18: 75739077-75739648 |
| 1 | chr5: 138093690-138094176 |
| 1 | chr10: 94880166-94880778 |
| 2 | chr17: 55841782-55842285 |
| 2 | chr11: 109482382-109482946 |
| 2 | chr11: 44503354-44503971 |
| 2 | chr3: 103188727-103189193 |
| 3 | chr1: 155559667-155560272 |
| 3 | chr1: 184030446-184030992 |
| 3 | chr4: 59821894-59822386 |
| 3 | chr12: 33858479-33858979 |
| 4 | chr15: 9457605-9458144 |
| 4 | chr11: 44449580-44450262 |
| 4 | chr12: 101985209-101985820 |
| 4 | chr14: 52642471-52643107 |
| 5 | chr1: 20730409-20731129 |
| 5 | chr1: 20730409-20731129 |
| 5 | chr5: 53980000-53980448 |
| 5 | chr1: 146086088-146086770 |
| 5 | chr5: 53881767-53882558 |
| 5 | chr9: 107215041-107215628 |
| 5 | chr12: 74988875-74989601 |
| 5 | chr13: 16733224-16734291 |

pCRM activity was assessed using luciferase reporter assays by cloning the ChIP-defined genomic region upstream of a minimal promoter driving a luciferase gene (pGL4.23[luc2/minP]; Promega). Naïve CD4⁺ T cells were sorted and cultured under Th2, or Th17 polarizing conditions for 48 h prior to being harvested for electroporation. Briefly, 5 million cells were pre-incubated with 10 μg of pCRM-pGL4minP or empty pGL4minP construct and 2 μg of renilla luciferase plasmid in 500 μl of RPMI on ice. Cells were electroporated using a BioRad Electroporator at 300V and 750 g. After 10 min of recovery on ice, cells were placed into pre-warmed polarizing culture medium under TCR and cytokine stimulation conditions (Th2 or Th17). 24 h post electroporation, cells were collected and luciferase assays were performed using the Dual Luciferase Reagents (Promega). Firefly luciferase values were normalized to renilla luciferase values for each sample and expressed as fold change over empty pGL4-minP. pGL4-minP harboring regions from the Il17a locus: Il17a-5 (a known enhancer) (Wang et al., 2012) and Il17a-19 (a non-TF occupied conserved region 19 kb upstream of TSS) served as positive and negative controls, respectively.

### Preparation of RNA-Seq Libraries

mRNA was prepared from total RNA by poly-A selection and cDNA synthesis was carried out as described (Mortazavi et al., 2008). The resulting dsDNA was prepared for sequencing by ligation of Illumina sequencing adapters, selection of 225 bp fragments from a 2% agarose SizeSelect E-Gel (Invitrogen), and amplification with 15 cycles of PCR using Illumina paired-end primers. Alternatively, some libraries were made using the Nextera tagmentation protocol described (Gertz et al., 2012). The RNA-seq libraries were sequenced with single-end 36 bp reads on an Illumina GAIIx or single-end 50 bp reads on an Illumina HiSeq 2000. Biological duplicates were carried out for each experiment. Sequence reads were mapped to the mus musculus genome (version mm9) with Bowtie (version 0.12.7) and with the following settings: -k 1—best. The —phred33-quals or —phred64-quals parameter was set as needed depending on the format of the input fastq file. Anywhere between 8.5M and 78.7M reads aligned per library. Read counts for annotated genomic features were computed using the htseq-count script from the HTSeq (version 0.5.3p3) software suite with parameters: —stranded=no —mode=union.

siRNA Knock-Downs

For knockdown of network genes in T cell polarization cultures, naïve C57/B16 CD4 T cells were sort purified and cultured for 16-18 h in RPMI/Th0 conditions. 2 million stimulated cells were transfected with 300 pmol of control siRNAs for Ccr6, Rorc, and a non-Targeting pool (pool #2; SMARTpool siRNA; Dharmacon), in addition to SMARTpool siRNAs for network target genes (Dharmacon). Transfections were performed using the Amaxa Mouse T cell Nucleofector Kit with the X-001 program (Amaxa) according the manufacture's protocol. After a 4 h recovery at 37° C., cells were stimulated in Th17 conditions in RPMI media. RNA was prepared from cells collected at 24 h post polarization to assess knockdown efficiency and for RNA-Seq. Flow cytometric analysis for IL-17A and Foxp3 24 h post polarization; viability was assessed by Aqua exclusion (Invitrogen) and cell counts by Accucount particles.

Collection of GWAS and SNP Data for Network Validation:

Disease-associated SNPs compiled from the National Human Genome Research GWAS Catalog (available at www.genome.gov/gwastudies; accessed Feb. 29, 2012). For each condition, gene lists were produced by selecting catalog-annotated human genes within 100 kb of associated SNPs. In cases where a SNP falls between two loci, the closest gene was chosen for association. Gene lists were used with no regard to human-mouse synteny.

Retroviral Gene Transfer

Retroviral constructs were generated by subcloning of the cDNA of interest into MSCV-Thy1.1 5′ of the internal ribosomal entry site, permitting the bicistronic expression of candidate TFs and cell surface Thy1.1. Retrovirus was generated by transfection of retroviral constructs into the PlatE producer cell line (Morita et al., 2000); viral supernatants were used at 48 h post transfection. FACS sorted naïve CD4 T cells were stimulated under Th0 conditions for 20-24 h prior to retroviral transduction. For gene transfer, cells were spin transduced for 2 hr at 2500 rpm with viral supernatants in the presence of 6.7 ug/mL of polybrene (hexadimethrine bromide, Sigma), and media was replaced with T cell polarization media for differentiation to Th17 and control Th1. Cells were harvested after 48 h (Th17) and 5 days (Th1) for flow cyotmetric analysis of cytokine production.

EAE Induction

For induction of EAE, mice were immunized subcutaneously on day 0 with 200 μg/mouse MOG 35-55 peptide (UCLA peptide synthesis facility), emulsified in CFA (CFA supplemented with 2 mg/ml Mycobacterium tuberculosis), and injected intravenously on days 0 and 2 with 200 ng/mouse of pertussis toxin (Sigma Aldrich). The following scoring system used was 0—no disease, 1—limp tail, 2—weak/partially paralyzed hind legs, 3—completely paralyzed hind legs, 4—complete hind and partial front leg paralysis, 5—complete paralysis/death. Mice with disease levels 4 and 5 were considered moribund and were euthanized.

Isolation of Mononuclear Cells from Spinal Cords

Before spinal cord (SC) dissection, mice were perfused with 30 ml of cold Ca²⁺/Mg²⁺-free PBS. The spinal columns were dissected, cut open, and intact SCs separated carefully from the vertebrae. The SCs were cut into several small pieces and placed in 2 ml digestion solution containing 10 mg/ml Collagenase D (Roche) in PBS with 5% FCS. Digestion was performed for 30 min at 37° C. Digestion was terminated by the addition of EDTA to a final concentration of 12.5 mM and an additional 5 minute incubation. The resulting digested tissue was passed through a 70 um cell screen. The cells were washed once in PBS, placed in 10 ml of 38% Percoll solution, and pelleted for 30 min at 2000 rpm with no brake. Cells pellets were washed once in PBS, re-suspended in FACS buffer or T cell medium and stimulated for assessment of cytokine production and Foxp3 expression as described above.

### Computational Methods:

Primary data processing of ChIP-seq and RNA-seq experiments Sequence reads were mapped to the mus musculus genome (version mm9) with Bowtie (version 0.12.7) (Langmead et al., 2009) and with the following settings: -k 1—best. The —phred33-quals or —phred64-quals parameter was set as needed depending on the format of the input fastq file. Anywhere between 8.5M and 78.7M reads aligned per library. ChIP-seq datasets were further processed to call peaks with the MACS software (version 1.4.0 20110619) using the settings: -p le-10-m 15, 30-s 36-g mm —bw=200 (Zhang et al., 2008). All were processed against an appropriate control. RNA-seq datasets were also processed through Tophat (version 1.2.0) with settings: -a 10-g 20 —no-novel-juncs -G refseqGeneArtnot.gtf (Trapnell et al., 2009). Tophat results were then pipelined to Cufflinks (version 0.9.3) with the settings: -M 20101217_rRNA_tRNA_mask.gtf -G refseqGeneAnnot.gtf (Trapnell et al., 2010). Absolute read counts for annotated genomic features were computed using the htseq-count script from the HTSeq (version 0.5.3p3) software suite with parameters: —stranded=no —mode=union.

### Network Inference Via Integration of ChIP-Seq, RNA-Seq and Microarray Data.

Overview of Integrative Network Inference:

Here we describe how we scored TF→target gel regulatory interactions based on the four main complementary data types that include the majority of the data collected in this study. We integrate: 1) ChIP-seq for TFs, 2) RNA-seq following knock-out of TFs, 3) RNA-seq of Th17 differentiation (time series) and steady state data for other CD4+subsets, and, 4) Immgen data, a publically available microarray compendium spanning the hematopoietic differentiation tree (Heng and Painter, 2008). We combine these data types into a multi-support directed regulatory network that accurately predicts the regulatory events responsible for specifying the Th17 lineage. Recent work has clearly demonstrated the utility of combining data types as diverse as TF binding, motif conservation, and chromatin modifications for prediction of regulatory interactions (Marbach et al., 2012a; Ouyang et al., 2009; Park et al., 2005; Zhou et al., 2010). Note that we used an older release of the Immgen dataset (dated to March 2011) as the Immgen rules require users not to publish results based on data within six months of its release.

Structure:

In the next four sections we describe how we calculated TF→target gel confidence scores for each individual data source using a method of our own construction. For each data source we store the confidence scores in an M×N matrix, S(Dᵢ), where M is the number of genes, N is the number of TFs, and

Dᵢε[KO, ChIP, RNAseq, Immgen] is the data-type in question. Then we map confidence scores (p-values for ChIP and knock-out, or pseudo z-scores for RNA-seq and Immgen) for each matrix, S(Dᵢ), to rank-based quantile scores that we store in an M×N matrix, Q(Dᵢ). The previous step is required for data integration. Thereafter we show how we integrated scores over multiple data sources by summing (element by element) Σ_{D}_{ᵢ}_{εD′}Q(Dᵢ), D′⊂[KO, ChIP, RNAseq, Immgen], for any data combination we tested in this work.

### ChIP-seq, Defining TF→Target Gene Association Scores:

Scoring of a given TF's association with a target gene in a given ChIP-seq experiment is usually defined (at least in part) based on TF-binding site proximity to a target gene's transcription start site (TSS), and is often given a binary value indicating if a regulatory interaction exists or not (Boyer et al., 2005; Chen et al., 2008; Marson et al., 2008). However, these commonly used formulations discount the majority of TF binding data that is not proximal to TSS and may be important for regulation, and do not provide a continuous score which is needed for ranking possible target genes by confidence. Recent works have started to address these limitations by considering wider regions around the TSS of genes, and assign a continuous confidence score for putative TF-target gene interactions (Ouyang et al., 2009). Here, we use a TF-gene association score that includes regulatory regions that are far from the promoter, as we know that active regulatory elements are often in introns or distal regulatory regions. We thus examined the full gene (TSS to end of last exon) plus 10 kb on each side and used a scoring scheme that integrated all peaks found for a given TF in that region, normalizing for the number of peaks of similar strength expected by chance. It is important to note that we primarily use this p-value score as part of a larger integrative framework (integrated with KO and time series expression data) to form our final network; we can thus initially trade sensitivity for accuracy and recover accuracy via our subsequent integration with other data types.

Let g be a possible target gene of TF x and L_{g} be the genomic region surrounding gene g (as defined above, gene+/−10 kb). Let |L_{g}| be the genomic span of L_{g} in bps. Also, let Sₓ be the set of peaks identified by MACS for TF across the genome, and Sₓ^{g} be a subset of that denotes those peaks that locate in L_{g}. Assuming a naïve null hypothesis that peaks are distributed randomly across the genome, the probability of observing |Sₓ^{g}| peaks in L_{g} follows the Poisson distribution with an expected number of occurrences λ=|L_{g}|×ρ(x), where ρ(x) is the expected number of peaks per by for TF x. A simple way to estimate ρ(x) is to divide the total number of peaks by the genome size,

$$ρ ( x ) = \frac{\vert {S}_{x} \vert}{G} .$$

Then the probability of observing |Sₓ^{g}| peaks in L_{g}, is, Poisson(n≧|Sₓ^{g}|,λ=|L_{g}|×ρ(x)). However, this simple formulation does not differentiate between a gene region that has n strong peaks and the same gene region with □ weak peaks. We thus calculated ρ(x) in a manner that would incorporate the binding significance of peaks found in L_{g}, as follows:

$$ρ ( x ) = \frac{\vert α ∈ \text{?} - {log}_{10} pval ( a ) \geq mean ( - {log}_{10} pval ( \text{?} ) ) \vert}{G}$$

$$\text{?} \text{indicates text missing or illegible when filed}$$

where G is the mappable size of the genome, and the numerator specifies the total number of peaks (genome-wide) with significance equal or greater to the average significance of peaks found in the region of L_{g}. We then defined the TF-gene association score s to be −log₁₀ of the p-value of observing Sₓ^{g} in L, given ρ(x), which can be calculated as:

s(x→g|ChIpseq x)=−log₁₀(Poisson(n≧|Sₓ^{g}|,λ=|L_{g}|×ρ(x))).

Although we chose the above ChIP scoring scheme (which considers the entire gene body +/−10 kb) to achieve greater sensitivity, our subsequent computational and experimental validation of our network models revealed that this gene-wide ChIP-seq scoring scheme was overall more successful than a more traditional TSS proximal scoring scheme (considering a region of +/−5 kb around TSS) at identifying known Th17 target genes as top scoring hits, as measured by both the area-under-curve of precision recall (accuracy) and Receiver operator curves (sensitivity), respectively (data not shown).
RNA-Seq (Wild Type Vs. Knock Out), Defining TF→Target Gene Association Scores from TF KO RNA-seq Data:

To determine TF→target gene associations scores based on knock-out data, we performed RNA-seq knock-out experiments for key TFs (same TFs as in the ChIP-seq experiments) under Th17 stimulating conditions. Let x denote a knocked-out TF and g denote a putative target gene. For each gene g under x wild-type vs. knock-out conditions we computed a fold change, and a corresponding p-value using DEseq (Anders and Huber, 2010), a program to calculate the significance of differential expression from RNA-seq. We then used −log₁₀ of the p-value reported by DEseq as a confidence score for the association between TF x and gene g:

$$s ( x \to g \vert knockout x ) = - {log}_{10} ( \text{?} ( \frac{reads ( ( \text{?} ) )}{reads ( ( \text{?} ) )} ) \times sign ( {log}_{2} ( \frac{reads ( ( \text{?} ) )}{reads ( ( \text{?} ) )} ) ) )$$

$$\text{?} \text{indicates text missing or illegible when filed}$$

where reads(g(x^{wt})) and reads(g(x^{kc})) denote the number of reads sequenced corresponding to the mRNA of gene g in wild-type and knockout cells, respectively (adjusted for differences in library size). Note that we multiplied each regulatory interaction confidence score by the sign of the fold change to indicate activating from repressing interactions (positive and negative scores, respectively). Also note that individual pairs of knock-out vs. wild-type experiments of at least two biological repeats were run separately through DEseq to determine p-values from each pair, which were then combined using Fisher's method for combining p-values (Fisher, 1925). We used this meta-analysis procedure since we found that inherent systematic biases between biological replicates (such that the knock-out of one experiment is more correlated with its corresponding wild-type control than with the knock-out of the other replicate) can significantly degrade DEseq performance (data not shown).
Using the Inferelator to Derive Networks from Our RNA-Seq Data Compendium (Time-Series, Knock-Outs, and Other CD4+Lineages):

We collected various RNA-seq experiments including time series for Th17 (or Th0 as a control) specification in vitro, knockouts as described above, and additional RNA-seq for alternative CD4+lineages. This resulted in a Th17 and T-cell focused compendium of 155 RNA-seq experiments. We used our RNA-seq data and the 2011 version of the ImmGen dataset as input to the Inferelator to learn additional regulatory relationships (thus expanding the coverage of our network). The Inferelator can also provide further support for regulatory relationships that were learned from the knock out and ChIP data (complementary estimates of the strength, timing, and directionality of these interactions). We have previously shown that the Inferelator is an effective (top performing when compared to many alternative methods) general method for leveraging diverse data types, such as time-series and knockouts, to learn global transcriptional regulatory networks (Bonneau et al., 2007; Bonneau et al., 2006; Gilchrist et al., 2006; Madar et al., 2009; Madar et al., 2010; Marbach et al., 2012b). For a detailed description of the current method we refer the reader to (Madar et al., 2010).

The current version of the Inferelator is composed of two core methods that we have shown to be mutually reinforcing: time-lagged Context Likelihood of Relatedness (tlCLR) (Madar et al., 2010), an extension of the CLR method (Faith et al., 2007) that explicitly uses time series data alongside steady-state data for computing time-lagged mutual information, and the Inferelator 1 (Bonneau et al., 2006). which learns regulatory dynamics as well as network topology by explicitly using time-series data to parameterize a linear ordinary differential expression model using the elastic net (Zou and Hastie, 2005), an l1- and l2-norm constrained model selection method. The Inferelator takes as input a genome-wide data set of transcriptome data (typically microarrays or RNA-seq), which can contain time-series data as well as steady-state perturbation data (e.g. knockouts), and outputs a ranked list of regulatory interactions based on confidence scores. We denote the Inferelator-generated scores for TF x regulating gene g as:

s(x→g)|RNAseq)=Inferelator(x→g|RNAseq)×sign(cor(x,g)).

Note that we multiplied each regulatory interaction confidence score by the sign of the correlation coefficient between the TF and the putative target gene to differentiate putative activating from repressing interactions (positive and negative scores, respectively).
Using the Inferelator to Derive Th17 Relevant Networks from the Immgen Public Data (Multiple Immune Lineages):

The version of Immgen data we use dates to March 2011 and has expression data for over 167 distinct immune cells or conditions (Heng and Painter, 2008), not-including the Th17 cell population that we examined herein. As with the RNA-seq transcriptome data, we used the Inferelator to score regulatory interactions:

s(x→g|Immgen)=Inferelator(x→g|Immgen)×sign(cor(x,g)).

As specified above we multiplied confidence scores from the Inferelator by the correlation sign to indicate activating from repressing interactions (positive and negative scores, respectively).
Combining TF→Target Gene Scores from Multiple Data Sources:

Regulatory network inference based on any single data source alone has strong limitations that are the result of 1) the many layers of regulation comprising biological regulatory networks, 2) systematic errors associated with individual data sources, and 3) methodological constraints. In our case, ChIP-seq for a single TF will inform us of direct regulatory interactions, but these interactions may or may not be functional. Knock out data, on the other hand, will identify regulatory interactions that are functional but may or may not be direct. Correlative and time series analyses based on large compendia of transcriptome data suffer high false positive rates, due primarily to identifiability problems. The latter, although having more false positives, can still provide a boost to regulatory interactions found by ChIP-seq and KO, and more importantly provide information about regulatory information for which ChIP or KO data is not available. In our integrative regulatory network inference scheme regulatory interactions with support from multiple data sources are typically higher in accuracy than even the most confident predicted regulatory edges derived from single data-types; this is the basis for the integrative score we describe below.

When combining regulatory network scores derived from disparate data types one faces several challenges. Two strategies for combining different metrics (where each metric is a separate score of a TF->target pair) are: 1) parametric approaches such as converting each metric to a similar numerical space or metric, such as p-values or Z-scores and then performing the appropriate meta-analysis to combine metrics, and 2) converting each metric to ranks and then averaging ranks across data/support types. Recently, ranked based methods proved effective in learning regulatory networks from complementary data-sources (Madar et al., 2010; Marbach et al., 2012a; Marbach et al., 2012b; Prill et al., 2010). Rank-based methods for combining disparate measures are robust to cases where p-value (or other significance values) range over many orders of magnitude and differ in range dramatically between data sources. Here we used a relative rank (i.e. quantile) method for combining network metrics derived from four distinct data sources into a final network. Let Dᵢε[KO, ChIP, RNAseq, Immgen] be one of the data sources we integrate over, S(Dᵢ) be an M×N matrix with rows representing genes and columns TFs, and let each entry s_{gx}(Dᵢ) hold the confidence score for TF x regulating gene g based on data type Dᵢ, i.e.:

s_{gx}(Dᵢ)=S(x→g(Dᵢ).

Note that for Dᵢ=[KO, ChIP] most TFs were not measured and will thus have no regulatory information, i.e. columns in 5 that correspond to these ‘missing’ TFs will only have zero values. We then convert all non-zero confidence scores into quantile scores that range from zero (lowest confidence) to 1 (highest confidence), in a procedure that we describe below.

Let Q(Dᵢ) be an M×N matrix, with each entry, q_{g,x}(Dᵢ) equal to 1 minus the rank, in descending order, of the absolute confidence score |s_{g,x}(Dᵢ)|. by the total number of non-zero scores in S(Dᵢ):

$${q}_{g , x} ( {D}_{i} ) = ( 1 - \frac{rank ( \vert {s}_{g , x} ( {D}_{i} ) \vert )}{\vert a ∈ S ( {D}_{i} ) \text{?} a \text{?} 0 \vert} ) \times sign ( {S}_{g , x} ( {D}_{i} ) )$$

$$\text{?} \text{indicates text missing or illegible when filed}$$

Note that under this formulation q_{g,x}(Dᵢ)ε[−1,1], where negative scores indicate repression, positive scores activation, the absolute values indicate the confidence level. All zero confidence regulatory interactions from before remain zero after mapping to quantiles.

We can now proceed to combine results over combinations of data types. Let D⁺⊂[KO,ChIP,RNAseq,Immgen] indicate the subset of data sources. We defined the data-combined M×N score matrix C(D⁺), with each entry representing the combined scores over D⁺, as:

$$\text{?} ( {D}^{*} ) = \text{?} {q}_{gx} ( {D}_{i} )$$

$$\text{?} \text{indicates text missing or illegible when filed}$$

In this manner we calculated the ranked regulatory interaction lists for each TF over every possible data combination (used in FIG. 4B).

Although this rank-based approach is simple, one complication does exist: q_{g,x}(Dᵢ=ChIP) is always greater or equal to zero but can equally indicate activation or repression (as ChIP support for a regulatory interaction is in line with both a repression and activation). Thus, when calculating c_{g,x}(D⁺) for a data combination that included ChIP, we determined the sign of the ChIP score to be in line with the sign of c_{g,x}(D⁺) with all other data types except ChIP (e.g. for c_{g,x}(D⁺=[ChIP,KO,Rnaseq,Immgen], we first determined the sign of c_{g,x}(D⁺=[KO,Rnaseq,Immgen]), and then added the ChIP score with the same sign. Note that under this integrative formulation a TF→target gene interaction that receives contradicting repressive or activation inputs from KO, RNAseq, or Immgen data, also receives a lower confidence score (i.e. the null hypothesis used is that a coherent regulation does not exist, rather than, a regulation does not exist). This consideration of regulation sign significantly boosted performance for combinations that involved the more general transcriptome data of RNAseq and Immgen (data not shown).

### Combining TF→Target Gene Scores Over Multiple TFs:

We compute a simple score that identifies genes regulated by many of the core Th17 TFs (BATF, IRF4, STAT3,c-MAF, and RORC), as these genes are more likely to be Th17 relevant. We used this simple score to prioritized genes for further study (FIG. 3B). Given our integrated TF x→gene g score c_{g,x}(D⁺), and a combination of TFs, X=(BATF, IRF4, STAT3, c-MAF, RORC), we calculated multiple TF scores as:

$$\text{?} ( {D}^{*} ) = {\sum }_{x ∈ X}^{ } {c}_{g , x} ( {D}^{*} )$$

$$\text{?} \text{indicates text missing or illegible when filed}$$

These TF-sum scores correspond to the bars shown in FIGS. 4B (for any data combination, D⁺) and 4D (for D⁺=[ChIP, KO, RNAseq, Immgen]). The majority of the Th17 relevant genes identified in our creation of the Th17 target benchmark consisted almost entirely of genes that are up regulated in Th17 cells. Therefore we use only positive network scores (activating) when calculating precision-recall with this benchmark. When repression scores are included, absolute performance is slightly decreased but the relative ranking of methods combinations is unaffected by inclusion of repressive network edges (showing that combining TFs and all four data types helps recover Th17 genes).
Comparison of a Rank-Based Meta-Analysis with Fisher's Method for Combining p-Values:

The rank based approach described above is a non-parametric statistical method. We chose it as the distribution and type of scores derived from the four data sources and methods combinations vary by several orders of magnitude in scale (FIG. S4C), and because the null hypotheses for each data type are different (e.g. a TF-gene binding does not exist for ChIP, and a TF-gene expression dependency does not exist for KO RNA-seq), hampering the use of methods that assume p-values are generated by a similar distribution resulting from comparison to the same null hypothesis. To assess if our rank-based strategy was indeed more suitable than a parametric alternative we compared its performance to Fisher's method for combining p-values (Fisher, 1925). Pseudo z-scores from inferelator were converted to p-values assuming a normal distribution to allow the Fisher's method to be applied over all data sets. Let x denote a TF and g denote a putative target gene. Let Dᵢγ[KO,ChIP,RNAseq,Immgen] be one of the data sources we integrate over, P(Dᵢ) be an M×N matrix with rows representing genes and columns TFs, and let each entry p_{g,x}(Dᵢ) hold the p-value for TF x regulating gene g based on data type Dᵢ, i.e.:

p_{g,x}(Dᵢ=pvalue(x→g|Dᵢ).

We can now proceed to combine p-values over data sources using Fisher's method as follows. Let D⁺⊂[KO,ChIP,RNSseq,Immgen] indicate a subset of data sources to combine over. We defined the data-combined M×N test statistic matrix (D⁺), with each entry representing the X² test statistic as: T_{g,x}(D⁺)=−2Σ_{D}_{ᵢ}_{εD}_{⁺}ln(p_{g,x}(Dᵢ)). We then used these test statistic scores to calculate the data integrated p-values assuming a X² distribution with k=2|D⁺| degrees of freedom, T(D⁺)˜X²(k).

We similarly combined TF→target gene p-values over TFs for a given data subset. Let X=(BATF, IRF4, STAT3, c-MAF, RORC), then the test statistic matrix is T(D⁺)=−2Σ_{xεX}_{²}ln(p_{g,x}(D⁺)), and the combined p-values can be calculated assuming a X² distribution with k=2|X| degrees of freedom, T(D⁺)˜X²(k). Results of comparing Fisher's method to the ranked based method show that Fisher's approach can be better for combining p-values within a single data source (blue bars in FIG. S4C correspond to combinations of single data-types where the performance of Fishers method is better or comparable to our rank based method). Our rank based method significantly out-performed Fisher's method when combining scores from different data sources where distributions of p-values vary (e.g. compare performance for the top performing full combination of all data , KCRI in FIG. S4C). Assigning peaks from multiple ChIP-seq experiments into putative C is Regulatory Modules (pCRMs)

We clustered peaks of multiple TFs that co-localized over small genomic regions into putative C is Regulatory Modules (pCRMs) (Chen et al., 2008). TFs peaks were joined into a single pCRM if the distance between their peak summits was less than 100 bp. Additional TF peaks were added to a growing pCRM if their summit lay within 100 bp of any peak within that pCRM. Simplified pseudo-code for this method for grouping ChIP-seq peaks into pCRMs is presented below.

n = number of TFs to be clustered into pCRMs (number of ChIP-seq
experiments)
d = user defined parameter, max distance of a summit to closest neighbor
summit in pCRM (set to 100bp in this work).
S = a list. Each element Sᵢ (i=1:N) is a vector of summits belonging to TFᵢ
M = the output list. Each element will correspond to a single pCRM
As input we have a list S of n vectors, one vector of summits per TF.
1. Combine and sort ALL summits from S into one ordered (5′ to 3′)
vector S^{ord}.
Note that S^{ord} contains summits of multiple TFs ordered by their bp
positions. We also store the name of the TF for each peak and the p-value
of the peak in identically ordered vectors.
2. Coalesce all peaks into pCRMs
For i in 1:( (length(S^{ord})−1) ) {

# if next summit is less than d bp away
If( (S^{ord} [i+1]− S^{ord} [i]) < d ) {

Add peak i+1 to current pCRM list M□

} else {

Initialize a new pCRM for peak i+1 in M

}

}

De novo Motif Detection:

TF binding DNA motifs were identified by the online version of MEME-ChIP de novo motif analysis under default parameters (Machanick and Bailey, 2011). For each TF we chose the best 500 peaks (highest −log 10 p-value), focusing each motif search on the DNA sequence that spanning the 100 bp centered at peak summit. For IRF4 and BATF motif analysis, peaks belonging to 4 sub-types of pCRMs were considered: BATF alone; IRF4 alone; BATF and IRF4 alone; and BATF, IRF4, plus additional ChIPed TFs (one or more of: STAT3, MAF, or RORC).

### Differential ChIP: Comparing ChIP-Seq for TF-x in TF-y Deficient Mice

In order to test the extent of influence TF y has on the genomic binding distribution and strength of TF x, and to assess if a p air of TFs (x, y) acts cooperatively, we compared ChIP-seq for TF x in wild-type to ChIP-seq of x in y deficient mice (knockout). Let be the set of pCRMs determined based on ChIP-seq experiments for Batf, Irf4, cMaf, Stat3, and Rorc, and define the genomic start and end by positions of each pCRM mεM as the extremum 5′ and 3′ bp positions of the individual peaks found in m. To control for indirect effects y may have on the binding profile of x (i.e. y deficient mice may have an altered expression level for x) we subdivided M into three subsets: Mₓ—the set of singleton pCRMs containing peaks only for x (here we do not expect y to have a direct influence on x), M_{xy}—the set of pCRMs containing peaks for both x and y but no other TF (here we aim to test if y has a direct effect on x), and M_{x,y}—the set of pCRMs containing peaks for both x and y and at least one additional ChIPed TF (here we aim to test how much of the effect of y on x is dependent on other factors). We can now calculate the number of reads per million (RPM) found in m for each ChIP-seq experiment: x in y wild-type background, rpm(x|y^{wt},m), and x in y deficient mice, rpm(x|y^{ko},m). For each pCRM we then determine the fold change in binding as:

$${FC}_{m} ( x \vert y ) = {log}_{2} ( \frac{rpm ( x \vert \text{?} )}{rpm ( x \vert \text{?} )} ) . \\ \text{?} \text{indicates text missing or illegible when filed}$$

We computed significance scores based on a Poisson distribution with a dynamic background model, similar to the background model scheme employed by MACS (Zhang et al., 2008). This score was used in the volcano plots shown in FIG. 2. This score accounts for the fact that some areas of the genome are generally more accessible and thus may collect more mapped reads irrespective of the ChIPed TF. Thus, we calculated lambda (the parameter in the Poisson distribution controlling the expected distribution of reads counts for a given region) based on the genome-wide number of reads or the local read count. We considered two cases depending on the sign of FCₘ(x|y). If FCₘ(x|y)≧0, i.e. in the pCRM m there was a stronger binding signal for TF x under TF y wild-type conditions, then we define:

λₘ^{dynamic}=max(λₘ^{local}(x|y^{ko}),λ_{BG}^{global}(x|y^{wt})),

where λₘ^{local}(x|y^{k0}) is the number of RPMs found in the DNA region of m for x in y knock-out background , and λ_{BG}^{global}(x|y^{wt}) is the genome-wide expected RPM given in y wild-type background.

We then define the significance of FCₘ(x|y) to be:

pₘ(x|y)=Poisson(n≧rpm(x|y^{wt},m);λₘ^{dynamic})

Conversely, if FCₘ(x|y)<0, then λₘ^{dynamic}−max(λₘ^{local}(x|y^{wt}),λ_{BG}^{global}(x|y^{ko})), and p(x|y)=Poisson(n≧rpm(x|y^{ko},m);λₘ^{dynamic}).

### Identification of Additional Th17 Core TFs:

We developed a simple procedure to use our Inferelator networks to identify additional regulators that act similarly to core TFs (BATF, IRF4, STAT3, c-MAF, and RORC). Recently, a similarly motivated method to identify master regulator TFs has been shown to be successful (Carro et al., 2010; Lefebvre et al., 2010). To this end we defined a ranked reference list of Th17 relevant genes (targets of known Th17 TFs in our networks), and queried additional TFs target repertoire for significant overlap with this list. We generated this starting Th17-relevant reference gene list from the KO and ChIP-seq network surrounding BATF, IRF4, STAT3, c-MAF, and RORC. We then determined for each query TF (within the set of several hundred TFs in the Inferelator network model), a ranked target gene list from the Inferelator generated network scores, s(x→g|Immgen). We have previously shown that TF→target gene predictions made by the Inferelator are highly accurate for top ranking predictions, and thus construct this score so that it emphasizes top ranked regulatory interaction for these core TFs. We restrict this analysis (for each TF) the top 100 to 300 TF->target pairs ranked by Inferelator score. Then, for each TF we can calculate the enrichment of these n top ranked target genes in the reference list of Th17 relevant targets. We used three metrics to determine the recovery performance significance: 1) area under curve of Precision Recall curves, 2) area under curve of Receiver Operator curve, and 3) Gene Set Enrichment analysis (Subramanian et al., 2005). All three methods return a value between 0 to 1 that determines the level of agreement between the ranked reference list and the TF top n Inferelator targets as move from top ranked predictions to the n′ th prediction (0 no enrichment), 1 (full agreement; all n genes recovered first by the ranked reference list). To determine p-values for each metric we run 20,000 simulations with a random set of n genes. The geometric mean of the three distinct p-values was used as a final score to rank TFs for further study. We chose n=200 as this value recovered the five positive control core TFs: BATF, IRF4, STAT3, c-MAF, and RORC, as top enriched TFs. This score was used to guide our iterative experimental design and was used to identify Hif1a and Fos12, as well as several of the additional TFs, as high priority candidates for second and third rounds of additional ChIP-seq, KD and KO experiments.

### Results

TF Co-Occupancy Enriches for Functional Cis Regulatory Modules

We studied early Th17 cell specification events in a largely synchronized population of naïve CD4⁺ T cell precursors induced to produce IL-17 following stimulation through the TCR in the presence of IL-6 and low levels of TGF-β (FIG. S1A). In this model, cells receiving TCR stimulation without exogenous cytokines serve as a non-polarized (Th0) control.

To assemble a high resolution map of TF-DNA interactions in Th17 differentiation, ChIP-seq experiments were performed with antibodies directed against STAT3, IRF4, BATF, c-Maf, RORγ, and p300 with cells cultured in Th0 and Th17 conditions for 48 h, a time at which Th17-specifying TFs are simultaneously expressed or active (FIG. S1B). Many high confidence bound regions were observed for each TF in Th17 conditions and for BATF and IRF4 in Th0 cells with the cognate consensus binding motif recovered for each (FIG. S1C). TFs binding at key lineage-associated loci (Il17a, Il17f, Il12rb1, IL1r1, Rorc) revealed a high degree of co-localization in Th17-polarized cells (FIG. 1A, S1D), indicating that these TFs occupy common cis regulatory regions and highlighting their roles in integrating cytokine and TCR-derived signals. In contrast, insulator-binding factor CTCF displayed a distinct occupancy pattern.

To examine genome-wide TF binding patterns in Th0 and Th17 cells, we merged TF-binding peaks with summits that clustered in close proximity to each other (within 100 bp) to define putative C is Regulatory Modules (pCRM; FIG. 1B). In this manner, 162,113 significant TF peaks clustered into 83,138 non-redundant pCRMs with distributions provided in FIG. 1C. The clustered heat map in FIG. 1B displays TF binding significance for TSS-proximal (+/−5 kb) pCRMs and the associated fold change in gene expression in Th17 versus Th0 cells for the nearest gene (right panel).

The pCRM clustering revealed that the most prominent signature was co-occupancy by all five TFs in Th17 cells and by IRF4 and BATF in Th0 cells (clusters 1 and 2, FIG. 1B). This was not a result of TF binding enrichment expected near the TSSs, as approximately 70% of the 5-TF pCRMs localized to distal sites (>5 kb from TSS; FIG. S1E). Notably, BATF and IRF4 showed a striking binding overlap regardless of occupancy by other Th17 TFs (clusters 3-6, FIG. 1B). TF average binding significance increased with pCRM order and was the highest at 5-TF pCRMs (FIG. 1C), possibly reflecting enhanced accessibility and/or cooperativity between factors at these regions. Moreover, strong RORγt binding was almost exclusively restricted to five-factor pCRMs (clusters 1 and 2), suggesting that these elements represent important regulatory domains for the integration of specification signals. Accordingly, these pCMRs were proximal to loci showing differential expression in Th17 versus Th0 cells (FIG. 1B, far right column). Nearly all (>99%) 5TF-pCRMs co-localized with the histone acetyltransferase p300 (FIG. 1B), a factor for which occupancy can be predictive of tissue-specific regulatory activity (Visel et al., 2009). Notably, p300 binding in Th17 cells, while induced relative to Th0, was not restricted to up-regulated loci, which may reflect the contribution of inhibitory gene regulation by distal pCRMs.

To relate pCRM occupancy to cis regulatory function, genomic regions corresponding to pCRMs occupied by different numbers of TFs were cloned upstream of a minimal promoter driving a luciferase reporter and assayed for activity (FIG. 1D). Among pCRMs tested, the most active regions coincided with high order occupancy that were also significantly more active in Th17 versus control Th2 cells. Thus, cis regions that integrate Th17 signals (4 and 5 TF pCRMs) display lineage selectivity and their combinatorial occupancy increases the likelihood of activity. Together, these findings support the view that core Th17 TFs function synergistically and that shared regulatory targets of STAT3, IRF4, BATF, c-Maf and RORγt are likely enriched with key targets for Th17 cell specification.

### Cooperative Binding of BATF/IRF4 Complexes Pre-Patterns Chromatin for Specification

The strong association between IRF4 and BATF occupancy in both Th17 and Th0 cells suggested a regulatory interaction between these factors. Indeed, in five-TF pCRMs, the summits for IRF4 and BATF binding were spatially more proximal than any other pair of Th17 TFs (FIG. 2A), and BATF was uniquely co-immunoprecipitated with IRF4 in a DNA-dependent manner in Th17-polarized cells (FIG. 2B), indicating that they form a complex on DNA. Consistent with this, pCRMs co-occupied by BATF and IRF4 lacked the interferon stimulated response element (ISRE) consensus, but were enriched with two dominant AP1-ISRE composite elements with AP-1 motifs adjacent to ISRE half sites (FIG. 2C). As a similar motif structure underlies cooperative binding of IRF4 and PU.1 (Eisenbeis et al., 1995), we tested functional cooperativity of BATF and IRF4 binding in a cellular context. We thus performed ChIP-seq for each TF in Th0 and Th17-polarized cells genetically deficient for the other TF. IRF4 and BATF occupancy was markedly reduced in Batf and Irf4 mutant cells, respectively. The effect was most significant at pCRMs occupied by IRF4 and BATF in combination or with additional TFs when compared to regions harboring single IRF4 or BATF peaks, where cooperativity is not expected (FIG. 2D). The dependence between IRF4 and BATF was stronger at distal pCRMs, suggesting that additional factors binding near promoters compensate for the loss of either TF. Similar results were obtained in Th0 cells in the absence of cytokine-induced factors (FIG. S2A). Examples of this mutual dependency for DNA occupancy are shown for several loci (Cd28, 1121, MO, FIGS. 2E and S2B).

The cooperativity of IRF4 and BATF, paired with our finding that cis regions bound by both TFs in TCR stimulation conditions (Th0) exclusively acquire additional strong binding by STAT3, RORγt, c-Maf, and p300 in Th17 cells (FIG. 1B), suggests that IRF4 and BATF function as pioneer factors in nucleating binding of Th17 TFs upon cytokine-stimulated differentiation. Consistent with this, occupancy of IRF4 and BATF is enriched in the center of five-TF pCRMs (FIG. S3A). To investigate this hypothesis directly, we examined chromatin accessibility at regions co-occupied by all five TFs, using Formaldehyde Assisted Isolation of Regulatory Elements sequencing (FAIRE-seq). Deletion of IRF4 or BATF in Th0 or Th17 cells had little effect on regions already accessible in naïve cells, but most regions with inducible FAIRE-seq signal exhibited marked reductions in Irf4^{-/-} and Batf^{-/-} compared to WT cells in both Th17 and Th0 conditions (FIG. S3B). Thus, in the absence of IRF4 and BATF, regions normally bound by all five Th17 TFs are less accessible, providing further evidence that IRF4 and BATF remodel the chromatin landscape, potentially facilitating subsequent recruitment of additional TFs involved in regulating expression of adjacent genes.

Consistent with IRF4 and BATF mediating accessibility, these TFs also globally affect p300 occupancy, which was reduced in IRF4- or BATF-null Th17 cells (FIG. S3C). STAT3 deficiency also reduced p300 binding, but loss of RORγt resulted in a much smaller genome-wide effect (FIG. S3C). The focal influence of RORγt was also reflected in occupancy of IRF4 and STAT3 and the presence of H3K4me2 and H3 Kme3, histone marks associated with active transcription, in RORγt-deficient Th17 cells (FIG. S3D). Strikingly, few genes were dependent on RORγt, as measured by more than 2-fold reduction (and p-value<0.01) in both H3K4me3 at individual locus-linked pCRMs and expression of the respective genes, namely Il17a, Il17f; Il23r, Ccl20, Il1r1, Ltb4r1 that were known; and 2310007L2Rik, Furin, Fam124b, Tmem176a, Tmem176b that represent novel targets. These findings are consistent with the notion that RORγt has a highly specific regulatory footprint relative to initiator TFs that establish broader changes in chromatin remodeling.

### The Th17 Network Reveals Lineage Specification by Combinatorial Regulation

While providing mechanistic insight, TF occupancy does not sufficiently explain target gene regulation. We thus complemented TF ChIP-seq (that finds direct targets) with RNA-seq differential expression analysis of TF wild-type vs. knock-out (KO) Th17 cells (that finds functional targets). ChIP p-values for peaks falling within 10 kb of the gene body were consolidated to a gene-wide p-value, and KO-RNA-seq p-values were calculated based on target gene differential expression. For each data set, p-values were mapped to ranked-based scores from 0 (least significant) to 1 (most significant) and combined such that the integrated ChIP+KO scores ranged from 0 to 2, with the highest scoring genes likely direct and functional targets (see Supplementary Methods). High confidence TF-target interactions (score>1.5; FDR<10%) are visualized in Cytoscape.

The resulting causal network captures known relationships between core TFs, including Rorc activation by STAT3, IRF4, and BATF (Brustle et al., 2007; Schraml et al., 2009; Yang et al., 2007) and further reveals many feed-forward loops that reinforce Rorc expression in response to TCR and cytokine signals (FIG. 3A, box). Notably, there is high interconnectivity among TFs, including positive feedback loops reinforcing expression of initiator TFs BATF, IRF4 and STAT3, and a negative feedback loop (c-Maf to BATF) serving to limit response. Conversely, RORγt does not participate in stabilizing positive feedback relationships with inducing TFs, thus rendering its expression sensitive to changing environmental signals. This is consistent with the need for continuous STAT3 activation (McGeachy et al., 2009) and the plasticity of the Th17 subset when cytokine conditions are altered (Hirota et al., 2011; Lee et al., 2009).

Highly regulated nodes, defined by combinatorial regulation by 4 or 5 core TFs, comprise many genes with critical lineage modulatory and effector activity. These include key signature genes (Il17a, Il17f, Il23r), other relevant cytokines and receptors (Il2, Il9, Lif, Il10, Il1r1, Il21, Il12rb1, Ebi3 (IL-27) Ltb4r1, and Ccr6), and TFs (Rora, Hif1a, Runx1, and Foxo1) (Korn et al., 2009). This indicates that other highly regulated genes, which fall into diverse categories (e.g. ion transport, migration, metabolism, and stress response), have a high likelihood of being novel Th17 regulators or effectors (FIG. 3B).

To assess the regulatory relationships between Th17 TFs, we summarized the activating and repressing regulatory inputs for each gene in the core network in a clustered heat map (FIG. S4A). Notably, initiator TFs BATF, IRF4, and STAT3 regulate the largest number of genes and impose complementary control of shared targets, particularly in activation. This is most striking for highly regulated Th17 genes (FIG. 3C, S4B), and is mirrored by the regulation of similar pathways (FIG. 3D (i)), including helper T cell differentiation and activation, cytokine signaling, metabolism, and oxidative/xenobiotic stress response (some previously attributed to these TFs (Durant et al., 2010; Kwon et al., 2009; Schraml et al., 2009)). Thus, together initiator TFs establish a broad, coherent transcriptional program in Th17 cells.

c-Maf is generally appreciated as an activator of cytokine loci (Ho et al., 1999). Unexpectedly, in Th17 cells it functions mainly as a negative regulator (FIG. 3A, S4A), attenuating the expression of pro-inflammatory loci (e.g. Rora, Runx1, Il1r1, Ccr6, Tnf) and globally repressing genes in pathways regulated by other core TFs (FIG. 3D (i)). Notably c-Maf does positively regulate a few loci, several linked to attenuating inflammation (e.g. Il9, Il10, Lif Ctla4). Together with the recent description of c-Maf as an Il22 repressor (Rutz et al., 2011), the global c-Maf target repertoire identifies an underappreciated general anti-inflammatory role for this TF.

RORγt, the key lineage-specifier, functions as an activator and a repressor within the network (FIG. S4A). Notably, it either reinforces or antagonizes the coherent activation program initiated by IRF4, BATF, and STAT3 (FIG. 3C, S4B). While RORγt positively regulates many genes, it has a strong role at only a small number of key Th17 loci (as defined above; FIG. 3C, S3D; see FIG. S4C for the magnitude of RORγt dependency). As a repressor, RORγt limits target expression, including regulators of metabolism and quiescence (e.g. Il10, Hif1a, Egln3, Foxo1, and Il7r) and alternative lineage fates (Il4ra and Il12rb2) promoted by initiator TFs. In this regard, RORγt acts as a modulator; its repressive activity is poorly correlated with actual expression changes, with its repressed targets often up-regulated in Th17 relative to Th0 cells (FIG. 3C, S4D). Thus, while RORγt serves to reinforce the activation of many genes, it is essential in licensing the expression of a select few loci; elsewhere it functions as a rheostat to tune levels of expression to that of a Th17-specifying program.

Individual Th17 TFs regulate broad cellular functions (FIG. 3D (i)). When network targets were subdivided according to the complexity of inputs (either 1, 2, 3, or 4/5 TF edges), pathway analysis revealed a selective enrichment for genes involved in helper T cell differentiation with increasing number of TF inputs (FIG. 3D, (ii)). In line with this, nodes regulated by 4 or 5 TFs were most enriched for genes highly differentially expressed in Th17 cells (FIG. 3A, compare node color in center vs. periphery). Thus, although lineage TFs orchestrate the expression of genes in similar pathways, lineage specificity is a product of high order combinatorial regulation.

### Data Integration Allows for Discovery of New Th17-Relevant Genes

The ChIP and KO based network is accurate but lacks regulatory information for other TFs with roles in Th17 cells. To learn interactions for those TFs, we integrated into our pipeline two other datasets (FIG. 4A). The four data types are designated: [C] ChIP-seq; [K] RNA-seq KO; [R] 155 helper T cell RNA-seq experiments; and [I] public microarray data spanning 167 immune cell types and conditions (Immunological Genome Project; ImmGen) (Heng and Painter, 2008). In addition to providing regulatory information for new TFs, R and I can provide further support for interactions identified by the other two data types (C, K). Z-scores for putative TF-target regulatory interactions were individually assigned for R and I using our platform, the Inferelator (Greenfield et al., 2010), and converted to ranked-based scores, as above (ranging from 0-1), to facilitate their integration with K and C (Supplementary Methods).

To validate our approach and evaluate complementarity of data sets, we estimated the predictive power of the resulting Th17 networks at recovering 74 previously identified Th17-relevant genes, curated from the literature (Table S1). In addition, given that key Th17 genes were strongly regulated by multiple core TFs (FIG. 3B), we reasoned that ranking genes based on their summed scores over all five core TFs would enrich for relevant target genes. We used two performance metrics, the areas under precision recall (aucPR) and under receiver operator curves (aucROC) that together provide a balance between estimating the sensitivity (aucROC) and accuracy (aucPR) of top ranked predictions. Regardless of the metric used, for each data combination, summed TF target predictions (FIG. 4B; bar) better identified Th17-relevant genes than targets for individual TFs (FIG. 4B; points in bar), highlighting the predictive power of leveraging combinatorial regulation (also FIG. S5A). Moreover, combining across data types resulted in higher quality networks (note the performance increase with data integration, FIG. 4B, S5A). This is true for both K+C that produces a smaller (core-TF centered) but accurate network, and for R+I that generates a more comprehensive (more than 170 differentially expressed TFs) but less accurate model. Thus, combining data as in K+C+R+I provides additional network information without compromising the K+C network accuracy, leading to a three-fold performance boost at finding relevant Th17 genes over the conventional differential expression list (FIG. 4B, dotted line). Moreover, the non-parametric ranked-based scheme is better suited for combining data than an alternative parametric Fisher method for combining p-values (FIG. S5C, D).

We further characterized the predictions of the top performing KCRI network using gene set enrichment analysis (GSEA; FIG. 4C). Literature-curated genes were highly enriched as top predictions (60 of 74 total), ranking many more putative Th17-relevant genes with comparable KCRI scores (1,328 of ˜22,000 total).

The wealth of GWAS information for diseases in which Th17 cells are implicated allowed us to judge the relevance of our mouse Th17 network for human disease (Hindorff et al., 2012). We found that loci linked to SNPs associated with ulcerative colitis (UC), Crohn's disease (CD), multiple sclerosis, psoriasis and rheumatoid arthritis were significantly enriched in the core (KCRI) Th17 network (FIGS. 4D, E, S5B). This is in striking contrast to diseases that have enrichment scores expected by chance (Alzheimer's disease and schizophrenia) and other inflammatory diseases in which Th17 cell involvement is not established (e.g. type 2 diabetes and systemic lupus erythematosus (SLE)). Notably, for those diseases with strong links to Th17 function, GWAS associated genes are more highly regulated by the core Th17 TFs (FIG. 4E), and TF sum scores (FIG. 4D; bars) better recovered GWAS loci relative to individual TFs (FIG. 4D; points). This strongly supports the notion that synergy between the five core Th17 TFs is not limited to specification in mouse models, but is also relevant for function of the Th17 lineage in human disease.

### Network Analysis Identifies Novel Modulators of the Th17 Program

Our validations revealed that highly regulated targets of the KCRI network can be exploited to identify new Th17-relevant effector genes. However, early response regulators that function upstream of or in parallel with core TFs may not be captured (FIG. 3A). To address this, we used an independent Inferelator-derived ImmGen network (I) to predict new TFs that demonstrate significant target overlap with the five core TFs (KC network; see Supplementary Methods); a similar method was proven successful (Carro et al., 2010). Accordingly, 26 new candidate Th17 TFs were prioritized that either 1) were top scoring KCRI network-regulated genes (90^{th} percentile), or 2) showed significant overlap between their predicted targets and targets of the five core TFs. The latter TF enrichment analysis identified all core TFs as top hits and several known Th17 regulators (see full list Table S2), including RORα, AHR, RBPJ, and TBX21 (Alam et al., 2010; Veldhoen et al., 2008; Yang et al., 2008).

To assess the effects of prioritized TFs on Th17 cell differentiation, we performed gain- and loss-of-function experiments. Among the 16 TFs overexpressed in CD4⁺ T cells by retroviral transduction, several had a significant effect on the percentage of IL-17A⁺ cells generated, including Ets factor Etv6, Ncoa2, Smad3, Hif1a, Skil, and Trib3 (FIG. S6A, B). Most striking was the AP-1 family member Fos12, the top predicted factor in the TF enrichment analysis (Table S2), whose overexpression significantly reduced the number of IL-17A-producing cells.

In a complementary approach, we performed siRNA-mediated knock-down (KD) experiments for 14 candidate TFs with siRNA pools electroporated into activated CD4⁺ T cells subjected to Th17 polarizing conditions. The reductions in target TF mRNAs were similar to those observed with siRorc, which effectively reduced the amount of RORγt (FIG0 S6C, D), its known targets (Il17a/f, Il23r, Il22), and Th17 differentiation (FIGS. 5A,B, S6C). Strikingly, six TF KDs significantly altered IL-17A production relative to a non-targeting control, without affecting the concomitant generation of Foxp3⁺iTreg cells. These included Etv6, Nfatc2, Bcl11b, Crem, and regulators of chromatin remodeling (Satb1, Kdm6b (Jmjd3)) (FIGS. 5A, 5B). Thus, the network method prioritized and made accurate predictions about genes that influence expression of IL-17A, a key component of the Th17 phenotype.

To assess factor influence on the broad Th17 program, we performed RNA-seq of TF KD cultures. Global pathway analysis for TF-dependent genes identified distinct factor-related pathways, yet showed a striking convergence in enrichment for genes involved in T helper cell differentiation/function for all factors, except Sirt2 and a non-TF control, Ccr6 (FIG. 5C). Indeed, TF-dependent loci comprise a variety of helper T cell effector genes (Il22, Il1r1, Il23r, Il10, Il24, Il9, Ccl20), and lineage-specializing genes (Il4, Ifng, Gata3, Foxp3, Tbx21) (FIG. 5D). Of particular interest, Bcl11b influenced the expression of a broad set of helper T cell-modulatory genes, suggesting a key regulatory role in subset diversification. Similarly, Jmjd3, a lysine K27 demethylase and known T-bet partner (Miller et al., 2010), displayed a marked influence on the activation of multiple Th17-expressed cytokines, suggesting that it may partner with core Th17 TFs. Indeed, Jmjd3 shares many direct targets (KD+C) with RORγt and STAT3 (FIG. S6E). Taken together, the results show that the network model identifies many novel candidate modulators of the Th17 program.

### Fos12 is a Core Component of the Th17 Specification Program

The AP-1 family TF Fos12 was the highest-ranking candidate to co-regulate targets with core TFs (Table S2). We interrogated its role in helper T cell differentiation and function using mice with conditional deletion of Fos/2 in T cells (Fos12^{fl/fl} CD4-Cre). Fos12-deficient CD4⁺ T cells could be polarized in vitro into Th1, Th2, Th17 or iTreg cells, but, notably, cytokine production was dysregulated (FIG. 6A, S7A). Fos12-null Th17 cultures were markedly increased for IL-17A-producing and atypical Foxp3⁺IL-17A⁺ cells. They also had low-level derepression of Il17a in Th1 and Th2 cells, consistent with Fos12 function as an Il17a repressor (FIGS. S6A,B, S7A). Fos12-deficiency also enabled IFNγ production in Th17 and Th2 cultures, particularly when Th17 cells were subsequently exposed to Th1-skewing conditions (FIG. S7A, B).

We next examined the role of Fos12 in the Th17-dependent disease model experimental autoimmune encephalomyelitis (EAE), which mimics the CNS pathology in multiple sclerosis. Fos12^{fl/fl} CD4-Cre mice had significantly attenuated disease severity compared to wild-type controls (FIG. 6B). Analysis of spinal cord infiltrates at 21 days post immunization revealed reduced CD4⁺ T cells, but similar percentages of IL-17A, IFNγ, and GM-CSF producers, in mutant mice. Strikingly, the Fos12-deficient cytokine-producing T-helper cells also expressed the TF Foxp3, which specifies the Treg program (FIG. 6 B, C). Consistent with our in vitro observations (FIG. 6A), these findings suggest Fos12 as a key regulator of T-helper lineage plasticity, particularly under inflammatory conditions.

To gain insight into Fos12 function we identified its direct targets using K+C analysis. Consistent with previous studies, Fos12 both activates and represses target loci (Wagner and Efer1, 2005). It attenuates expression of Th17 signature genes in addition to Il17a (Il17f, Ccl20, Ccr6, Il1r1, Batf) and of Th1-regulatory loci (Tbx21, Il18r1, Il18rap, and Il2), suggesting a role in controlling inflammatory responses and preventing Th1 specification (FIG. 6D). Conversely, Fos12 also promotes the expression of genes that drive Th17 maintenance and survival (Il6ra, Il-23r, Il12rb1, il7r, Il21) and helper T cell diversification or function (Il4ra, Il12rb2, Il2ra, Il10ra, Ltb4r1, Smad3, Hif1a) (FIG. 6D). These loci are also targeted by the five core TFs, indicating that Fos12 modulates the lineage identity and functional programs regulated by core Th17 TFs.

In light of the dominant regulation by AP-1 factors in the Th17 lineage, it was interesting to observe a high degree of overlap in occupancy by Fos12 and BATF (FIG. 6E), suggestive of an antagonistic relationship between them. This may be mediated by direct competition for the same binding sites (FIG. S7C), and enhanced by direct transcriptional repression of Batf by Fos12 (FIG. S7D). Thus, as predicted, Fos12 is a highly interconnected component of the core Th17 specification program. This is in contrast to Hif1α, a recently identified regulator of Th17 cells (Dang et al., 2011), that was not predicted to share a significant number of targets with core TFs (Table S3) and is not as interconnected as Fos12 (FIG. 6D).

### Visualization and Exploration of the Extended KCRI Th17 Network

The final KCRI network comprises 173 differentially expressed TFs controlling 3,679 genes with approximately 19,000 interactions. The subset of this network surrounding regulators based on ChIP-seq and KO data is more accurate but limited (this network has 7 TFs controlling 2218 genes, with 4237 edges). To facilitate interrogation of the Th17 transcriptional network by the scientific community, we provide access to the primary data (see Table S3, Supplemental Data), the networks (KC and KCRI), and analysis tools via the worldwide web at th17 bio nyu edu.

### REFERENCES

Alam, M. S., Maekawa, Y., Kitamura, A., Tanigaki, K., Yoshimoto, T., Kishihara, K., and Yasutomo, K. (2010). Notch signaling drives IL-22 secretion in CD4+ T cells by stimulating the aryl hydrocarbon receptor. Proc Natl Acad Sci USA 107, 5943-5948.
Bauquet, A. T., Jin, H., Paterson, A. M., Mitsdoerffer, M., Ho, I. C., Sharpe, A. H., and Kuchroo, V. K. (2009). The costimulatory molecule ICOS regulates the expression of c-Maf and IL-21 in the development of follicular T helper cells and TH-17 cells. Nat Immunol 10, 167-175.
Bonneau, R., Facciotti, M. T., Reiss, D. J., Schmid, A. K., Pan, M., Kaur, A., Thorsson, V., Shannon, P., Johnson, M. H., Bare, J. C., et al. (2007). A predictive model for transcriptional control of physiology in a free living cell. Cell 131, 1354-1365.
Brustle, A., Heink, S., Huber, M., Rosenplanter, C., Stadelmann, C., Yu, P., Arpaia, E., Mak, T. W., Kamradt, T., and Lohoff, M. (2007). The development of inflammatory T(H)-17 cells requires interferon-regulatory factor 4. Nat Immunol 8, 958-966.
Califano, A., Butte, A. J., Friend, S., Ideker, T., and Schadt, E. (2012). Leveraging models of cell regulation and GWAS data in integrative network-based association studies. Nat Genet. 44, 841-847.
Carro, M. S., Lim, W. K., Alvarez, M. J., Bollo, R. J., Zhao, X., Snyder, E. Y., Sulman, E. P., Anne, S. L., Doetsch, F., Colman, H., et al. (2010). The transcriptional network for mesenchymal transformation of brain tumours. Nature 463, 318-325.
Codarri, L., Gyulveszi, G., Tosevski, V., Hesske, L., Fontana, A., Magnenat, L., Suter, T., and Becher, B. (2011). RORgammat drives production of the cytokine GM-CSF in helper T cells, which is essential for the effector phase of autoimmune neuroinflammation. Nat Immunol 12, 560-567.
Crotty, S. (2011). Follicular helper CD4 T cells (TFH). Annu Rev Immunol 29, 621-663.
Dang, E. V., Barbi, J., Yang, H. Y., Jinasena, D., Yu, H., Zheng, Y., Bordman, Z., Fu, J., Kim, Y., Yen, H. R., et al. (2011). Control of T(H)17/T(reg) balance by hypoxia-inducible factor 1. Cell 146, 772-784.
Durant, L., Watford, W. T., Ramos, H. L., Laurence, A., Vahedi, G., Wei, L., Takahashi, H., Sun, H. W., Kanno, Y., Powrie, F., et al. (2010). Diverse targets of the transcription factor STAT3 contribute to T cell pathogenicity and homeostasis. Immunity 32, 605-615.
Eisenbeis, C. F., Singh, H., and Storb, U. (1995). Pip, a novel IRF family member, is a lymphoid-specific, PU.1-dependent transcriptional activator. Genes Dev 9, 1377-1387.
Ernst, J., Vainas, 0., Harbison, C. T., Simon, I., and Bar-Joseph, Z. (2007). Reconstructing dynamic regulatory maps. Mol Syst Biol 3, 74.
Faith, J. J., Hayete, B., Thaden, J. T., Mogno, I., Wierzbowski, J., Cottarel, G., Kasif, S., Collins, J. J., and Gardner, T. S. (2007). Large-scale mapping and validation of Escherichia coli transcriptional regulation from a compendium of expression profiles. PLoS Biol 5, e8.
Greenfield, A., Madar, A., Ostrer, H., and Bonneau, R. (2010). DREAM4: Combining genetic and dynamic information to identify biological networks and dynamical models. PLoS One 5, e13397.
Heng, T. S., and Painter, M. W. (2008). The Immunological Genome Project: networks of gene expression in immune cells. Nat Immunol 9, 1091-1094.
Hindorff, L. A., MacArthur, J., Wise, A., Junkins, R A., Hall, P. N., Klemm, A. K., and Manolio, T. A. (Accessed Feb. 29, 2012). A Catalog of Published Genome-Wide Association Studies. In Available at: wwwgenomegov/gwastudies.
Hirota, K., Duarte, J. H., Veldhoen, M., Hornsby, E., L₁, Y., Cua, D. J., Ahlfors, H., Wilhelm, C., Tolaini, M., Menzel, U., et al. (2011). Fate mapping of IL-17-producing T cells in inflammatory responses. Nat Immunol 12, 255-263.
Ho, I. C., Kim, J. I., Szabo, S. J., and Glimcher, L. H. (1999). Tissue-specific regulation of cytokine gene expression. Cold Spring Harb Symp Quant Biol 64, 573-584.
Ise, W., Kohyama, M., Schraml, B. U., Zhang, T., Schwer, B., Basu, U., Alt, F. W., Tang, J., Oltz, E. M., Murphy, T. L., et al. (2011). The transcription factor BATF controls the global regulators of class-switch recombination in both B cells and T cells. Nat Immunol 12, 536-543.
Ivanov, II, McKenzie, B. S., Zhou, L., Tadokoro, C. E., Lepelley, A., Lafulle, J. J., Cua, D. J., and Littman, D. R. (2006). The orphan nuclear receptor RORgammat directs the differentiation program of proinflammatory IL-17+ T helper cells. Cell 126, 1121-1133.
Korn, T., Bettelli, E., Oukka, M., and Kuchroo, V. K. (2009). IL-17 and Th17 Cells. Annu Rev Immunol.
Kwon, H., Thierry-Mieg, D., Thierry-Mieg, J., Kim, H. P., Oh, J., Tunyaplin, C., Carotta, S.,
Donovan, C. E., Goldman, M. L., Tailor, P., et al. (2009). Analysis of interleukin-21-induced Prdml gene regulation reveals functional cooperation of STAT3 and IRF4 transcription factors. Immunity 31, 941-952.
Lee, Y. K., Turner, H., Maynard, C. L., Oliver, J. R., Chen, D., Elson, C. O., and Weaver, C. T. (2009). Late developmental plasticity in the T helper 17 lineage. Immunity 30, 92-107.
Leppkes, M., Becker, C., Ivanov, II, Hirth, S., Wirtz, S., Neufert, C., Pouly, S., Murphy, A. J., Valenzuela, D. M., Yancopoulos, G. D., et al. (2009). RORgamma-expressing Th17 cells induce murine chronic intestinal inflammation via redundant effects of IL-17A and IL-17F. Gastroenterology 136, 257-267.
Manel, N., Unutmaz, D., and Littman, D. R. (2008). The differentiation of human T(H)-17 cells requires transforming growth factor-beta and induction of the nuclear receptor RORgammat. Nat Immunol 9, 641-649.
Marbach, D., Roy, S., Ay, F., Meyer, P. E., Candeias, R., Kahveci, T., Bristow, C. A., and Kellis, M. (2012). Predictive regulatory models in Drosophila melanogaster by integrative inference of transcriptional networks. Genome Res.
Mattick, J. S., Taft, R. J., and Faulkner, G. J. (2010). A global view of genomic information—moving beyond the gene and the master regulator. Trends Genet. 26, 21-28.
McGeachy, M. J., Chen, Y., Tato, C. M., Laurence, A., Joyce-Shaikh, B., Blumenschein, W. M., McClanahan, T. K., O'Shea, J. J., and Cua, D. J. (2009). The interleukin 23 receptor is essential for the terminal differentiation of interleukin 17-producing effector T helper cells in vivo. Nat Immunol 10, 314-324.
Miller, S. A., Mohn, S. E., and Weinmann, A. S. (2010). Jmjd3 and UTX play a demethylase-independent role in chromatin remodeling to regulate T-box family member-dependent gene expression. Mol Cell 40, 594-605.
Novershtern, N., Subramanian, A., Lawton, L. N., Mak, R. H., Haining, W. N., McConkey, M. E., Habib, N., Yosef, N., Chang, C. Y., Shay, T., et al. (2011). Densely interconnected transcriptional circuits control cell states in human hematopoiesis. Cell 144, 296-309.
Okamoto, K., Iwai, Y., Oh-Hora, M., Yamamoto, M., Mono, T., Aoki, K., Ohya, K., Jetten, A M., Akira, S., Muta, T., et al. (2010). IkappaBzeta regulates T(H)17 development by cooperating with ROR nuclear receptors. Nature 464, 1381-1385.
Rengarajan, J., Mowen, K. A., McBride, K. D., Smith, E. D., Singh, H., and Glimcher, L. H. (2002). Interferon regulatory factor 4 (IRF4) interacts with NFATc2 to modulate interleukin 4 gene expression. J Exp Med 195, 1003-1012.
Rutz, S., Noubade, R., Eidenschenk, C., Ota, N., Zeng, W., Zheng, Y., Hackney, J., Ding, J., Singh, H., and Ouyang, W. (2011). Transcription factor c-Maf mediates the TGF-beta-dependent suppression of IL-22 production in T(H)17 cells. Nat Immunol 12, 1238-1245.
Schraml, B. U., Hildner, K., Ise, W., Lee, W. L., Smith, W. A., Solomon, B., Sahota, G., Sim, J., Mukasa, R., Cemerski, S., et al. (2009). The AP-1 transcription factor Batf controls T(H)17 differentiation. Nature 460, 405-409.
Veldhoen, M., Hirota, K., Westendorf, A M., Buer, J., Dumoutier, L., Renauld, J. C., and Stockinger, B. (2008). The aryl hydrocarbon receptor links TH17-cell-mediated autoimmunity to environmental toxins. Nature 453, 106-109.
Visel, A., Blow, M. J., L₁, Z., Zhang, T., Akiyama, J. A., Holt, A., Plajzer-Frick, I., Shoukry, M., Wright, C., Chen, F., et al. (2009). ChIP-seq accurately predicts tissue-specific activity of enhancers. Nature 457, 854-858.
Voo, K. S., Wang, Y. H., Santori, F. R., Boggiano, C., Wang, Y. H., Arima, K., Boyer, L., Hanabuchi, S., Khalili, J., Marinova, E., et al. (2009). Identification of IL-17-producing FOXP3+regulatory T cells in humans. Proc Natl Acad Sci USA 106, 4793-4798.
Wagner, E. F., and Eferl, R. (2005). Fos/AP-1 proteins in bone and the immune system. Immunol Rev 208, 126-140.
Wei, G., Wei, L., Zhu, J., Zang, C., Hu-L₁, J., Yao, Z., Cui, K., Kanno, Y., Roh, T. Y., Watford, W. T., et al. (2009). Global mapping of H3K4me3 and H3K27me3 reveals specificity and plasticity in lineage fate determination of differentiating CD4+ T cells. Immunity 30, 155-167.
Yang, X. O., Panopoulos, A D., Nurieva, R., Chang, S. H., Wang, D., Watowich, S. S., and Dong, C. (2007). STAT3 regulates cytokine-mediated generation of inflammatory helper T cells. J Biol Chem 282, 9358-9363.
Yang, X. O., Pappu, B. P., Nurieva, R., Akimzhanov, A., Kang, H. S., Chung, Y., Ma, L., Shah, B., Panopoulos, A. D., Schluns, K. S., et al. (2008). T helper 17 lineage differentiation is programmed by orphan nuclear receptors ROR alpha and ROR gamma. Immunity 28, 29-39.
Zaret, K. S., and Carroll, J. S. (2011). Pioneer transcription factors: establishing competence for gene expression. Genes Dev 25, 2227-2241.
Zhang, F., Meng, G., and Strober, W. (2008). Interactions among the transcription factors Runx1, RORgammat and Foxp3 regulate the differentiation of interleukin 17-producing T cells. Nat Immunol 9, 1297-1306.
Zhu, J., Yamane, H., and Paul, W. E. (2010). Differentiation of effector CD4 T cell populations. Annu Rev Immunol 28, 445-489.

### SUPPLEMENTAL REFERENCES

Anders, S., and Huber, W. (2010). Differential expression analysis for sequence count data. Genome biology 11, R106.
Bonneau, R., Facciotti, M. T., Reiss, D. J., Schmid, A K., Pan, M., Kaur, A., Thorsson, V.,
Shannon, P., Johnson, M. H., Bare, J. C., et al. (2007). A predictive model for transcriptional control of physiology in a free living cell. Cell 131, 1354-1365.
Bonneau, R., Reiss, D. J., Shannon, P., Facciotti, M., Hood, L., Baliga, N. S., and Thorsson, V. (2006). The Inferelator: an algorithm for learning parsimonious regulatory networks from systems-biology data sets de novo. Genome biology 7, R36.
Boyer, L. A., Lee, T. I., Cole, M. F., Johnstone, S. E., Levine, S. S., Zucker, J. P., Guenther, M. G., Kumar, R. M., Murray, H. L., Jenner, R. G., et al. (2005). Core transcriptional regulatory circuitry in human embryonic stem cells. Cell 122, 947-956.
Carro, M. S., Lim, W. K., Alvarez, M. J., Bollo, R. J., Zhao, X., Snyder, E. Y., Sulman, E. P., Anne, S. L., Doetsch, F., Colman, H., et al. (2010). The transcriptional network for mesenchymal transformation of brain tumours. Nature 463, 318-325.
Chen, X., Xu, H., Yuan, P., Fang, F., Huss, M., Vega, V. B., Wong, E., Orlov, Y. L., Zhang, W., Jiang, J., et al. (2008). Integration of external signaling pathways with the core transcriptional network in embryonic stem cells. Cell /33, 1106-1117.
Ebert, G., Marmon, S., Sunshine, M. J., Rennert, P. D., Choi, Y., and Littman, D. R. (2004). An essential function for the nuclear receptor RORgamma(t) in the generation of fetal lymphoid tissue inducer cells. Nat Immunol 5, 64-73.
Faith, J. J., Hayete, B., Thaden, J. T., Mogno, I., Wierzbowski, J., Cottarel, G., Kasif, S., Collins, J. J., and Gardner, T. S. (2007). Large-scale mapping and validation of Escherichia coli transcriptional regulation from a compendium of expression profiles. PLoS biology 5, e8.
Fisher, R. A. (1925). Statistical Methods for Research Workers (Edinburgh: Oliver and Boyd).
Gertz, J., Varley, K. E., Davis, N. S., Baas, B. J., Goryshin, I. Y., Vaidyanathan, R., Kuersten, S., and Myers, R. M. (2012). Transposase mediated construction of RNA-seq libraries. Genome Res 22, 134-141.
Gilchrist, M., Thorsson, V., L₁, B., Rust, A G., Korb, M., Roach, J. C., Kennedy, K., Hai, T., Bolouri, H., and Aderem, A. (2006). Systems biology approaches identify ATF3 as a negative regulator of Toll-like receptor 4. Nature 441, 173-178.
Goecks, J., Nekrutenko, A., and Taylor, J. (2010). Galaxy: a comprehensive approach for supporting accessible, reproducible, and transparent computational research in the life sciences. Genome Biol 11, R86.
Heinz, S., Benner, C., Spann, N., Bertolino, E., Lin, Y. C., Laslo, P., Cheng, J. X., Murre, C., Singh, H., and Glass, C. K. (2010). Simple combinations of lineage-determining transcription factors prime cis-regulatory elements required for macrophage and B cell identities. Mol Cell 38, 576-589.
Heng, T. S., and Painter, M. W. (2008). The Immunological Genome Project: networks of gene expression in immune cells. Nature immunology 9, 1091-1094.
Ivanov, II, McKenzie, B. S., Zhou, L., Tadokoro, C. E., Lepelley, A., Lafulle, J. J., Cua, D. J., and Littman, D. R. (2006). The orphan nuclear receptor RORgammat directs the differentiation program of proinflammatory IL-17+ T helper cells. Cell 126, 1121-1133.
Johnson, D. S., Mortazavi, A., Myers, R. M., and Wold, B. (2007). Genome-wide mapping of in vivo protein-DNA interactions. Science 316, 1497-1502.
Karreth, F., Hoebertz, A., Scheuch, H., Eferl, R., and Wagner, E. F. (2004). The AP1 transcription factor Fra2 is required for efficient cartilage development. Development 131, 5717-5725.
Kirigin, F. F., Lindstedt, K., Sellars, M., Ciofani, M., Low, S. L., Jones, L., Bell, F., Pauli, F., Bonneau, R., Myers, R. M., et al. (2012). Dynamic microRNA gene transcription and processing during T cell development. J Immunol 188, 3257-3267.
Klein, U., Casola, S., Cattoretti, G., Shen, Q., Lia, M., Mo, T., Ludwig, T., Rajewsky, K., and Dalla-Favera, R. (2006). Transcription factor IRF4 controls plasma cell differentiation and class-switch recombination. Nat Immunol 7, 773-782.
Langmead, B., Trapnell, C., Pop, M., and Salzberg, S. L. (2009). Ultrafast and memory-efficient alignment of short DNA sequences to the human genome. Genome Biol 10, R25.
Lee, C. K., Raz, R., Gimeno, R., Gertner, R., Wistinghausen, B., Takeshita, K., DePinho, R. A., and Levy, D. E. (2002). STAT3 is a negative regulator of granulopoiesis but is not required for G-CSF-dependent differentiation. Immunity 17, 63-72.
Lefebvre, C., Rajbhandari, P., Alvarez, M. J., Bandaru, P., Lim, W. K., Sato, M., Wang, K., Sumazin, P., Kustagi, M., Bisikirska, B. C., et al. (2010). A human B-cell interactome identifies MYB and FOXM1 as master regulators of proliferation in germinal centers. Mol Syst Biol 6, 377.
Machanick, P., and Bailey, T. L. (2011). MEME-ChIP: motif analysis of large DNA datasets. Bioinformatics 27, 1696-1697.
Madar, A., Greenfield, A., Ostrer, H., Vanden-Eijnden, E., and Bonneau, R. (2009). The Inferelator 2.0: a scalable framework for reconstruction of dynamic regulatory network models. Conference proceedings: Annual International Conference of the IEEE Engineering in Medicine and Biology Society IEEE Engineering in Medicine and Biology Society Conference 2009, 5448-5451.
Madar, A., Greenfield, A., Vanden-Eijnden, E., and Bonneau, R. (2010). DREAM3: network inference using dynamic context likelihood of relatedness and the inferelator. PloS one 5, e9803.
Marbach, D., Costello, J. C., Kuffner, R., Vega, N. M., Prill, R. J., Camacho, D. M., Allison, K R., Aderhold, A., Allison, K. R., Bonneau, R., et al. (2012a). Wisdom of crowds for robust gene network inference. Nature methods. Marbach, D., Roy, S., Ay, F., Meyer, P. E., Candeias, R., Kahveci, T., Bristow, C. A., and Kellis, M. (2012b). Predictive regulatory models in Drosophila melanogaster by integrative inference of transcriptional networks. Genome research.
Marson, A., Levine, S. S., Cole, M. F., Frampton, G. M., Brambrink, T., Johnstone, S., Guenther, M. G., Johnston, W. K., Wernig, M., Newman, J., et al. (2008). Connecting microRNA genes to the core transcriptional regulatory circuitry of embryonic stem cells. Cell 134, 521-533.
Morita, S., Kojima, T., and Kitamura, T. (2000). Plat-E: an efficient and stable system for transient packaging of retroviruses. Gene Ther 7, 1063-1066.
Mortazavi, A., Williams, B. A., McCue, K., Schaeffer, L., and Wold, B. (2008). Mapping and quantifying mammalian transcriptomes by RNA-Seq. Nat. Methods 5, 621-628.
Ouyang, Z., Zhou, Q., and Wong, W. H. (2009). ChIP-Seq of transcription factors predicts absolute and differential gene expression in embryonic stem cells. Proceedings of the National Academy of Sciences of the United States of America 106, 21521-21526.
Park, H., L₁, Z., Yang, X. O., Chang, S. H., Nurieva, R., Wang, Y. H., Wang, Y., Hood, L., Zhu, Z., Tian, Q., et al. (2005). A distinct lineage of CD4 T cells regulates tissue inflammation by producing interleukin 17. Nat Immunol 6, 1133-1141.
Prill, R. J., Marbach, D., Saez-Rodriguez, J., Sorger, P. K., Alexopoulos, L. G., Xue, X., Clarke, N. D., Altan-Bonnet, G., and Stolovitzky, G. (2010). Towards a rigorous assessment of systems biology models: the DREAM3 challenges. PloS one 5, e9202.
Reddy, T. E., Gertz, J., Pauli, F., Kucera, K. S., Varley, K. E., Newberry, K. M., Marinov, G. K., Mortazavi, A., Williams, B. A., Song, L., et al. (2012). Effects of sequence variation on differential allelic transcription factor occupancy and gene expression. Genome Res 22, 860-869.
Ryan, H. E., Poloni, M., McNulty, W., Elson, D., Gassmann, M., Arbeit, J. M., and Johnson, R. S. (2000). Hypoxia-inducible factor-1 alpha is a positive factor in solid tumor growth. Cancer Res 60, 4010-4015.
Schraml, B. U., Hildner, K., Ise, W., Lee, W. L., Smith, W. A., Solomon, B., Sahota, G., Sim, J., Mukasa, R., Cemerski, S., et al. (2009). The AP-1 transcription factor Batf controls T(H)17 differentiation. Nature 460, 405-409.
Simon, J. M., Giresi, P. G., Davis, I. J., and Lieb, J. D. (2012). Using formaldehyde-assisted isolation of regulatory elements (FAIRE) to isolate active regulatory DNA. Nat Protoc 7, 256-267.
Subramanian, A., Tamayo, P., Mootha, V. K., Mukherjee, S., Ebert, B. L., Gillette, M. A., Paulovich, A., Pomeroy, S. L., Golub, T. R., Lander, E. S., et al. (2005). Gene set enrichment analysis: a knowledge-based approach for interpreting genome-wide expression profiles. Proc Natl Acad Sci USA 102, 15545-15550.
Trapnell, C., Pachter, L., and Salzberg, S. L. (2009). TopHat: discovering splice junctions with RNA-Seq. Bioinformatics 25, 1105-1111.
Trapnell, C., Williams, B. A., Pertea, G., Mortazavi, A., Kwan, G., van Baren, M. J., Salzberg, S. L., Wold, B. J., and Pachter, L. (2010). Transcript assembly and quantification by RNA-Seq reveals unannotated transcripts and isoform switching during cell differentiation. Nat Biotechnol 28, 511-515.
Wang, X., Zhang, Y., Yang, X. O., Nurieva, R. I., Chang, S. H., Ojeda, S. S., Kang, H. S., Schluns, K. S., Gui, J., Jetten, A. M., et al. (2012). Transcription of 1117 and 1117f is controlled by conserved noncoding sequence 2. Immunity 36, 23-31.
Wende, H., Lechner, S. G., Cheret, C., Bourane, S., Kolanczyk, M. E., Pattyn, A., Reuter, K., Munier, F. L., Carroll, P., Lewin, G. R., et al. (2012). The transcription factor c-Maf controls touch receptor development and function. Science 335, 1373-1376.
Ye, T., Krebs, A R., Choukrallah, M. A., Keime, C., Plewniak, F., Davidson, I., and Tora, L. (2011). seqMINER: an integrated ChIP-seq data interpretation platform. Nucleic Acids Res 39, e35.
Zhang, Y., Liu, T., Meyer, C. A., Eeckhoute, J., Johnson, D. S., Bernstein, B. E., Nusbaum, C., Myers, R. M., Brown, M., L₁, W., et al. (2008). Model-based analysis of ChIP-Seq (MACS). Genome biology 9, R137.
Zhou, X., Sumazin, P., Rajbhandari, P., and Califano, A. (2010). A systems biology approach to transcription factor binding site prediction. PloS one 5, e9878.
Zou, H., and Hastie, T. (2005). Regularization and variable selection via the elastic net. J Roy Stat Soc B 67, 301-320.

What is claimed is:

1. A method for screening to identify a modulator of a Th17 regulatory network (TRN) protein, the method comprising: contacting a population of naive CD4⁺ T cells polarized under Th17 conditions with at least one candidate agent and assessing at least one transcriptional readout of TRN protein activity in the presence or absence of the at least one candidate agent, wherein a change in level of the at least one transcriptional readout indicates that the least one candidate agent is a TRN protein modulator.

2. The method of claim 1, wherein the change detected in the presence of the candidate modulator agent is a reduction in the at least one transcriptional readout, thereby identifying the candidate modulator agent as an inhibitor of the TRN protein activity.

3. The method of claim 1, wherein the change detected in the presence of the candidate modulator agent is an increase in the at least one transcriptional readout, thereby identifying the candidate modulator agent as an enhancer of the TRN protein activity.

4. The method of claim 1, wherein the at least one transcriptional readout is detecting expression of at least one of a transcription factor-dependent loci selected from the group consisting of Il17a, Il17f, Il22, Il1r1, Il23r, Il10, Il24, Il9, Ccl20, Il4, Ifng, Gata3, Foxp3, and Tbx21.

5. The method of claim 1, wherein the at least one transcriptional readout is detecting expression of at least one of a lineage-specializing gene selected from the group consisting of Rorc, Gata3, Foxp3, Tbx21, 114, and Ifng.

6. The method of claim 1, wherein the at least one transcriptional readout is detecting expression of a Th17 cell cytokine.

7. The method of claim 6, wherein the Th17 cell cytokine is IL17A, IL17F, IL22, or IL21.

8. The method of claim 1, wherein the at least one transcriptional readout is an exogenous marker whose expression is regulated by the TRN protein activity.

9. The method of claim 1, wherein the candidate modulator agent is a small organic molecule, a protein, a peptide, a nucleic acid, a carbohydrate, or an antibody.

10. The method of claim 1, wherein the TRN protein is selected from the group listed in Table S4.

11. The method of claim 1, wherein the TRN protein is selected from the group listed in Table 1.

12. The method of claim 1, wherein the TRN protein is selected from the group listed in Table 2.

13. The method of claim 1, wherein the TRN protein is selected from the group consisting of Fos12, Etv6, Nfatc2, Crem, Satb1, Bcl11b, Jmjd3, Ncoa2, Skil, and Trib3.

14. The method of claim 1, wherein the TRN protein is Etv6, Nfatc2, Bcl11b, Crem, Satb1, or Kdm6b (Jmjd3).

15. The method of claim 1, wherein the TRN protein is Fos12.

16. The method of claim 1, wherein the TRN protein is JMJD3.

17. A method for treating a subject afflicted with an inflammatory condition or autoimmune disease associated with Th17 cell mediated pathology, the method comprising administering GSK-J1 to the subject, wherein the GSK-J1 reduces Th17 cell activity in the subject and thereby treats the subject.

18. The method of claim 17, wherein the inflammatory condition or autoimmune disease is Crohn's disease, ulcerative colitis, multiple sclerosis, rheumatoid arthritis, or psoriasis.

19. The method of claim 17, wherein the mammalian subject is a human.

20. A method for screening to identify a modulator of Th17 cell specification, the method comprising contacting a population of naïve CD4⁺ T cells polarized under Th17 conditions with at least one candidate agent and assessing activity of at least one Th17 regulatory network (TRN) protein in the presence or absence of the at least one candidate agent, wherein a change in activity level of the at least one TRN protein identifies the at least one candidate agent as a modulator of Th17 cell specification.


SEQUENCE LISTING (4 sequences)

SEQ ID NO: 1 - DNA, 2362 bp, Mus musculus
ggtagtgactcatctcgggcagagcgcaggggctcgaagcgagacagtgagcggcgctcggcagggacagagagagagaaagagagcgagcgcaagaaagagggggagagaggggagggcgaagcgaggcgagaagcgcgaggcttggcaggtcctggagccagcacggggtgcaggtcactgcgacgcggagccacgtccctctcagcaccgctcagcctagaatcccacgggcgctgtcccaccggcgcgatcggggccgctggaccggcgctcaccgccttgtcctaagtcaagtgtccgagtcgccgggaaactcaggcggcgagttggccacaggaagtttattctcgggctccttttctaaaaggaggaaacagaagtttctcccaaccggcagcttttctttttcgcctttttggcgccctgagagatcggggagggagtcccccagagctgccaagcgacaccccgcggggctcctagtctttttgcgttgccccatttttctccagccccgtgggtcccggcagagctccggctgcgcgcgggggcggaagggcgcgcgcaggggagggaccgagggacgcgccgactttttagagggagggactcggtggacaaccggttccgcggagcttgcagagcagaaacgaagtgcggtgcgcggcgatcgggggggtcacttcccgagtgtcgctgcctcctgtctgccttggttaggggacatcccgggagcgaaaagcccagcccgtggccgggccggtggaccagcgagcgcgcagcagactgtgcgcggccgcggcgagggcggggaagaaaaccaccctgtttcctctccggcccccaccgcggatcatgtaccaggattatcccgggaactttgacacctcgtcccggggcagcagcggctctcctgcgcacgccgagtcctactccagcggtggcggcggccagcagaagttccgggtagatatgcctggctcgggcagtgccttcatccccacgatcaacgccatcaccaccagccaggacctgcagtggatggtacagcccacagtgatcacctccatgtccaatccctatccacgctcacatccctacagtcccctgccaggcctggcttcagtccctgggcacatggctctccccagacctggagtgatcaagaccatcggtaccaccgtgggccgcagaaggagagatgagcagctgtctcctgaggaggaggagaagcgtcgaatccggagggagagaaacaagctagctgcagccaagtgtcggaaccgtcgccgggagctgacagagaagctgcaggcggagaccgaggagctggaagaggagaagtctgggctgcagaaagagattgctgagctgcagaaggagaaggagaagctagagttcatgttggtggctcacggccccgtgtgcaaaatcagccccgaggaacgccgatcgccccccacctccgggctgcagtccttgcgcggtacgggcagtgccgttggccccgtggtggtgaagcaggagcctcccgaagaggacagcccctcttcctcagcagggatggacaagacccagcgctctgtcatcaagcccatcagcatcgccgggggtggtttctacggggaagagcctctgcacacccccatcgtggtgacctccacgcctgccatcactcccggcacttcaaaccttgtcttcacctaccccaatgtcctggagcaggagtcgccttcgtcgccctcagagtcctgctccaaggctcaccgcagaagcagtagcagtggggaccagtcatcagactccttgaactcccccacacttctagccctgtaaccctgtgctcctctggccagctctggaggaatcctcatccatcattaccacacccttcccagggaccagcacctttaagcactccagggccttgagggaataaggggcccctggccctggggacccacccggtgggacttagtggtgagacactggttgatctcttagaagccctgggacacgccctccctcattcatcttgcaagcgaagcgaatcctttttcttgagaagcctcagagaacttggtttgtggactcagacgcgcctgcggctacccccacctcaccccacccccccctcttctgaagccccgccccctggagctggcccggatggtttctgtccctttgttatccgttgtctctgaagtggacagtatcctttcctgccccgccaagcatgctcagtgccttttggtttcccctccttcccctagattcagttttgctccctctgggtttttataagatttgccatgacatttcatctgggtggtccagatagtaaagcgcttttatttcc

SEQ ID NO: 2 - protein, 326 aa, Mus musculus
MYQDYPGNFDTSSRGSSGSPAHAESYSSGGGGQQKFRVDMPGSGSAFIPTINAITTSQDLQWMVQPTVITSMSNPYPRSHPYSPLPGLASVPGHMALPRPGVIKTIGTTVGRRRRDEQLSPEEEEKRRIRRERNKLAAAKCRNRRRELTEKLQAETEELEEEKSGLQKEIAELQKEKEKLEFMLVAHGPVCKISPEERRSPPTSGLQSLRGTGSAVGPVVVKQEPPEEDSPSSSAGMDKTQRSVIKPISIAGGGFYGEEPLHTPIVVTSTPAITPGTSNLVFTYPNVLEQESPSSPSESCSKAHRRSSSSGDQSSDSLNSPTLLAL

SEQ ID NO: 3 - DNA, 4015 bp, Homo sapiens
cgaacgagcggcgctcggcggggacagaaagagggagagagagagagagagagagggagaggcgcggccgggcgaggcgggcccgtccgggagcgggctccggggaaggggtgcgggtctgggcgccggagcggggagcggggccgcgtccctctcagcgccagctctacttgagccccacgagccgctgtccccctggcgcgctcggggccgcgggacgggcgcacgccgccttctcctagtcaagtatccgagccgccccgaaactcgggcggcgagtcggccacgggaagtttattctccggctccttttctaaaaggaagaaacagaagtttctcccagcggacagcttttctttccgcctttttggccctgtctgaaatcgggggtccccagggctggcaggccaggctcgctgggctcctaatcttttttttaatttccaatttttgattgggccgtgggtccccgctgagctccggctgcgcgcgggggcgggagggcgcgcgcaggggagggaccgagagacgcgccgactttttagagggagggatcgggtggacaactggtcccgcggcgctcgcagagccggaaagaagtgctgtaagggacgctcgggggacgctgttcctgaggtgtcgccgcctccctgtcctcgccctccgcggtgggggagaaacccaggagcgaagcccagagcccgcggcgcggccggcggacgaacgagcgcgcagcagccggtgcgcggccgcggcgagggcgggggaagaaaaacaccctgtttcctctccggcccccaccgcggatcatgtaccaggattatcccgggaactttgacacctcgtcccggggcagcagcggctctcctgcgcacgccgagtcctactccagcggcggcggcggccagcagaaattccgggtagatatgcctggctcaggcagtgcattcatccccaccatcaacgccatcacgaccagccaggacctgcagtggatggtgcagcccacagtgatcacctccatgtccaacccataccctcgctcgcacccctacagccccctgccgggcctggcctctgtccctggacacatggccctcccaagacctggcgtgatcaagaccattggcaccaccgtgggccgcaggaggagagatgagcagctgtctcctgaagaggaggagaagcgtcgcatccggcgggagaggaacaagctggctgcagccaagtgccggaaccgacgccgggagctgacagagaagctgcaggcggagacagaggagctggaggaggagaagtcaggcctgcagaaggagattgctgagctgcagaaggagaaggagaagctggagttcatgttggtggctcacggcccagtgtgcaagattagccccgaggagcgccgatcgcccccagcccctgggctgcagcccatgcgcagtgggggtggctcggtgggcgctgtagtggtgaaacaggagcccctggaagaggacagcccctcgtcctcgtcggcggggctggacaaggcccagcgctctgtcatcaagcccatcagcattgctgggggcttctacggtgaggagcccctgcacacccccatcgtggtgacctccacacctgctgtcactccgggcacctcgaacctcgtcttcacctatcctagcgtcctggagcaggagtcacccgcatctccctccgaatcctgctccaaggctcaccgcagaagcagtagcagcggggaccaatcatcagactccttgaactcccccactctgctggctctgtaacccagtgcacctccctccccagctccggagggggtcctcctcgctcctccttcccagggaccagcaccttcaagcgctccagggccgtgagggcaagagggggacctgccaccagggagcttcctggctctgggggacccaggtgggacttagcagtgagtattggaagacttgggttgatctcttagaagccatgggacctcctccctcattcatcttgcaagcaaatcccatttcttgaaaagccttggagaactcggtttggtagacttggacatctctctggcttctgaagagcctgaagctggcctggaccattcctgtccctttgttaccatactgtctctggagtgatggtgtccttccctgccccaccacgcatgctcagtgccttttggtttcaccttccctcgacttgaccctttcctcccccagcgtcagtttcactccctcttggtttttatcaaatttgccatgacatttcatctgggtggtctgaatattaaagctcttcatttctggagatggggcagcaggtggctcttctgctggggctgacttgtccagaaggggacaaagtgcaatacagagccttccctaccctgacgcctcccagtcatcatctccagaactcccagcggggctccctgagctctcaaggagatgctgccatcactgggaggctcagaggacccttcctgcccaccttcggagacggcttctggaggaacggcttggccagaagacagggtgtgagtgagacagtggggcacaggttgggtttgccaaacgcctaattaccaggccaggaagcatgccaacaaagccacacgggtgtcctagccagcttcccttcacctggtgtcttgagtagggcgtctcctgtaattactgccttgccattctgcccctggacccttctctccggaccagggaggcgtccctccctaggagccacacattatactccaagtccctgccgggctccgcctttcccccaccctggctctcagggtgacgccacccacagagatttaatgagcgtgggcctggaccttccccagatgctgccaggcagcccctccccaagcctcaaagaagcatttgctgaggatggagaggcaggggagggaggcgggaggccgtcactggagtggcgtctgcagcagctgctgccccagcacccgctcagcctgtcctggctgctcacctccccgcagggcaccgggcctttcctgccctctgtggtcatctgccacctgctggatcaagtgctttctcttttacactcccctgtccccaccccagtgcactcttctggcccaggcagcaagcaagctgtgaacagctggcctgagctgtcgctgtggcttgtggctcatgcgccattcctggttgtctgttgaatctttctggctgctggaattggagataggatgttttgcttcccactgcaggagagctgccccctttcacggggttggggaagggtccccctggcctccagcaggagcacagctcagcagggtccctgctgcccacccctctgagccttttctccccagggtatggctcctgctgagtttcttgtccagcagggccttgacaggaatccagggagtagctcctggccagaaccagcctctgcggggcttgtgctctgcaaagactctgctgctggggattcagctctagaggtcacagtatcctcgtttgaaagataattaagatcccccgtggagaaagcagtgacacattcacacagctgttccctcgcatgttatttcatgaacatgacctgttttcgtgcactagacacacagagtggaacagccgtatgcttaaagtacatgggccagtgggactggaagtgacctgtacaagtgatgcagaaaggagggtttcaaagaaaaaggattttgtttaaaatactttaaaaatgttatttcctgcatcccttggctgtgatgcccctctcccgatttcccaggggctctgggagggacccttctaagaagattgggcagttgggtttctggcttgagatgaatccaagcagcagaatgagccaggagtagcaggagatgggcaaagaaaactggggtgcactcagctctcacaggggtaatcatctcaagtggtatttgtagccaagtgggagctattttcttttttgtgcatatagatatttcttaaatgaaaaaaaaaaaaaaaaaaaaaaaaaa

SEQ ID NO: 4 - protein, 326 aa, Homo sapiens
MYQDYPGNFDTSSRGSSGSPAHAESYSSGGGGQQKFRVDMPGSGSAFIPTINAITTSQDLQWMVQPTVITSMSNPYPRSHPYSPLPGLASVPGHMALPRPGVIKTIGTTVGRRRRDEQLSPEEEEKRRIRRERNKLAAAKCRNRRRELTEKLQAETEELEEEKSGLQKEIAELQKEKEKLEFMLVAHGPVCKISPEERRSPPAPGLQPMRSGGGSVGAVVVKQEPLEEDSPSSSSAGLDKAQRSVIKPISIAGGFYGEEPLHTPIVVTSTPAVTPGTSNLVFTYPSVLEQESPASPSESCSKAHRRSSSSGDQSSDSLNSPTLLAL